(19) United States
(12) Patent Application Publication
(10) Pub. No.: US 2022/0084685 A1
(43) Pub. Date: Mar. 17, 2022

(54) MEDICAL LOGISTIC PLANNING SOFTWARE

(71) Applicant: The United States of American as Represented by the Secretary of the Navy, Silver Spring, MD (US)

(72) Inventors: Michael Galameau, San Diego, CA (US); Vern Wing, San Diego, CA (US); Jonny Brock, Brownsboro, AL (US); Edwin D'Souza, Oceanside, CA (US); Trevor Elkins, San Diego, CA (US); Ray Mitchell, huntsville, AL (US); Tracy Negus, San Diego, CA (US); Ralph Nix, Escondido, CA (US); Jay Walker, San Diego, CA (US); James Zouris, San Diego, CA (US); Chirstopher G. Blood, San Diego, CA (US)

(73) Assignee: The United States of American as Represented by the Secretary of the Navy, Silver Spring, MD (US)

(21) Appl. No.: 17/535,613

(22) Filed: Nov. 25, 2021

Related U.S. Application Data
(63) Continuation of application No. 15/004,022, filed on Jan. 22, 2016, now abandoned.
Application No. 15/004,022 is a continuation-in-part of application No. 14/192,521, filed on Feb. 27, 2014, now Pat. No. 10,706,129.
(60) Provisional application No. 61/769,805, filed on Feb. 27, 2013.
(60) Provisional application No. 60/107,077, filed on Nov. 4, 1998.

(51) Int. Cl. G16H 50/50

(52) U.S. Cl. CPC G16H 50/50; G16H 50/30

(57) ABSTRACT

The present invention is a software, methods, and system for creating and editing a medical logistics simulation model and for presenting the simulation model simulated within a military or disaster relief scenario. A user interface that allows a user to enter and edit platforms and associated attributes for a simulation model. The system runs the simulation model based on user input and historical data stored in databases using the inventive software. The present invention provides an output for allowing a user to view casualty rates, patient streams, and medical requirements or any other desired aspect of the simulation model.

### CROSS-REFERENCE TO RELATED APPLICATIONS

This application is a continuation of U.S. patent application Ser. No. 15/004,022, filed Jan. 22, 2016, which is a continuation-in-part application of patent application Ser. No. 14/192,521 filed on Feb. 27, 2014 (U.S. Pat. No. 10,706,129), and claims priority to U.S. Provisional Application No. 62/107,072 filed on Jan. 23, 2015.

### STATEMENT REGARDING FEDERALLY SPONSORED RESEARCH OR DEVELOPMENT

This invention was made with Government support under contracts W911QY-11-D-0058 and N62645-12-C-4076 that were awarded by the OSD DHA, OPNAV (N81), and the Joint Staff. The Government has certain rights in the invention.

### BACKGROUND

In today's military and emergency response operations, medical planners frequently encounter problems in accurately estimating illnesses, casualties and mortalities rates associated with an operation. Largely relying on anecdotal evidences and limited historical information of similar operations, medical planners and medical system analysts don't have a way to scientifically and accurately projecting medical resources, and personnel requirements for an operational scenario. Inadequate medical logistic planning can lead to shortage of medical supplies, which may significantly impact the success of any military, humanitarian or disaster relief operation and could result in more casualties and higher mortality rates. Therefore, there is an urgent need for the development of a science based medical logistics and planning tool.

Before the development of this invention, some useful, but not comprehensive medical modeling and simulation tools were used in attempts to virtually determine the minimum capability necessary in order to maximize medical outcomes, and ensure success of the military medical plan, such as Ground Casualty Projection System (FORECAS) and the Medical Analysis Tool (MAT).

FORECAS produced casualty streams to forecast ground causalities. It provide medical planners with estimates of the average daily casualties, the maximum and minimum daily casualty load, the total number of casualties across an operation, and the overall casualty rate for a specified ground combat scenario. However, FORECAS does not specify the type of injury or take into account the time required for recovery.

MAT and later the Joint Medical Analysis Tool (JMAT) consisted of two modules. One module was designed as a requirements estimator for the joint medical treatment environment while the other module was a course of action assessment tool. Medical planners used MAT to generate medical requirements needed to support patient treatment within a joint warfighting operation. MAT could estimate the number of beds, the number of operating room tables, number and type of personnel, and the amount of blood required for casualty streams, but was mainly focused at the [symbol] eater Hospitalization level of care are definitive cares, which comprises of combat support hospitals in theaters (CSH) but does not include the forward medical facilities like the Battalion Aid Station or Surgical companies. Furthermore, MAT treated the theater medical capabilities as consisting of three levels of care, but failed to take into account medical treatment facilities (MTFs) at each level, their spatial arrangements on a battlefield, nor the transportation assets necessary to interconnect the network. Because MAT was a DOD-owned software program, it also did not include a civilian model. As MAT was designed to be used as a high-level planning tool, it does not have the capability to evaluate forward medical capabilities, or providing a realistic evaluation of mortality. JMAT, the MAT successor, failed Verification and Validation testing in August 2011, and the program were cancelled by the Force Health Protection Integration Council. Other simulations were described by in report by Von Tersch et al. [1].

The existing simulation and modeling software provide useful information for preparing for a military mission. However, they lack the capability to model the flow of casualties within a specific network of treatment facilities from the generation of casualties, and through the treatment networks, and fails to provide critical simulation of the treatment times, and demands on consumable supplies, equipment, personnel, and transportation assets. There are no similar medical logistic tools are on the market for civilian medical rescue and humanitarian operations planning.

Military medical planners, civilian medical system analysts, clinicians and logisticians alike need a science-based, repeatable, and standardized methodology for predicting the likelihood of injuries and illnesses, for creating casualty estimates and the associated patient streams, and for estimating the requirements relative to theater hospitalization to service that patient stream. These capability gaps undermine planning for medical support that is associated with both military and civilian medical operations.

### SUMMARY OF INVENTION

An objective of this invention is the management of combat, humanitarian assistance (HA), disaster relief (DR), shipboard, and fixed base PCOFs (patient condition occurrence frequencies) distribution Tables.

Another objective of this invention is estimation of casualties in HA and DR missions, and in ground, shipboard, and fixed-base combat operations.

Yet another objective of this invention is the generation of realistic patient stream simulations for a HA and DR missions, and in ground, shipboard, and fixed-base combat operations.

Yet another objective of this invention is the estimation of medical requirements and consumables, such as operations rooms, intensive care units, and ward beds, evacuations, critical care air transport teams and blood products, based on anticipated patient load.

### DETAILED DESCRIPTION OF THE DRAWINGS

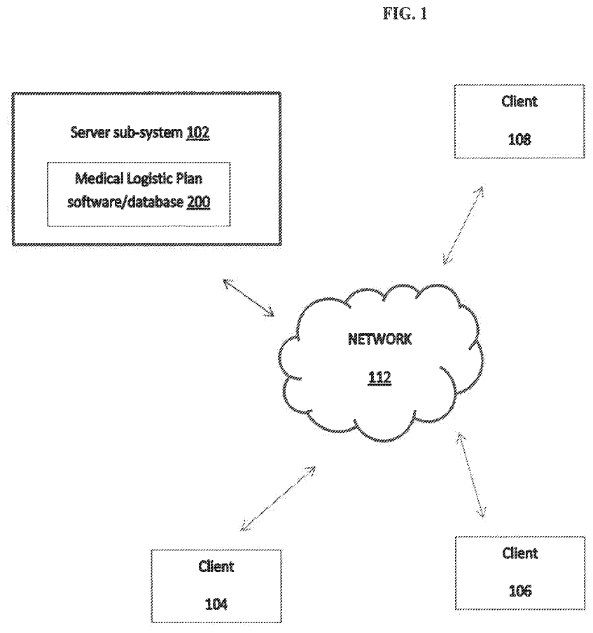

FIG. 1 is a schematic view of a computer system (that is, a system largely made up of computers) in which software and/or methods of the present invention can be used.

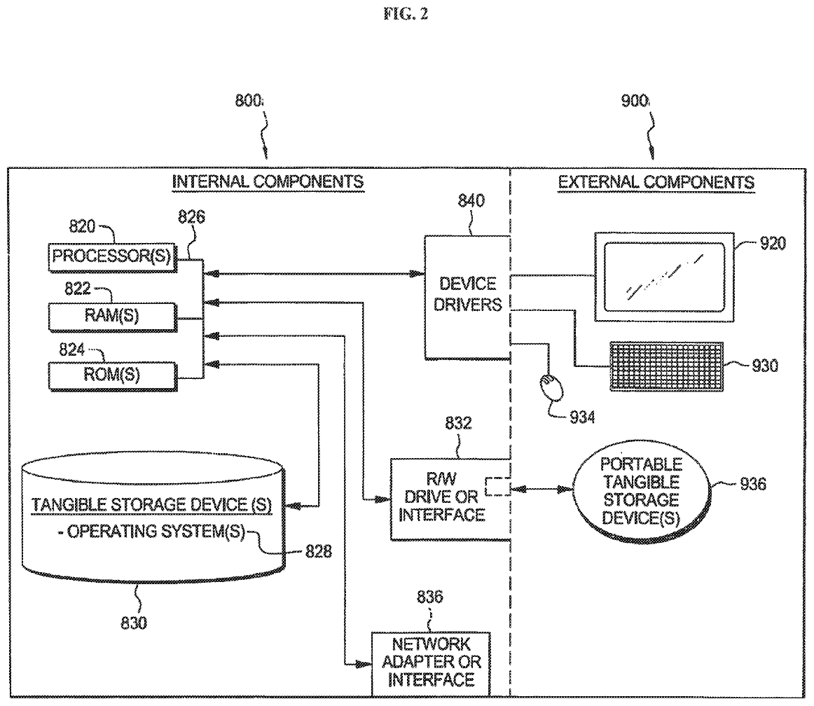

FIG. 2 is a schematic view of a computer sub-system that is a constituent sub-system) of the computer system of FIG. 1), which represents a first embodiment of computer system for medical logistic planning according to the present invention.

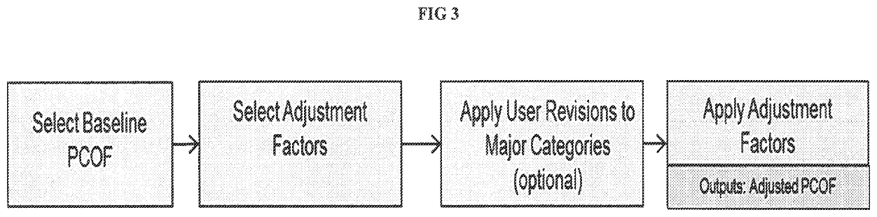

FIG. 3 High-level process diagram for PCOF tool.

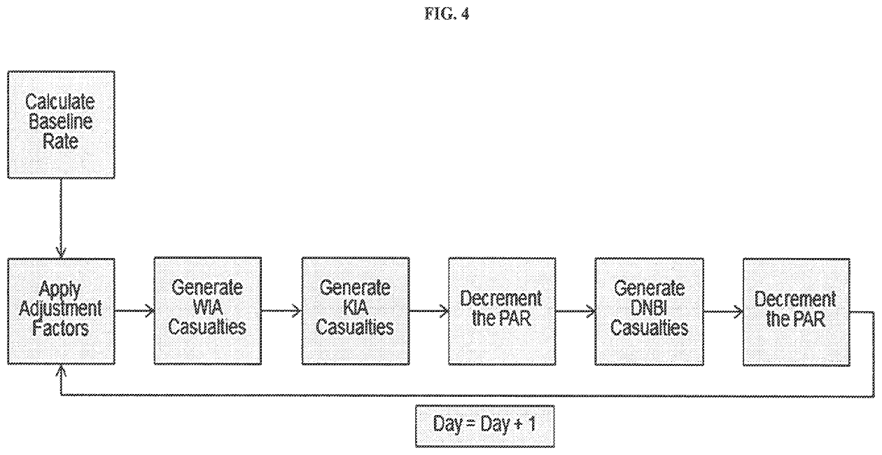

FIG. 4 High-level process diagram for CREsT.

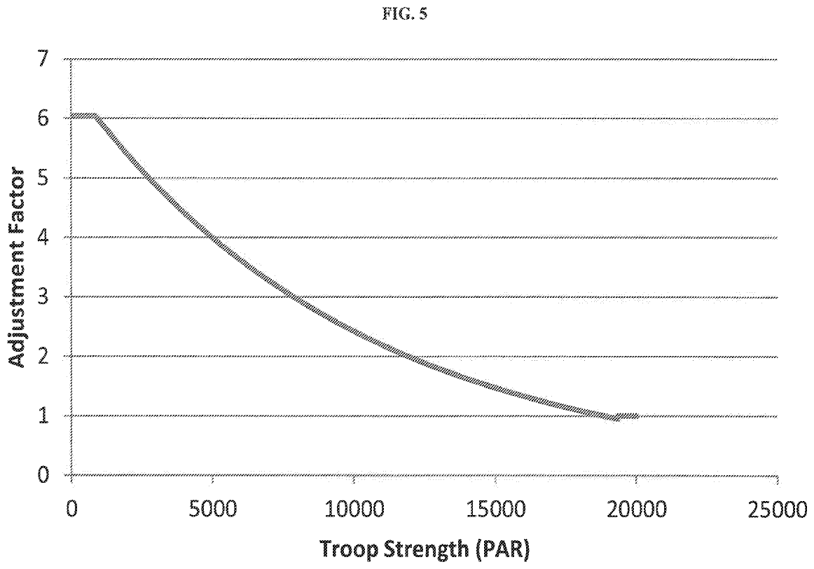

FIG. 5 Diagram showing troop strength adjustment factor.

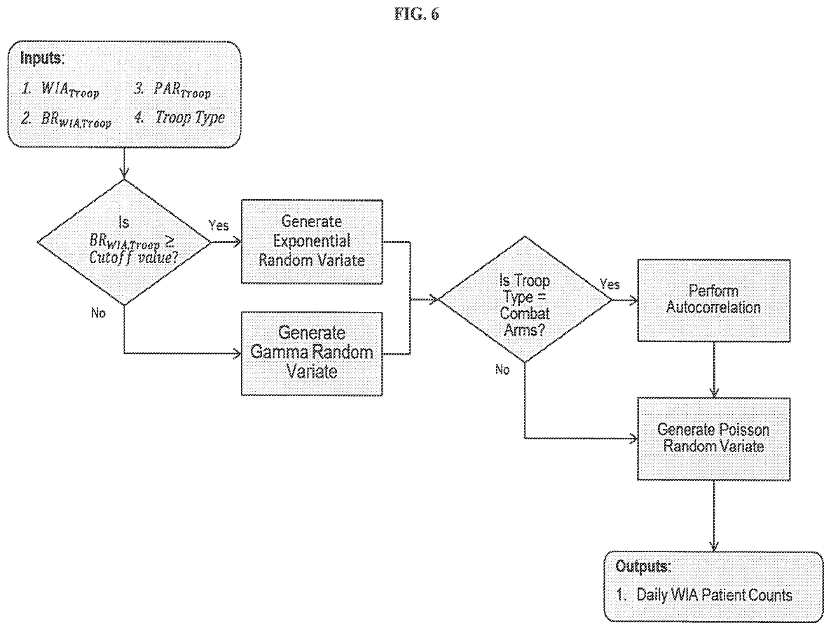

FIG. 6 The logic diagram showing the process of Generation of wounded in action (WIA) casualties (i.e. Daily WIA patient counts).

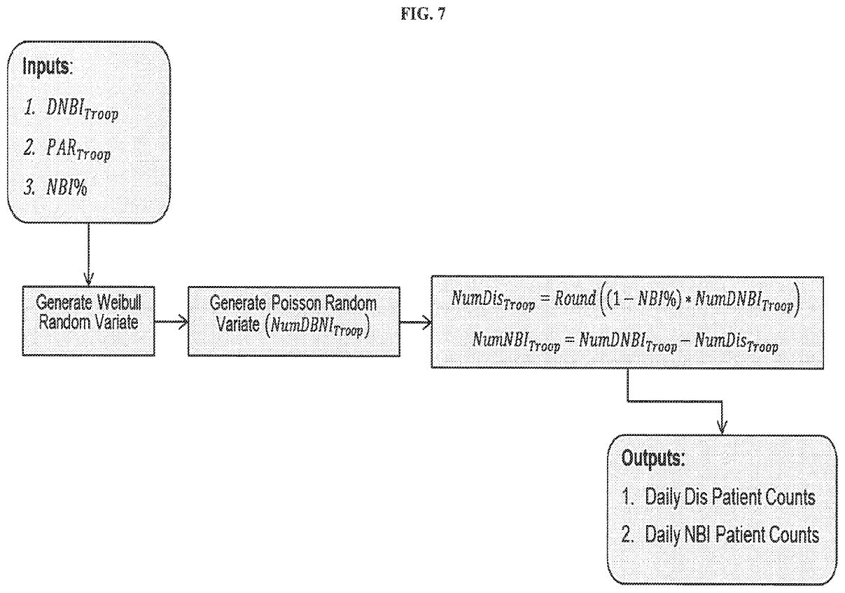

FIG. 7 The logic diagram showing the process of Calculating (disease and nonbattle injuries) DNBI Casualties.

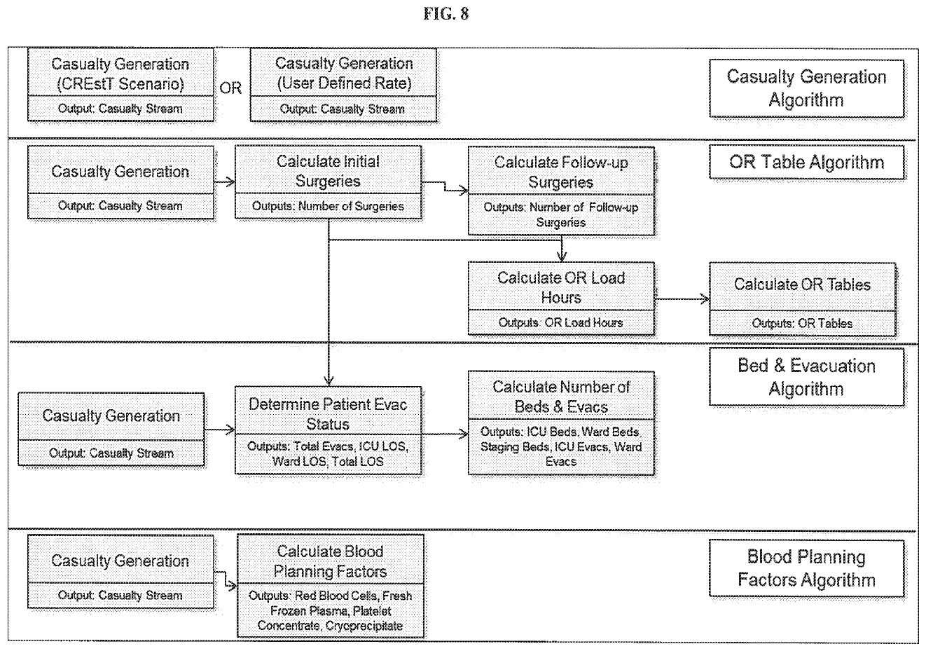

FIG. 8 High-level process diagram for Expeditionary Medicine Requirements Estimator (EMRE).

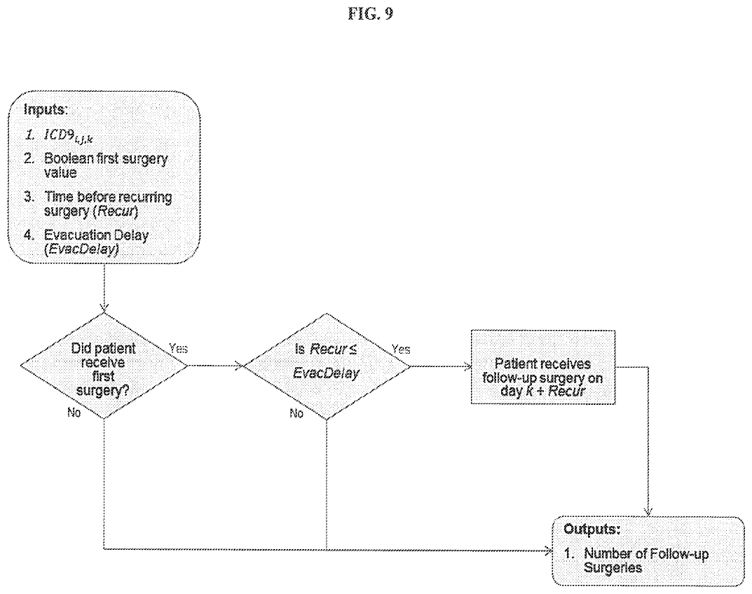

FIG. 9 The logic diagram showing the process of determining casualties requiring follow-up surgery.

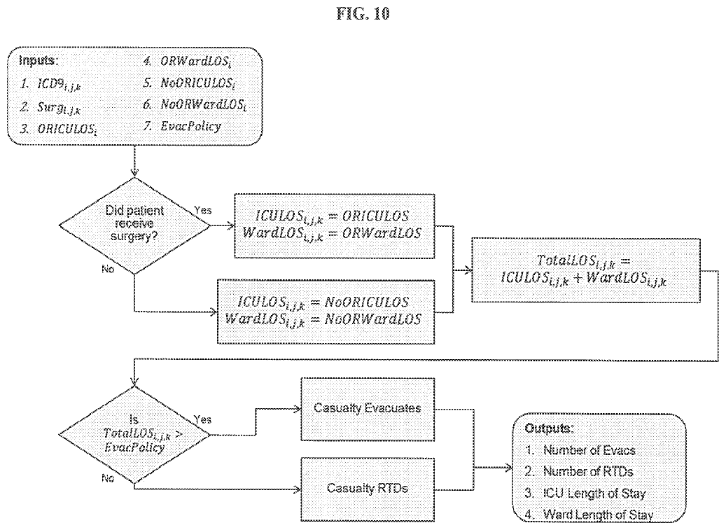

FIG. 10 The logic diagram showing the process of determining casualties requiring for evacuation.

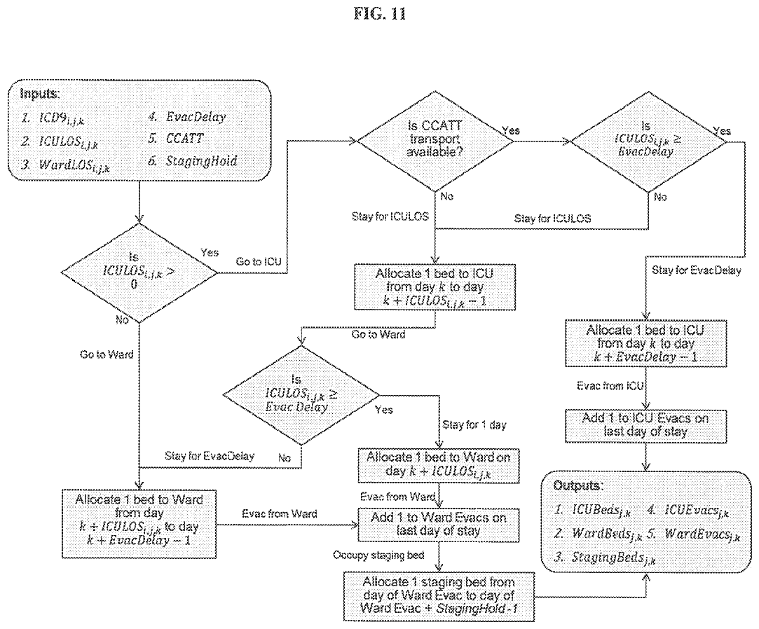

FIG. 11 The logic diagram showing how EMRE calculates evacuation (Evacs) and hospital beds status.

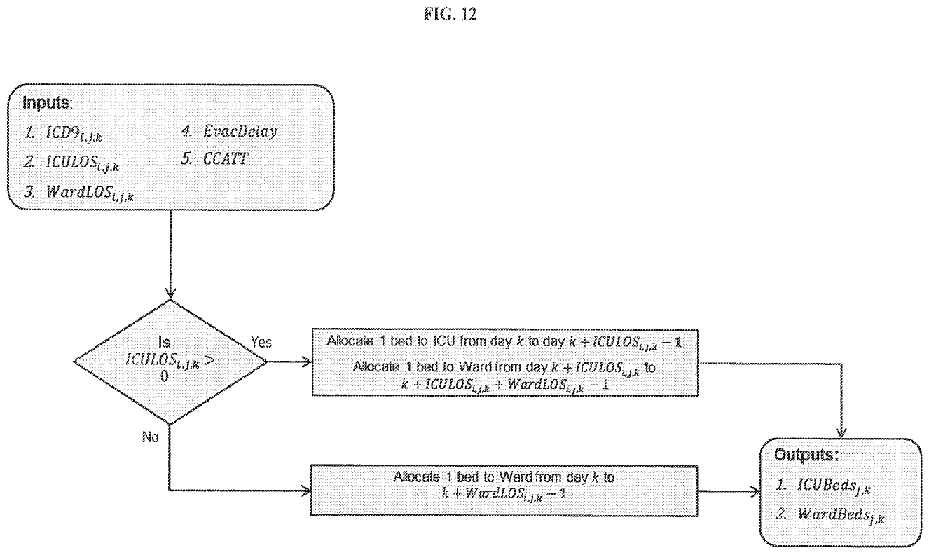

FIG. 12 The logic diagram showing how EMRE determines casualty will return to duty (RTD).

### DETAILED DESCRIPTION OF THE INVENTION

### Definitions

Common data are data stored in one or more database of the invention, which include EMRE common data, CREstT common data, and PCOF common data. The application contains tables labeling inputs used in different software modules and identify them if they are common data.

Patient Conditions (PCs) are used throughout MPTk to identify injuries and illnesses. The PCOF Tool is used to determine the probability of each patient condition occurring. CREstT creates a patient stream by assigning a PC to each casualty it generates. EMRE determines theater hospitalization requirements based on the resources required to treat each PC in a patient stream. All patient conditions in MPTk are codes from the International Classification of Diseases, Ninth Revision (ICD-9). MPTk currently supports 404 ICD-9 codes. 336 of them are codes selected by the Defense Medical Materiel Program Office (DMMPO). An additional 68 codes were added to this set to provide better coverage, primarily of diseases. In each of the three tools, the user can select to use the full set of PC codes or only the 336 DMMPO PC codes.

PCOF scenarios organize patient conditions and their probability of occurrence into major categories and subcategories, and allow for certain adjustment factors to affect the probability distribution of patient conditions. While baseline PCOF scenarios cannot be directly modified by the user, they can be copied and saved with a new name to create derived PCOF scenarios.

Derived PCOF scenarios, created from any baseline PCOF scenario, also organize the probability of patient conditions into major categories and subcategories affected by adjustment factors, all of which may be edited directly by the user.

Unstructured PCOF scenarios provide the user with a list of patient conditions and their probability of occurrence, but do not contain further categorization and are not adjusted by other factors. MPTk includes a number of unstructured PCOF scenarios built and approved by NHRC, and these may not be directly modified by the user. However, the user may copy and save unstructured PCOF scenarios as new unstructured PCOF scenarios, and these may be modified by the user. Users may also create new unstructured PCOF scenarios from scratch.

Any new derived or unstructured PCOF scenarios are saved to the database, and will appear in the PCOF scenario list with the baseline and unstructured PCOF scenarios that shipped with MPTk.

A scenario includes parameters of a planned medical support mission. The scenario may be created in PCOF, CREstT or EMRE modules. A user establishes a scenario by providing inputs and defines parameters of each individual module.

Casualty count is each simulated casualty in MPTk, which may be labeled and maybe assigned a PC code.

[symbol] eater Hospitalization level of care are definitive care, which comprises of combat support hospitals in theaters (CSH) but does not include the forward medical facilities like the Battalion Aid Station or Surgical companies.

This invention relates to a system, method and software for creating military and civilian medical plans, and simulating operational scenarios, projecting medical operation estimations for a given scenario, and evaluating the adequacy of a medical logistic plan for combat, humanitarian assistance (HA) or disaster relief (DR) activities.

I. Computer System and Hardware

FIG. 1 shows an embodiment of the inventive system. A computer system 100 includes a server computer 102 and several client computers 104, 106, 108, which are connected by a communication network 112. Each server computer 102, is loaded with a medical planner's toolkit (MPTk) software and database 200. The MPTk software 200 will be discussed in greater detail, below. While the MPTk software and database of the present invention is illustrated as entailed entirely in the server computer 102 in this embodiment, the MPTk software and database 200 could alternatively be located separately in whole or in part in one or more of the client computers 104, 106, 108 or in a computer readable medium.

As shown in FIG. 2, server computer 102 is a computing/processing device that includes internal components 800 and external components 900. The set of internal components 800 includes one or more processors 820, one or more computer-readable random access memories (RAMs) 822 and one or more computer-readable read-only memories (ROMs 824) on one or more buses 826, one or more operating systems 828 and one or more computer-readable storage devices 830. The one or more operating systems 828 and MPTk software/database 200 (see FIG. 1) are stored on one or more of the respective computer-readable storage devices 830 for execution by one or more of the respective processors 820 via one or more of the respective RAMs 822 (which typically include cache memory). In the illustrated embodiment, each of the computer-readable storage devices 830 is a magnetic disk storage device of an internal hard drive. Alternatively, each of the computer-readable storage devices 830 is a semiconductor storage device such as ROM 824, EPROM, flash memory or any other computer-readable storage device that can store but does not transmit a computer program and digital information.

Set of internal components 800 also includes a (read/write) R/W drive or interface 832 to read from and write to one or more portable computer-readable storage devices 936 that can store, but do not transmit, a computer program, such as a CD-ROM, DVD, memory stick, magnetic tape, magnetic disk, optical disk or semiconductor storage device. MPTk software/database (see FIG. 1) can be stored on one or more of the respective portable computer-readable tangible storage devices 936, read via the respective R/W drive or interface 832 and loaded into the respective hard drive or semiconductor storage device 830. The term “computer-readable storage device” does not include a signal propagation media such as a copper cable, optical fiber or wireless transmission media.

Set of internal components 800 also includes a network adapter or interface 836 such as a TCP/IP adapter card or wireless communication adapter (such as a 4G wireless communication adapter using OFDMA technology). MPTk (see FIG. 1) can be downloaded to the respective computing/processing devices from an external computer or external storage device via a network (for example, the Internet, a local area network or other, wide area network or wireless network) and network adapter or interface 836. From the network adapter or interface 836, the MPTk software and database in whole or partially are loaded into the respective hard drive or semiconductor storage device 830. The network may comprise copper wires, optical fibers, wireless transmission, routers, firewalls, switches, gateway computers and/or edge servers.

Set of external components 900 includes a display screen 920, a keyboard or keypad 930, and a computer mouse or touchpad 934. Sets of internal components 800 also includes device drivers 840 to interface to display screen 920 for imaging, to keyboard or keypad 930, to computer mouse or touchpad 934, and/or to display screen for pressure sensing of alphanumeric character entry and user selections. Device drivers 840, R/W drive or interface 832 and network adapter or interface 836 comprise hardware and software (stored in storage device 830 and/or ROM 824).

The invention also include an non-transitory computer-readable storage medium having stored thereon a program that when executed causes a computer to implement a plurality of modules for generate estimates of casualty, mortality and medical requirements of a future medical mission based at least partially on historical data stored on the at least one database, the plurality of modules comprising:
A) a patient condition occurrence frequency (PCOF) module that
   i) receives information regarding a plurality of missions of a predefined scenario including PCOF data represented as a plurality sets of baseline PCOF distributions for the plurality of missions;
   ii) selects a set of baseline PCOF distributions for a future medical mission based on a user defined PCOF scenario;
   iii) determines and presents to the user adjustment factors applicable to the user defined PCOF scenario;
   iv) modifies said selected set of baseline PCOF distributions manually or using one or more PCOF adjustment factors defined by the user to create a set of customized PCOF distributions for the user defined PCOF scenario; and
   v) provides the set of customized PCOF distributions and the corresponding the user defined PCOF scenario and PCOF adjustment factors for storage and presentation;

Various executable programs (such as PCOF, CREsT, and EMRE Modules of MPTk, see FIG. 1) can be written in various programming languages (such as Java, C+) including low-level, high-level, object-oriented or non object-oriented languages. Alternatively, the functions of the MPTk can be implemented in whole or in part by computer circuits and other hardware (not shown).

The database 200 comprises PCOF common data, CREstT common data and EMRE common data. The common data are developed based on historical empirical data, and subject matter expert opinions. For example, empirical data were used to develop an updated list of patient conditions for use in modeling and simulation, logistics estimation, and planning analyses. Multiple Injury Wound codes were added to improve both scope and coverage of medical conditions. Inputs were identified as Common Data in tables throughout this application to distinguish from inputs there were user defined or inputted.

For many years, analysts have used a standardized list of patient conditions for medical modeling and simulation. This list was developed by the Defense Health Agency Medical Logistics (DHA MEDLOG) Division, formerly known as the Defense Medical Standardization Board, for medical modeling and simulation. This subset of International Classification of Diseases, 9th Revision (ICD-9) diagnostic codes was compiled before the advent of modern health encounter databases, and was intended to provide a comprehensive description of the illnesses and injuries likely to afflict U.S. service personnel. Medical encounters from recent contingency operations, were compared to the Clinical Classification Software (CCS; 2014), a diagnosis and procedure categorization scheme developed by the Agency for Healthcare Research and Quality, to establish the hybrid database as an authoritative reference source of healthcare encounters in the expeditionary setting.

II. Computer Programs Modules of the Medical Planner's Toolkit (MPTK)

The inventive MPTk software comprises three modeling and simulation tools: the Patient Condition Occurrence Frequency Tool (PCOF), the Casualty Rate Estimation Tool (CREstT) and the Expeditionary Medicine Requirements Estimator (EMRE). Used independently, the three simulation tools provide individual reports on causality generation, patient stream, and medical planning requirements, which can each be used by medical system analysts or logisticians and clinicians in different phases of medical operation planning. The three stimulation tools can also be used collectively as a toolkit to generate detailed simulations of different medical logistic plan designed for an operational scenario, which can be compared to enhance a medical planner's overall efficiency and accuracy.

A. Patient Condition Occurrence Frequency Tool (PCOF)

The PCOF tool provides medical planners and logisticians with estimates of the distributions of injury and illness types for a range of [symbol] itary operations (ROMO). These missions include combat, noncombat, humanitarian assistance (HA), and disaster relief (DR) operations. Using the PCOF tool, baseline distributions of a patient stream composition may be modified by the user either manually and/or via adjustment factors such as age, gender, country, region to better resemble the patient conditions of a planned operation. A PCOF table can provide the probability of injury and illness at the diagnostic code level. Specifically, each PCOF is a discrete probability distribution that provides the probability of a particular illness or injury. The PCOF tool was developed to produce precise expected patient condition probability distributions across the entire range of military operations. These missions include ground, shipboard, fixed-base combat, and HA and DR non-combat scenarios. The PCOF distributions are organized in three levels: International Classification of Diseases, Ninth Revision (ICD-9) category, ICD-9 subcategory, and patient condition (ICD-9 codes). Example of ICD-9 category, ICD-9 subcategory and patient condition may be dislocation, dislocation of the finger, dislocation of Open dislocation of metacarpophalangeal (joint), respectively. These PCOF distribution tables for combat missions were developed using historical combat data. The major categories and sub-categories for the HA and DR missions were developed using a 2005 datasheet by the International Medical Corps from ReliefWeb (a United Nations Web site). Because the ICD-9 codes from this datasheet is restrictive to that particular mission, the categories, sub-categories, and ICD-9 codes for trauma and disease groups of HA and DR operations are further expanded to account for historical data gathered from [symbol] er sources, and modified to be consistent with current U.S. Department of Defense (DoD) medical planning policies. Because the ICD-9 codes are not exclusively used for military combat operations, all DoD military combat ICD-9 codes are used for HA and DR operation planning in conjunction with the additional HA and DR ICD-9 codes in the present invention. The PCOF tool can generate a report that may be used to for support supply block optimization, combat scenario medical supportability analysis, capability requirements analysis, and other similar analysis.

The high level process diagram of PCOF is shown in FIG. 3. The PCOF tool includes a baseline set of predefined injury and illness distributions (PCOFs) for a variety of missions. These baseline PCOFs are derived from historical data collected from military databases and other published literature. PCOF tool also allows the import of user-defined PCOF tables or adjustment using user applied adjustment factor.

Each baseline PCOF table specifies the percentage of a patient type in the baseline. In one embodiment of the PCOF tool, there are five patient-type categories: wounded in action (WIA), non-battle injury (NBI), disease (DIS), trauma (TRA), and killed in action (KIA). The user can alter these percentages to reflect the anticipated ratios of a patient steam in a planned operation scenario. Adjustment factors applied at the patient-type level affect the percentage of the probability mass in each patient-type category, but do not affect the distribution of probability mass at the ICD-9 category, ICD-9 subcategory or patient condition levels within the patient-type category. Changes at patient-type level may be entered by the user directly. Patient Type is a member of the set {DIS, WIA, NBI, [symbol] A} and PCT_{DIS}, PCT_{WIA}, PCT_{NBI} and PCT_{TRA} are the proportions of DIS, WIA, NBI, and TRA patients respectively.

Then for ground combat scenarios:

$$P C {T}_{D I S} + P C {T}_{W I A} + P C {T}_{N B I} = 100 \%$$

and for non-combat scenarios:

$$P C {T}_{D I S} + P C {T}_{T R A} = 100 \%$$

The PCOF tool also allows users to make this type of manual adjustment at the ICD-9 category and ICD-9 subcategory levels. At each level, total probability of each level (patient-type, ICD-9 category or ICDR-9 subcategory) must add up to 100% whether the adjustment is accomplished manually or through adjustment factors. In an embodiment, adjustment factors are applied at the ICD-9 category (designated as Cat in all equations). The equation below shows the manner in which adjustment factors (AFs) are applied.

```
  Adjusted_ICD9_Cat_{i,j} = Baseline_ICD9_Cat, * AF_{i,j}
Where:
  i is the index of ICD-9 categories,
  j is the index of adjustment factors,
  where j ϵ {age, gender, region, season, climate, income},
  Adjusted ICD9_Cat_{i,j} is the adjusted probability mass in ICD-9 category i due to
  adjustment factor AF_{i,j},
  Baseline ICD9_Cat, is the baseline probability mass in ICD-9 category i, and
  AF_{i,j} is the adjustment factor for an ICD-9 category due to adjustment factor j.

[symbol]

```

The change in each ICD-9 category is calculated for each adjustment factor that applies to that category. The manner in which this calculation is performed depends on the specific application of the adjustment factor. While some adjustment factors adjust all ICD-9 categories directly, a select few adjustment factors adjust certain ICD-9 categories, hold those values constant, and normalizes the remainder of the distribution. For the adjustment factors who adjust categories directly, the change calculation is performed according to the following:

$$Change\_ICD9 {\_Cat}_{i , j} = Adjusted\_ICD9 {\_Cat}_{i , j} - Baseline\_ICD9 {\_Cat}_{i} ,$$

For the adjustment factors which hold certain values constant, the calculation is performed in the following manner.

$$Change\_ICD9 {\_Cat}_{i , j} = Norm ( Adjusted\_ICD9 {\_Cat}_{i , j} ) - Baseline\_ICD9 {\_Cat}_{i} ,$$

where Change_ICD9_Cat_{i,j} is the change in the baseline value for ICD-9 category i due to adjustment factor. Norm( ) refers to the normalization procedure expressed in detail in the section describing the adjustment factor for response phase.
The total adjustment to ICD-9 category i is:

$${Total\_adj}_{i} = {\sum }_{j} Change\_ICD9 {\_Cat}_{I , j}$$

Once all adjustment factors have been applied and their corresponding total adjustments (Total_adjᵢ) calculated, they are applied to the baseline values (Baseline_ICD9_Catᵢ) to arrive at the raw adjusted value. This value is calculated as follows:

$$Raw\_Adj \_Val \_ICD9 {\_Cat}_{i} = {Total\_adj}_{i} + Baseline\_ICD9 {\_Cat}_{i} , ∀ i$$

The ICD-9 categories are renormalized as follows:

$$Final\_ICD9 {\_Cat}_{i} = Raw\_Adj \_Val \_ICD9 {\_Cat}_{i} \text{/} {\sum }_{i} Raw\_Adj \_Val \_ICD9 {\_Cat}_{i} , ∀ i$$

The adjusted patient condition probability (Pc adjusted) is calculated as follows:

$$Pc\_adjusted = Pc\_baseline * ICD9\_sub \_category * Final\_ICD9 {\_Cat}_{i}$$

Where:
   Pc_baseline is the value of the proportion of the PC among the other PC's in ICD-9 subcategory i.
   ICD9_sub_category is the value of the proportion of the ICD-9 subcategory among the subcategories that make up ICD-9 category i, and
   Final_ICD9_Catᵢ is calculated as above.

Users are able to alter scenario variables from the the graphic user interface ( [symbol] I). The tool calculates the appropriate adjustment factors based on this user input. Not all adjustment factors affect all ICD-9 categories. Furthermore, adjustment factors may not affect all of the injury types within an ICD-9 category. Table 0 displays the adjustment factors that affect patient types by scenario type.

TABLE 1
PCOF Adjustment Factors

| Adjustment | HA | | DR | | Ground Combat | | |
|---|---|---|---|---|---|---|---|
| factors | Disease | Trauma | Disease | Trauma | Disease | NBI | WIA |
| Age | x | x | x | x | | | |
| Gender | x | x | x | x | x | x | x |
| Region | | | | | x | | |
| Response | | | x | x | | | |
| phase | | | | | | | |
| Season | x | | x | | x | | |
| Country | x | x | x | x | | | |

Calculation for each adjustment factors are described in the following sections.

### Adjustment Factor for Age

PCOF types affected: HA, DR
Patient types affected: disease, trauma

The age adjustment factor was determined using the Standard Ambulatory Data Record (SADR); a repository of administrative data associated with outpatient visits by military health system beneficiaries. This data is the baseline population in all calculations below. The data were organized by age into four groups:

1) ages less than 5 years, i=1;

2) ages 5 to 15 years, i=2;

3) ages 16 to 65 years, i=3; and

4) ages greater than 65 years, i=4.

The age adjustment factor is determined as follows:
Let i denote the age group, where i∈{1, 2, 3, 4}
Let m denote the index for ICD-9 categories, where m∈{1, 2, . . . , M} and there are M distinct ICD-9 categories.
Let BaselineAgeᵢ be the percentage of age group i in the population of the baseline distribution.
Let AdjustedAgeᵢ be the user-adjusted percentage of the population in age group i.
Let ICD9_Cat_Age_{i,m} be the percentage of the SADR population in age group i within ICD-9 category m.
The adjustment factors for age are calculated as follows:

$${AF\_Age}_{m} = \frac{{\sum }_{i = 1}^{4} ( Adj u s t e d A g {e}_{i} * ICD9\_Cat {\_Age}_{i , m} )}{{\sum }_{i = 1}^{4} ( Bas e l i n e A g {e}_{i} * ICD9\_Cat {\_Age}_{i , m} )}$$

### Adjustment Factor for Gender

PCOF types affected: HA, DR, and ground combat
Patient types affected: WIA, NBI, disease, and trauma

The gender adjustment factor was derived in a manner similar to the age adjustment factor. The data source for the gender adjustment factor was SADR. The data were organized by gender:

Male, i=0

Female, i=1

The gender adjustment factor is calculated as follows:
Let BaselineGenderᵢ be the percentage of the gender group i in the baseline population, i∈{0,1}.
Let AdjustedGenderᵢ be the user adjusted percentage of the population in gender group i.
Let ICD9_Cat_Gender_{i,m} be the percentage of the SADR population in gender group i within ICD-9 category m.
The adjustment factor is calculated as follows:

$${AF\_Gender}_{m} = \frac{{\sum }_{i = 0}^{1} ( Adj u s t e d G e n d e {r}_{i} * ICD9\_Cat {\_Gender}_{i , m} )}{{\sum }_{i = 0}^{1} ( Bas e l i n e G e n d e {r}_{i} * ICD9\_Cat {\_Gender}_{i , m} )}$$

### OB/GYN Correction

The “OB/GYN Disorders” major category is adjusted in the same manner as all other major categories. However, in the special case where the population is 100% male, the percentage of OB/GYN disorders is automatically set to zero, and all other major categories are renormalized (Recalculated so the percentages add to 100%.

### Adjustment Factor for Region

PCOF types affected: ground combat
Patient types affected: disease

The regional adjustment factor was developed via an analysis of data from World War II. The World War II data was categorized by combatant command (CCMD) and organized into the major disease categories found in the PCOF. The World War II data comprise the baseline population referenced below.

Let CCMD_{Baseline,m} be the percentage of the World War II population comprising ICD-9 category m for the baseline CCMD of the scenario.

Let CCMD_{Adjusted,m} be the percentage of the World War II population comprising ICD-9 category m for the user-adjusted CCMD of the scenario.
The adjustment factor is calculated as follows:

$${AF\_Region}_{m} = \frac{( C C M {D}_{A d j u s t e d , m} )}{( C C M {D}_{B a s e l i n e , m} )}$$

Where AFₘ is the adjustment factor used to transition an ICD-9 category m from CCMD_{Baseline} to CCMD_{Adjusted}.

### Adjustment Factor for Response Phase

PCOF types affected: DR
Patient types affected: disease and trauma

Response phase denotes the time frame within the event when aid arrives. For the purposes of this adjustment factor, response phases were broken down into three time windows and are described below.

1) Early Phase is from the day the event occurs to the following day.

2) Middle Phase is the third day to the 15th day.

3) Late Phase is any time period after the 15th day.

These phases are described in the Pan American Health Organization's manual on the use of Foreign Field Hospitals (2003). Response phase adjustment factors perform two functions. First, they adjust the ratio of disease to trauma. Second, unlike the adjustment factors discussed above, they only adjust the percentages of a small subset of the major categories rather than the entire PCOF. Subject matter expert (SME) input and reference articles were used to develop adjustment factors that adjust the most likely conditions affected by the response phase for both disease and trauma casualties. The conditions are shown in Table 0 and Table 0.

TABLE 2
Disease Major Categories Affected by Response Phase
Disease major category

Gastrointestinal disorders, k = 1
Infectious diseases, k = 2
Respiratory disorders, k = 3
Skin disorders, k = 4

TABLE 3
Trauma Major Categories Affected by Response Phase
Trauma major categories

Fractures, l = 1
Open wounds, l = 2

For the major categories, which are adjusted and held constant, the calculations are as follows.

Let k denote the index for ICD-9 categories adjusted by response phase for disease, where k∈{1, 2, 3, 4} and l denote the same for trauma, where l∈{1, 2}.
Let xₖ be the percentage of major category k, which will be adjusted and held constant.
Let yₙ be the percentage of major category n, which will be normalized such that the distribution sums to 1, where n∈{1, 2, . . . , N}.
Let aₖ be the adjustment factor for major category k for disease and let aₗ be the adjustment factor for major category l for trauma. The calculations for the major categories, which are adjusted and held constant, are calculated according to the formulas below (the example is for disease; the same formulation applies to trauma).

$$\{ \begin{matrix}{x}_{k} {a}_{k} & if { \sum }_{k = 1}^{4} ( {x}_{k} {a}_{k} ) \leq 1 \; 00 \% \\ \frac{{x}_{k} {a}_{k}}{{\sum }_{k = 1}^{4} ( {x}_{k} {a}_{k} )} & if {\sum }_{k = 1}^{4} ( {x}_{k} {a}_{k} ) > 1 \; 00 \%\end{matrix}$$

The calculations for the major categories, which are normalized so that the distribution sums to 1, are as follows (the example is for disease; the same formulation applies to trauma).

$$\{ \begin{matrix}\frac{{y}_{n}}{{\sum }_{n = 1}^{N} ( {y}_{n} )} * ( 1 - {\sum }_{k = 1}^{4} ( {x}_{k} {a}_{k} ) ) & if {\sum }_{k = 1}^{4} ( {x}_{k} {a}_{k} ) < 1 \; 00 \% \\ 0 & if {\sum }_{k = 1}^{4} ( {x}_{k} {a}_{i k} ) \geq 1 \; 00 \%\end{matrix}$$

The adjustment factor was developed via SME input and has no closed form. There are unique adjustment factors for each of the six distinctive combinations of baseline and adjusted response phases.

There is also an adjustment to the disease-to-trauma ratio due to a change in response phase. For any change in response phase, the adjustment factor for disease is inversely proportional to the adjustment factor for trauma. Therefore, if the adjustment factor for disease is 8, the adjustment factor for trauma will be

$$\frac{1}{8} = 0.125 .$$

Table 0 denotes the adjustments to relative disease and trauma percentages. These values are then normalized so that they sum to 100%.

TABLE 4
Response Phase Disease-to-Trauma Ratio Adjustment Factor

| Baseline | Adjusted | Disease | Trauma |
|---|---|---|---|
| response phase | response phase | adjustment factor | adjustment factor |
| Early | Middle | 4 | 0.25 |
| Early | Late | 8 | 0.125 |
| Middle | Early | 0.25 | 4 |
| Middle | Late | 4 | 0.25 |
| Late | Early | 0.125 | 8 |
| Late | Middle | 0.25 | 4 |

### Adjustment Factor for Season

### Top Category Adjustment

PCOF types affected: HA, DR, and ground combat
Patient types affected: disease

The development of the seasonal adjustment factor was performed via the analysis of SADR data for HA and DR scenarios, and from Operation Iraqi Freedom (OIF) and Operation Enduring Freedom (OEF) for ground combat scenarios that had been parsed by season. For ground combat PCOFs, the default season is always “All,” implying that the operation spanned multiple or all seasons. For HA and DR PCOFs, the default season is set respective to the season in which the operation took place. For each combination of seasons in HA and DR scenarios, an odds ratio was developed that measures the likelihood of a condition occurring in the user-adjusted season to a reference season (the baseline).

The HA and DR season adjustment factors is calculated as follows: Let Season_{Baseline,k} be the percentage of the SADR population comprising ICD-9 category k for the scenario's baseline season. Where k denotes the ICD-9 categories from Table 2 Let Season_{Adjusted,k} be the percentage of the SADR population comprising ICD-9 category k for the scenario's user-adjusted season.

### Then:

$${Odds\_Ratio}_{B a s e l i n e , k \to A djusted , k} = \frac{S e a s o {n}_{A djusted , k} * ( 1 - S e a s o {n}_{B aseline , k} )}{S e a s o {n}_{B a s e l i n e , k} * ( 1 - S e a s o {n}_{A d j u s t e d , k} )}$$

and,
AF_HADRSeasonₖ=Odds_Ratio_{Baseline,k→Adjusted,k}

The ground combat season adjustment factor is calculated as follows: Let Season_{Baseline,m} be the percentage of the OIF or OEF population comprising ICD-9 category m for the scenario's baseline season.

Let Season_{Adjusted,m} be the percentage of the OIF or OEF population comprising ICD-9 category m for the scenario's user-adjusted season.

$${AF\_CombatSeason}_{m} = \frac{( S e a s o {n}_{A d j u s t e d , m} )}{( S e a s o {n}_{Baseline , m} )}$$

The ground combat seasonal adjustment factor aligns all of the disease major categories. After adjustment, the major categories are normalized so that the distribution sums to 100%. The HA and DR seasonal adjustment factor, as in the case of the response phase adjustment factor, only affects a specified set of major categories. Specifically, the adjustment factor for season only affects the disease major categories outlined in Table 0. Additionally, as with the response phase adjustment factor, these major categories are adjusted and kept constant while the remainder of the PCOF is normalized.

### Subcategory Adjustment

PCOF types affected: HA, DR, and ground combat
Patient types affected: NBI, TRA

Season is the only adjustment factor which affects PCOFs on the ICD-9 subcategory level. For NBI and TRA patient types, the season adjustment factor changes the relative percentage of the “Heat” and “Cold” subcategories within the “Heat and Cold” top category. Heat injuries are more common during the summer and cold injuries are more common during the winter. As shown in Table 0, the heat and cold subcategory percentages are determined using only the season. Individual PCOFs cannot have heat and cold percentages other than what is shown in the table 5.

TABLE 5
Season Subcategory Adjustments

| Season | Subcategory | Percentage |
|---|---|---|
| All | Heat | 50% |
| All | Cold | 50% |
| Winter | Heat | 5% |
| Winter | Cold | 95% |
| Spring | Heat | 50% |
| Spring | Cold | 50% |
| Summer | Heat | 95% |
| Summer | Cold | 5% |
| Fall | Heat | 50% |
| Fall | Cold | 50% |

### Adjustment Factor for Country

PCOF types affected: HA and DR
Patient types affected: disease and trauma (trauma is adjusted through age and gender only)

The selection of a country in the PCOF tool triggers four adjustment factors. The first adjustment factor combines region and climate. Each country is classified by region according to the CCMD in which it resides. Along with this is a categorizing of climate type according to the Koppen climate classification. Each combination of CCMD and climate was analyzed according to disability adjusted life years (DALYs), which are the number of years lost due to poor health, disability, or early death, and a disease distribution was formed. Each country within the same CCMD and climate combination shares the same DALY disease distribution for this adjustment factor.

The region and climate type adjustment factor is calculated as follows: Let Region_Climate_{Baseline,m} be the percentage of the DALY population comprising ICD-9 category m for the region and climate combination of the baseline country in the selected season. Let Region_Climate_{Adjusted,m} be the percentage of the DALY population comprising ICD-9 category m for the region and climate combination of the user-adjusted country in the selected scenario.

$$AF\_Region {\_Climate}_{m} = \frac{{Region\_Climate}_{A d j u s t e d , m}}{{Region\_Climate}_{Baseline , m}}$$

TABLE 6
Climate Classifications for Country Adjustment Factor
Climate classification

Tropical
Dry/Desert
Temperate
Continental

The second adjustment factor accounts for the impact of economy in the selected country. Each country's economy was categorized according to the human development index. SME input was used to develop adjustment factors for three major categories (Table 0). As in the case of the response phase adjustment factor and HA and DR seasonal adjustment factor, these three major categories are adjusted and held constant while the remainder of the PCOF is renormalized.

TABLE 7
Income Classifications for Country Adjustment Factor
Income classification

Low
Lower Middle
Upper Middle
High

TABLE 8
Disease Major Categories Affected by Income
Disease major categories

Gastrointestinal disorders
Infectious diseases
Respiratory disorders

There is also an adjustment to the disease-to-trauma ratio due to a change in income. The disease and trauma percentages will be adjusted when the selection of a new country changes the income group. 0 denotes the adjustments that will be applied to the disease patient type percentage. After the disease percentage is multiplied by the adjustment factor, the disease and trauma percentages are renormalized to sum to 100%.

TABLE 9
Income Disease-to-Trauma Ratio Adjustment Factor

| | | Disease |
|---|---|---|
| Baseline Income | Current Income | adjustment factor |
| Low | Lower Middle | 1.050 |
| Low | Upper Middle | 1.100 |
| Low | High | 1.150 |
| Lower Middle | Low | 0.952 |
| Lower Middle | Upper Middle | 1.050 |
| Lower Middle | High | 1.100 |
| Upper Middle | Low | 0.909 |
| Upper Middle | Lower Middle | 0.952 |
| Upper Middle | High | 1.050 |
| High | Low | 0.870 |
| High | Lower Middle | 0.909 |
| High | Upper Middle | 0.952 |

Finally, adjustment factors are applied for the change in age and gender. These adjustments are performed in the same manner as user-input changes to age and gender distribution (described above). However, instead of a user-input age or gender distribution, the age and gender distribution of the user-chosen country is used.

B. Casualty Rate Estimation Tool (CREstT)

The Casualty Rate Estimation Tool (CREstT) provides user estimate casualties and injuries resulting from a combat and non-combat event. CREstT may be used to generate casualties estimates for ground combat operations, attacks on ships, attacks on fixed facilities, and casualties resulting from natural disasters. These estimates allow medical planners to assess their operation plans, tailor operational estimates using adjustment factors, and develop robust patient streams best mimicking that expected in the anticipated operation. CREstT also has an interface with the PCOF tool, and can use the distributions stored or developed in that application to produce patient streams. Its stochastic implementation provides users with percentile as well as median results to enable risk assessment. Reports from CREsT may be programed to present data in both tabular and graphical formats. Output data is available in a format that is compatible with EMRE, JMPT, and other tools. The high level process diagram of PCOF is shown in FIG. 4.

### Estimate for Ground Combat Operations

Baseline ground combat casualty rate estimates are based on empirical data spanning from World War II through OEF. Baseline casualty rates are modified through the application of adjustment factors. Applications of the adjustment factors provide greater accuracy in the casualty rate estimates. The CREsT adjustment factors are based largely on research by Trevor N. Dupuy and the Dupuy Institute (Dupuy, 1990). The Dupuy factors are weather, terrain, posture, troop size, opposition, surprise, sophistication, and pattern of operations. The factors included in CREstT are region, terrain, climate, battle intensity, troop type, and population at risk (PAR). Battle intensity is used in CREstT instead of opposition, surprise, and sophistication factors to model enemy strength factors.

Casualty estimates for ground combat operations in CREstT are calculated using the process depicted in FIG. 4. The following sections outline the sub-processes and provide descriptions of inputs and outputs and the algorithms used in the estimation.

### Calculate Baseline Rates

The CREstT baseline rates are the starting point for the casualty generation process. There is a WIA baseline rate which is dependent on troop type, battle intensity, and service and a DNBI baseline rate which is dependent only on troop type.

TABLE 10
Calculate Baseline Rate Inputs

| Variable | | | | |
|---|---|---|---|---|
| Name | Description | Source | Min | Max |
| Troop Type | The generic type of simulated unit. | User- | N/A | N/A |
| | Troop Type ε {Combat Arms, | input | | |
| | Combat Support, Service Support}. | | | |
| Battle | The level of intensity at which the | User- | N/A | N/A |
| Intensity | battle will be fought. Battle | input | | |
| | Intensity ε {None, Peace Ops, | | | |
| | Light, Moderate, Heavy, Intense, | | | |
| | User Defined}. | | | |
| Service | The military service associated | User- | N/A | N/A |
| | with the scenario. Service ε | input | | |
| | {Marines, Army}. | | | |
| User | An optional user defined WIA rate | User- | 0 | 100 |
| Defined | (casualties per 1000 PAR per day). | input | | |
| WIA Rate | | | | |

Baseline WIA casualty rates based on historical data are provided for the Army and Marine Corps. Sufficient data does not exist to calculate historic ground combat WIA rates for the other services. Table 0 displays the baseline WIA rate for the Marine Corps for each troop type and battle intensity combination. Values are expressed as casualties per 1,000 PAR per day. WIA rates for combat support and service support are percentages of the combat arms WIA rate. The combat support rate is 28.5% of the combat arms rate and the service support rate is 10% of the combat arms rate. Peace Operations (Peace Ops) intensity rates are based on casualty rates from Operation New Dawn (Iraq after September 2010). Light intensity rates were derived from empirical data based on the overall average casualty rates from OEF 2010. Moderate intensity rates are derived from the average casualty rates evidenced in the Vietnam War and the Korean War. Heavy intensity rates are based on the rates seen during the Second Battle of Fallujah (during OIF; November 2004). Lastly, “Intense” battle intensity is based on rates sustained during the Battle of Hue (during the Tet Offensive in the Vietnam War).

TABLE 11
WIA Baseline Rates for U.S. Marine Corps

| Troop | | Peace | | Mod- | | |
|---|---|---|---|---|---|---|
| Type | None | ops | Light | erate | Heavy | Intense |
| Combat | 0 | 0.1000 | 0.6000 | 1.1600 | 1.8500 | 3.4700 |
| Arms | | | | | | |
| Combat | 0 | 0.0285 | 0.1710 | 0.3290 | 0.5270 | 0.9890 |
| Support | | | | | | |
| Service | 0 | 0.0100 | 0.0600 | 0.1120 | 0.1850 | 0.3470 |
| Support | | | | | | |

Table 12 displays the baseline WIA rate for the Army for each troop type and battle intensity combination. Army rates are still under development, so the Army rates are currently set to the same values as the Marine Corps rates.

TABLE 12
WIA Baseline Rates for U.S. Army

| Troop | | Peace | | | | |
|---|---|---|---|---|---|---|
| Type | None | ops | Light | Moderate | Heavy | Intense |
| Combat | 0 | 0.1000 | 0.6000 | 1.1600 | 1.8500 | 3.4700 |
| Arms | | | | | | |
| Combat | 0 | 0.0285 | 0.1710 | 0.3290 | 0.5270 | 0.9890 |
| Support | | | | | | |
| Service | 0 | 0.0100 | 0.0600 | 0.1120 | 0.1850 | 0.3470 |
| Support | | | | | | |

If the user selects the “User Defined” battle intensity, then the user defined WIA rate will be used rather than a rate from the above tables. The disease and nonbattle injury (DNBI) baseline rates are determined only by troop type, independent of battle intensity and service. Table 0 displays the three DNBI baseline rates. As with WIA rates, values are in casualties per 1,000 PAR per day.

TABLE 13
DNBI Baseline Rates

| Support category | All Intensities |
|---|---|
| Combat arms | 4.23 |
| Combat support | 3.25 |
| Service support | 3.15 |

The DNBI baseline rate calculation process produces two sets of outputs, the respective WIA and DNBI baseline rates for each user-input selection of troop type and battle intensity (if applicable).

TABLE 14
Baseline Rate Outputs

| Variable | | | | |
|---|---|---|---|---|
| name | Description | Source | Min | Max |
| BR_{WIA, Troop} | The WIA baseline | Calculate | 0 | 3.47* |
| | rate for troop | baseline rate | | |
| | type = Troop. | | | |
| BR_{DNBI, Troop} | The DNBI | Calculate | 3.15 | 4.23 |
| | baseline rate for | baseline rate | | |
| | troop type = | | | |
| | Troop. | | | |

*Max value assumes user-defined baseline WIA rate is not used.

TABLE 15
Adjustment Factor Variables

| Variable | | | | |
|---|---|---|---|---|
| name | Description | Source | Min | Max |
| BR_{WIA, Troop} | The WIA baseline rate for | Calculate | 0 | 3.47* |
| | troop type = Troop. | baseline | | |
| | | rate | | |
| BR_{DNBI, Troop} | The DNBI baseline rate for | Calculate | 3.15 | 4.23 |
| | troop type = Troop. | baseline | | |
| | | rate | | |
| rg | The region selected for the | User-input | N/A | N/A |
| | scenario rg ∈ | | | |
| | {NORTHCOM, | | | |
| | SOUTHCOM, EUCOM, | | | |
| | CENTCOM, | | | |
| | AFRICOM, PACOM} | | | |
| tr | The terrain selected for the | User-input | N/A | N/A |
| | scenario tr ∈ {Forested, | | | |
| | Mountainous, Desert, | | | |
| | Jungle, Urban} | | | |
| cl | The climate selected for the | User-input | N/A | N/A |
| | scenario cl ∈ {Hot, | | | |
| | Cold, Temperate} | | | |
| sf | The troop strength at which | User-input | 0 | 20000 |
| | the battle is adjudicated | | | |
| | for the scenario. | | | |
| NBI % | The percentage of DNBI | User-input | 0 | 100 |
| | casualties that are NBI. | | | |

*Max value assumes user-defined baseline WIA rate is not used.

The formula for adjusted casualty rates for both WIA and DNBI are:

$${WIA}_{Troop} = B {R}_{WIA , Troop} * \sqrt{r g * t r * c l * s f} and , \\ {DNBI}_{Troop} = B {R}_{DNBI , Troop} * \sqrt{NBI \% * r {g}_{NBI} + ( 1 - NBI \% ) * r {g}_{DIS}}$$

WIA Adjustment Factor for Region

Affected casualties: combat arms, combat support, and service support

CREstT allows the user to adjust the region or CCMD in which the modeled operation will occur. A previous study was performed to determine specific variables that influenced U.S. casualty incidence (Blood, Rotblatt, & Marks, 1996). The results of this study were aggregated for CCMDs during CREstT's development. Table 0 lists the adjustment factors by region.

TABLE 16
Adjustment Factors for Region

| CCMD | Adjustment factor |
|---|---|
| USNORTHCOM | 0.20 |
| USSOUTHCOM | 0.50 |
| USEUCOM | 1.31 |
| USCENTCOM | 1.03 |
| USAFRICOM | 0.92 |
| USPACOM | 1.13 |

WIA Adjustment Factor for Terrain

Affected casualties: combat arms, combat support, and service support

Previous modeling efforts by Trevor N. Dupuy (1990) have demonstrated that terrain and climate have the potential to impact the numbers of casualties in an engagement. Terrain factors previously derived by Dupuy were adapted for the development of terrain adjust factor seed in this tool. The multiplicative factors for each terrain description were averaged in the aggregated category. The “Urban” terrain type serves as the baseline value. The average factors for each category were scaled so that Urban would have a value of 1.0. Table 0 describes each of the factors used by Dupuy and the adjustment factors found in MPTk.

TABLE 17
Dupuy Terrain Values and Ajustment
factor for Terrain used in MPTk.

| Terrain Description | Dupuy | Adjustment Factor |
|---|---|---|
| Rugged | | 0.80 |
| Rugged, heavily wooded | 0.30 | |
| Rugged, mixed | 0.40 | |
| Rugged, bare | 0.50 | |
| Average | 0.40 | |
| Rolling | | 1.38 |
| Rolling, foothills, heavily wooded | 0.60 | |
| Rolling, foothills, mixed | 0.70 | |
| Rolling, foothills, bare | 0.80 | |
| Rolling, gentle, heavily wooded | 0.65 | |
| Rolling, dunes | 0.50 | |
| Rolling, gentle, mixed | 0.75 | |
| Rolling, gentle, bare | 0.85 | |
| Average | 0.69 | |
| Flat | | 1.70 |
| Flat, heavily wooded | 0.70 | |
| Flat, mixed | 0.80 | |
| Flat, bare, hard | 1.00 | |
| Flat, desert | 0.90 | |
| Average | 0.85 | |
| Swamp | | 0.70 |
| Swamp | 0.30 | |
| Swamp, mixed or open | 0.40 | |
| Average | 0.35 | |
| Urban | | 1.00 |
| Urban | 0.50 | |
| Average | 0.50 | |

WIA Adjustment Factor for Climate

Affected casualties: combat arms, combat support, and service support

Climate adjustment factors were also derived from the work of Dupuy. Climate descriptions were aggregated into larger groups similar to the process described in the Adjustment Factor for Terrain section. It should be noted that the aggregated values are adjusted so that the “Temperate” climate serves as the baseline with a value of 1. This is performed by adjusting the “Temperate” climate average to a value of 1 and adjusting each of the other aggregate values by the same multiplier.

TABLE 18
Dupuy Climat Values and Ajustment
factor for Climate used in MPTk

| Climate description | Dupuy | Adjustment factor |
|---|---|---|
| Hot | | 0.91 |
| Dry, sunshine, extreme heat | 0.8 | |
| Dry, overcast, extreme heat | 0.9 | |
| Wet, light, extreme heat | 0.7 | |
| Wet, heavy, extreme heat | 0.5 | |
| Average | 0.725 | |
| Cold | | 0.63 |
| Dry, sunshine, extreme cold | 0.7 | |
| Dry, overcast, extreme cold | 0.6 | |
| Wet, light, extreme cold | 0.4 | |
| Wet, heavy, extreme cold | 0.3 | |
| Average | 0.5 | |
| Temperate | | 1.00 |
| Dry, sunshine, temperate | 1 | |
| Dry, overcast, temperate | 1 | |
| Wet, light, temperate | 0.7 | |
| Wet, heavy, temperate | 0.5 | |
| Average | 0.8 | |

WIA Adjustment Factor for Troop Strength

Affected casualties: combat arms, combat support, and service support

The troop-strength adjustment factor is derived from the user-input unit size. However, if the unit size is greater than the PAR, the PAR will be used. Unit size will default to 1,000 unless adjusted by the user. If the user inputs a unit size of zero, the PAR will be used for the troop strength adjustment factor calculation. FIG. 5 shows changes in troop strength adjustment factor as PAR increases. Unit sizes between 869 and 19,342 are adjusted using a Weibull hazard-rate function based on the ratio of WIA rates evidenced in divisions, companies, and battalions from the Second Battle of Fallujah. The hazard-rate function is displayed in FIG. 5.

The hazard-rate step function is as follows:

$$s {f}_{us} = \{ \begin{matrix}{e}^{( - 0.0001 * 868 )} * {e}^{( 1.885438 )} & if s < 868 \\ {e}^{( - 0.0001 * us )} * {e}^{( 1.885438 )} & if 868 \leq us \leq 19341 \\ 1 & if us > 19341\end{matrix}$$

Where:
us=min(PAR, unit size)
PAR is the actual PAR for the given troop type on that day. It reflects the interval PAR decreased by casualties on previous days (unless daily replacements are enabled).

### DNBI Adjustment Factors for Region

Affected casualties: combat arms, combat support, and service support

DNBI regional adjustment factors were developed via an analysis of World War II data aggregated by both disease and NBI occurrences within each region. Disease and NBI each have an individual adjustment factor. The adjustment factors are as shown in Table 0.

TABLE 19
Regional Adjustment Factors for DNBI

| CCMD | Adjustment factor (DIS) | Adjustment factor (NBI) |
|---|---|---|
| USNORTHCOM | 1.11 | 1.09 |
| USSOUTHCOM | 1.11 | 1.09 |
| USEUCOM | 0.89 | 1.10 |
| USCENTCOM | 1.00 | 1.00 |
| USAFRICOM | 1.12 | 0.94 |
| USPACOM | 1.07 | 1.01 |

The application of the adjustment factors yields two sets of outputs: the adjusted rate for WIA casualties and the adjusted rate for DNBI casualties. Table 0 describes the outputs.

TABLE 20
Application of Adjustment Factors Outputs

| Variable | | | | |
|---|---|---|---|---|
| name | Description | Source | Min | Max |
| WIA_{Troop} | The WIA adjusted rate | Apply | 0 | 12.73* |
| | for Troop Type = Troop. | adjustment | | |
| | | factors | | |
| DNBI_{Troop} | The DNBI adjusted rate | Apply | 2.97 | 4.46 |
| | for Troop Type = Troop. | adjustment | | |
| | | factors | | |

*Max value assumes user-defined baseline WIA rate is not used.

### Generate WIA Casualties

The inputs to the WIA casualty generation process are shown in table 21 and the logic used to generate WIA casualty generation process is shown in FIG. 6.

TABLE 21
WIA Casualties Inputs

| Variable | | | | |
|---|---|---|---|---|
| name | Description | Source | Min | Max |
| WIA_{Troop} | The WIA adjusted rate | Apply | 0 | 12.73* |
| | for troop type = Troop. | adjustment | | |
| | | factors | | |
| BR_{WIA, Troop} | The WIA baseline rate | Calculate | 0 | 3.41* |
| | for troop type = Troop. | baseline | | |
| | | rate | | |
| PAR_{Troop} | The PAR for the given | User input | 0 | 500,000 |
| | troop type. | (minus | | |
| | | sustained | | |
| | | casualties) | | |
| Troop type | The troop type. Troop | User input | N/A | N/A |
| | type ε {Combat Arms, | | | |
| | Combat Support, Service | | | |
| | Support} | | | |

*Max value assumes user-defined baseline WIA rate is not used.

All CREstT casualties are generated via a mixture distribution. First, a daily rate (DailyWIAₜ) is drawn from a probability distribution that has the adjusted casualty rate (WIA_{Troop}) as its mean. As described in detail below, this distribution will be either a gamma or exponential distribution. The daily rate (DailyWIAₜ) is then applied to the current PAR and used as the mean of a Poisson distribution to generate the daily casualty count (NumWIA_{Troop}). The underlying distributions for WIA casualties are determined by the baseline WIA casualty rate (BR_{WIA,Troop}). Rates corresponding to Moderate battle intensity or lower will use a gamma distribution, while those corresponding to Heavy or above will use an exponential distribution. Table 0 displays the cutoff point between the two distributions.

TABLE 22
WIA Casualty Rate Distributions

| | Gamma | Exponential |
|---|---|---|
| Troop Type | Distribution if: | Distribution if: |
| Combat Arms | BR_{WIA, CA} < 1.505 | BR_{WIA, CA} ≥ 1.505 |
| Combat Support | BR_{WIA, CS} < 0.428 | BR_{WIA, CS} ≥ 0.428 |
| Service Support | BR_{WIA, SS} < 0.149 | BR_{WIA, SS} ≥ 0.149 |

The parameterization of the gamma distribution used in CREstT is as follows.

$$pdf \text{:} f ( x ) = \frac{1}{Γ ( α ) {β}^{a}} {x}^{α - 1} {e}^{- \frac{x}{β}}$$

Shape Parameter

$$α = \frac{{μ}^{2}}{{σ}^{2}}$$

Scale Parameter

$$β = \frac{μ}{α}$$

### Where:

μ is the mean and σ² is the variance
Γ( ) indicates the gamma function
   Random variates of the gamma distribution are calculated as follows:

Generate a random number U=uniform(0,1)

Gamma(α,β)=Gamma.Inv(U,α,β)
Where Gamma. Inv evaluates the gamma inverse cumulative distribution function at U to provide the gamma random variate.
   When generating gamma distributed casualty rates in CREstT, the mean (μ) is equal to WIA_{Troop}. It is assumed that the variance is equal to the mean to the power of 2.5. Thus, the parameters α and β can be calculated as follows:

$${σ}^{2} = {μ}^{2.5}$$

$$μ = {WIA}_{Troop}$$

$$Shape Parameter α = \frac{{μ}^{2}}{{σ}^{2}} = \frac{{μ}^{2}}{{μ}^{2.5}} = \frac{1}{\sqrt{μ}} = \frac{1}{\sqrt{{WIA}_{Troop}}}$$

$$Shape Parameter β = \frac{μ}{α} = μ * \sqrt{μ} = {μ}^{1.5} = {WIA}_{Troop}^{1.5}$$

MPTk generates gamma random variates using the acceptance-rejection method first identified by R. Cheng, as described by Law (2007).

As described above (in Table 0), heavy and intense battle intensities use the exponential distribution. The exponential distribution can be characterized as a gamma distribution with shape parameter α=1. Therefore, the parameterization of the exponential distribution is as follows:

$$pdf \text{:} f ( x ) = \frac{1}{β} {e}^{- \frac{x}{β}}$$

Where β is the mean.

Random variates of the exponential distribution are calculated as follows:
Generate a random number U=Uniform(0,1)

$$Exp ( β ) = Gamma . Inv ( U , 1 , β )$$

Where Gamma. Inv is the inverse of the gamma cumulative distribution function
When generating exponentially distributed casualty rates in CREstT, the mean (β) is equal to WIA_{Troop}.

$$β = {WIA}_{Troop}$$

For CREstT ground combat scenarios, MPTk generates exponential random variates using the same method as gamma random variates (described above) with the alpha parameter equal to 1.

### Generate Daily Casualty Rates (Combat Support and Service Support)

For combat support and service support troop types, the daily casualty rate (DailyWIAₜ) for day t is calculated by generating a random variate with mean WIA_{Troop} from either a gamma or exponential distribution using the procedures described above.
If BR_{WIA,Troop} is below cutoff (Table 0):

$$D a i {lyWIA}_{t} \text{∼} Gamma ( α = \frac{1}{\sqrt{{WIA}_{Troop}}} , β = {WIA}_{Troop}^{1.5} )$$

If BR_{WIA,Troop} is above cutoff (Table 0):

$${DailyWIA}_{t} ∼ Exp ( β = {WIA}_{Troop} )$$

### Generate Daily Casualty Rates (Combat Arms)

An underlying assumption of the CREstT casualty model is that combat arms WIA rates are autocorrelated. This autocorrelation indicates that the magnitude of any one day's casualties is related to the numbers of casualties sustained in the three immediately preceding days. Therefore, CREstT uses an autocorrelation function for the generation of combat arms casualties. Combat support and service support are not modeled using autocorrelation. The autocorrelation computation is as follows.
If BR_{WIA,Troop} is below cutoff (Table 0):

$$Dai {lyWIA}_{t} = 0.3 * ( Dai {lyWIA}_{t - 1} - μ ) + 0.2 * ( Dai {lyWIA}_{t - 2} - μ ) + 0.1 * ( Dai {lyWIA}_{t - 3} - μ ) + Gamma ( α , β )$$

Where:

$$μ = {WIA}_{Troop}$$

$$α = \frac{1}{\sqrt{{WIA}_{Troop}}}$$

$$β = {WIA}_{Troop}^{1.5}$$

If BR_{WIA,Troop} is above cutoff (Table 0):

$${DailyWIA}_{t} = 0.3 * ( {DailyWIA}_{t - 1} - μ ) + 0.2 * ( {DailyWIA}_{t - 2} - μ ) + 0.1 * ( {DailyWIA}_{t - 3} - μ ) + Exp ( β )$$

Where:

$$μ = {WIA}_{Troop} and β = {WIA}_{Troop}$$

During the first three days of the simulation (days 0, 1, and 2), casualty rates for three previous days are not available to perform the autocorrelation. This limitation is overcome by assuming that the three days prior to the start of the simulation all had rates equal to WIA_{Troop}.

$$D a i {lyWIA}_{t = - 1} = D a i {lyWIA}_{t = - 2} = D a i {lyWIA}_{t = - 3} = μ = {WIA}_{Troop}$$

This has the effect of canceling out terms in the autocorrelation equations above that do not apply. For example, on day 0 with heavy battle intensity, the autocorrelation equation would reduce to:

$$Dai {lyWIA}_{t = 0} = 0.3 * ( Dai {lyWIA}_{t = - 1} - μ ) + 0.2 * ( Dai {lyWIA}_{t = - 2} - μ ) + 0.1 * ( Dai {lyWIA}_{t = - 3} - μ ) + Exp ( β )$$

$$Dai {lyWIA}_{t = 0} = 0.3 * ( μ - μ ) + 0.2 * ( μ - μ ) + 0.1 * ( μ - μ ) + Exp ( β )$$

$$Dai {lyWIA}_{t = 0} = Exp ( β ) = Exp ( {WIA}_{Troop} )$$

It is possible for the autocorrelation equation to result in a negative result. Because casualty rates cannot be negative, negative casualty rates are corrected to 0.001 before moving on to the calculation of the next day's rate.

$$if {DailyWIA}_{t} < 0 , {DailyWIA}_{t} = 0.001$$

Once the above calculations have been performed, either in the presence or absence of autocorrelation, the resulting rate (DailyWIAₜ) is used in a Poisson distribution to generate a daily casualty estimate. The parameterization of the Poisson distribution's probability mass function is as follows:

$$pmf \text{:} f ( k ) = \frac{{λ}^{k}}{k !} {e}^{- λ}$$

Where λ is the mean.
There is no exact method for generating Poisson distributed random numbers. In MPTk, Poisson random variates with means greater than 30 are generated using the rejection method proposed by Atkinson (1979). For means less than 30, Knuth's method, as described by Law, is used (2007).

### Generate Daily Casualty Counts

To generate the daily WIA casualty estimate, the previously generated rate (DailyWIAₜ) is multiplied by the current PAR divided by 1000 and used as the mean (λ) of a Poisson distribution.

$$N {umWIA}_{Troop} = Poisson ( λ = D a i {lyWIA}_{t} * \frac{P A R}{1000} )$$

The outputs for the WIA casualty generation process are simply the number of casualties for the day that has been simulated.

TABLE 23
WIA Casualty Generation Process Outputs

| Variable | | | | |
|---|---|---|---|---|
| name | Description | Source | Min | Max |
| NumWIA_{Troop} | The number of WIA | Generate | 0 | ~30,000* |
| | casualties for troop | WIA | | |
| | type = Troop. | casualties | | |

*Max value assumes user-defined baseline WIA rate is not used.

### Generate KIA Casualties

The inputs for the KIA casualty generation process are as follows.

TABLE 24
Generate KIA Casualties Inputs

| Variable | | | | |
|---|---|---|---|---|
| Name | Description | Source | Min | Max |
| NumWIA_{Troop} | The number of WIA | Generate | 0 | ~30,000* |
| | casualties for Troop | WIA | | |
| | type = Troop. | Casualties | | |
| KIA % | The number of KIA | User-Input | 0 | 100 |
| | casualties to create as | | | |
| | a percentage of WIA | | | |
| | casualties | | | |

If the “Generate KIA Casualties” option is selected, KIA casualties are created as a percentage of the WIA casualties on each day. The calculation is as follows:

$$N {umKIA}_{Troop} = N {umWIA}_{Troop} * KIA \%$$

The number of WIA casualties is not changed when KIA casualties are created.

TABLE 25
KIA Casualty Generation Process Outputs

| Variable | | | | |
|---|---|---|---|---|
| Name | Description | Source | Min | Max |
| NumKIA_{Troop} | The number of | Generate | 0 | NumWIA_{Troop} |
| | KIA casualties for | WIA | | |
| | Troop type = | Casualties | | |
| | Troop. | | | |

Decrement the PAR after WIA and KIA

After WIA and KIA casualties have been generated, but before generating DNBI casualties, the PAR must be decremented. If the “Daily Replacements” option is selected for this troop type and interval, then the PAR is not decremented. The inputs for decrementing the PAR after WIA and KIA generation are as follows.

TABLE 26
Decrement PAR after WIA and KIA Inputs

| Variable | | | | |
|---|---|---|---|---|
| Name | Description | Source | Min | Max |
| P(WIAocc)ₓ | The probability of | PCOF | 0 | 1 |
| | occurrence of ICD-9 x | | | |
| | in the WIA PCOF | | | |
| P(Adm)ₓ | The probability that an | CREstT | 0 | 1 |
| | occurrence of ICD-9 x | common data | | |
| | becomes a theater | | | |
| | hospital admission | | | |
| PAR_{Troop} | The Population at Risk | User input | 0 | 500,000 |
| | for Troop type = | (minus | | |
| | Troop | sustained | | |
| | | casualties) | | |

If KIA casualties are generated, all KIA casualties are removed from PAR. The WIA casualties are adjusted so that only the casualties that are expected to require evacuation to Role 3 are removed. This adjustment assumes that all casualties that can return to duty after treatment at Role 1 or Role 2 are not removed from PAR and all casualties that are evacuated beyond Role 2 are permanently removed and not replaced.

$$P A {R}_{Troop} = P A {R}_{T τ o o p} - ( N {umWIA}_{Troop} * ExpEvacPerc ) - {NumKIA}_{Troop}$$

$$Where \text{:}$$

$$ExpEvacPerc = {\sum }_{x} {P ( WIAocc )}_{x} * {P ( Adm )}_{x}$$

TABLE 27
Decrement PAR after WIA and KIA Outputs

| Variable | | | | |
|---|---|---|---|---|
| Name | Description | Source | Min | Max |
| PAR_{Troop} | The Population at | Decrement PAR | 0 | 500,000 |
| | Risk for Troop type = | after WIA and | | |
| | Troop | KIA | | |

Generate DNBI Casualties

The inputs for the DNBI casualty generation process are shown in table 28.

TABLE 28
Generate DNBI Casualties Inputs

| Variable | | | | |
|---|---|---|---|---|
| name | Description | Source | Min | Max |
| DNBI_{Troop} | The DNBI adjusted rate for | Apply | 2.97 | 4.46 |
| | troop type = Troop. | adjustment | | |
| | | factors | | |
| PAR_{Troop} | The PAR for the given | User input | 0 | 500,000 |
| | troop type. | (minus | | |
| | | sustained | | |
| | | casualties) | | |
| NBI % | The percentage of DNBI | User input | 0 | 100 |
| | casualties that are NBI. | | | |

The logic to generate DNBI casualties is displayed in FIG. 7.

The underlying distribution used to create DNBI is the Weibull distribution. This distribution is standard across all troop types and battle intensities. The mean rate is the only value that changes. The parameterization for the Weibull distribution includes a shape parameter (α) and scale parameter (β). In CREstT, it is assumed that the shape parameter is 1.975658. This value is used to solve for the scale parameter. The parameterization of the Weibull distribution used in CREstT is as follows:

$$pdf = \frac{α}{β} {x}^{α - 1} {e}^{- \frac{{x}^{α}}{β}}$$

Shape Parameter α=1.975658

Scale Parameter

$$β = {( \frac{μ}{Γ ( 1 + \frac{1}{α} )} )}^{α}$$

Where:
Mean μ=DNBI_{Troop}

Γ( ) indicates the gamma function

Random variates of the Weibull distribution are calculated as follows:

Generate a random number U=uniform(0,1)

$$Weibull ( α , β ) = {( - β * ln ( U ) )}^{1 / α}$$

Thus the daily DNBI rate is:

$${DNBI}_{t} = Weibull ( α = 1.975658 , β = {( \frac{{DNBI}_{T r o o p}}{Γ ( 1 + \frac{1}{α} )} )}^{1.975658} )$$

As in the case of WIA casualties, the daily DNBI rate (DNBIₜ) is multiplied by the current PAR divided by 1000 and used as the mean (λ) of a Poisson distribution. The Poisson distribution is simulated, as described above for WIA casualties, to produce integer daily casualty counts.

$$N u m {DNBI}_{Troop} = Poission ( λ = {DNBI}_{t} * \frac{P A R}{1 , 000} )$$

CREstT generates the number of DNBI casualties per day as described above. It then splits the casualties according to the user input for “NBI % of DNBI.” The calculations are as follows:

$$N u m D i {s}_{Troop} = Round [ ( 1 - NBI \% ) * N u m {DNBI}_{Troop} ]$$

$$N u {mNBI}_{Troop} = N u m {DNBI}_{Troop} - N u m D i {s}_{Troop}$$

TABLE 29
DNBI Casualty Generation Process Outputs

| Variable | | | | |
|---|---|---|---|---|
| name | Description | Source | Min | Max |
| NumDis_{Troop} | The number of DIS | Generate | 0 | ~5000 |
| | casualties for troop | DNBI | | |
| | type = Troop. | casualties | | |
| NumNBI_{Troop} | The number of NBI | Generate | 0 | ~5000 |
| | casualties for troop | DNBI | | |
| | type = Troop. | casualties | | |

Decrement the PAR after DNBI

After DNBI casualties have been generated, but before moving to the next day, the PAR must be decremented. If the “Daily Replacements” option is selected for this troop type and interval, then the PAR is not decremented. The inputs for decrementing the PAR after DNBI generation are as follows.

TABLE 30
Decrement PAR after DNBI Inputs

| Variable | | | | |
|---|---|---|---|---|
| Name | Description | Source | Min | Max |
| P(DISocc)ₓ | The probability of | PCOF | 0 | 1 |
| | occurrence of ICD-9 x | | | |
| | in the DIS PCOF | | | |
| P(NBIocc)ₓ | The probability of | PCOF | 0 | 1 |
| | occurrence of ICD-9 x | | | |
| | in the NBI PCOF | | | |
| P(Adm)ₓ | The probability that an | CREstT | 0 | 1 |
| | occurrence of ICD-9 x | common data | | |
| | becomes a theater | | | |
| | hospital admission | | | |
| PAR_{Troop} | The Population at Risk | User input | 0 | 500,000 |
| | for Troop type = | (minus | | |
| | Troop | sustained | | |
| | | casualties) | | |

The DIS and NBI casualties are adjusted so that only the casualties that are expected to require evacuation to Role 3 are removed. This adjustment assumes that all casualties that can return to duty after treatment at Role 1 or Role 2 are not removed from PAR and all casualties that are evacuated beyond Role 2 are permanently removed and not replaced.

$$P A {R}_{Troop} = P A {R}_{Troop} - ( {NumDIS}_{Troop} * ExpDISEvacPerc ) - ( {NumNBI}_{Troop} * ExpDISEvacPerc )$$

$$Where \text{:}$$

$$ExpDISEvacPerc = {\sum }_{x} {P ( DISocc )}_{x} * {P ( Adm )}_{x}$$

$$ExpNBIEvacPerc = {\sum }_{x} {P ( NBIocc )}_{x} * {P ( Adm )}_{x}$$

TABLE 31
Decrement PAR after DNBI Outputs

| Variable | | | | |
|---|---|---|---|---|
| Name | Description | Source | Min | Max |
| PAR_{Troop} | The Population at | Decrement PAR | 0 | 500,000 |
| | Risk for Troop type = | after DNBI | | |
| | Troop | | | |

Disaster Relief

CREstT includes two modules that allow the user to develop patient streams stemming from natural disasters. These patient streams can subsequently be used to estimate the appropriate response effort. The two types of DR scenarios currently available in CREstT are earthquakes and hurricanes. The following sections provide descriptions of the overall process and describe the algorithms used in these simulations.

### Earthquake

The CREstT earthquake model estimates daily casualty composition stemming from a major earthquake. CREstT estimates the total casualty load based on user inputs for economy, population density, and the severity of the earthquake. This information is used to estimate an initial number of casualties generated by the earthquake. The user also inputs a treatment capability and day of arrival. CREstT decays the initial casualty estimate until the day of arrival. After arrival, casualties are treated each day based on the treatment capability until the mission ends. The specific workings of each subprocess are described in the following sections.

Calculate Total Casualties

The first step in the earthquake casualty generation algorithm is to calculate the total number of direct earthquake related casualties. This is a three-step process:

calculate the expected number of kills,
calculate the expected injury-to-kills ratio, and
calculate the expected number of casualties.

The inputs for these calculations are as follows.

TABLE 32
Total Earthquake Casualties Calculation Inputs

| Variable | | | | |
|---|---|---|---|---|
| name | Description | Source | Min | Max |
| Econₖᵢₗₗ | The regression coefficient for | CREstT | −6.98 | 0 |
| | number killed relative to the | common | | |
| | user-input economy. | data | | |
| PopDensₖᵢₗₗ | The regression coefficient for | CREstT | −3.50 | 0 |
| | number killed relative to the | common | | |
| | user-input population density. | data | | |
| Econᵢₙⱼ | The regression coefficient for | CREstT | −2.44 | 97.8 |
| | the injury ratio relative to the | common | | |
| | user-input economy. | data | | |
| PopDensᵢₙⱼ | The regression coefficient for | CREstT | −4.53 | 0 |
| | the injury ratio relative to the | common | | |
| | user-input population density. | data | | |
| Magnitude | The magnitude of the | User-input | 5.5 | 9.5 |
| | earthquake. | | | |

TABLE 33
Economy Regression Coefficients (Earthquake)

| Economy | Econₖᵢₗₗ | Econᵢₙⱼ |
|---|---|---|
| Developed (U.S.) | −6.9760 | 97.7946 |
| Developed (non-U.S.) | −3.3365 | −1.9408 |
| Emerging | −1 | 0 |
| Developing | 0 | −2.4355 |

TABLE 34
Population Density Regression Coefficients (Earthquake)

| Population density | PopDensₖᵢₗₗ | PopDensᵢₙⱼ |
|---|---|---|
| Low | −3.5001 | −4.5310 |
| Moderate | −3.1618 | −1.5740 |
| High | −1.8161 | −2.4978 |
| Very high | 0 | 0 |

The number of kills is calculated as follows:

$$kill = {e}^{( 8 + {Econ}_{kill} + {PopDens}_{kill} + ( Magnitude * 0.4 ) )}$$

The injury-to-kills ratio is calculated as follows:

$$InjRatio = 12 ( - 0.354 * ln ( kill ) ) + {Econ}_{inj} + {PopDens}_{inj}$$

Finally, the total number of casualties is calculated according to the following:

$$TotalCas = kill * InjRatio$$

The single output from this process is the total number of casualties.

TABLE 35
Earthquake Casualties Calculation Outputs

| Variable | | | | |
|---|---|---|---|---|
| name | Description | Source | Min | Max |
| TotalCas | The total number of | Calculate total | 105 | 717,870 |
| | casualties caused by | casualties | | |
| | the earthquake. | | | |

Decay Total Casualties until Day of Arrival

The next step in the earthquake algorithm is to calculate the number of casualties remaining on the day of arrival. The inputs into this process are as follows.

TABLE 36
Decay Casualties until Day of Arrival Inputs

| Variable | | | | |
|---|---|---|---|---|
| Name | Description | Source | Min | Max |
| TotalCas | The total number of | Calculate | 80 | 717,870 |
| | casualties caused by | total | | |
| | the earthquake | casualties | | |
| Arrival | The day that the | User-input | 0 | 180 |
| | medical treatment | | | |
| | capability begins | | | |
| | treating patients. | | | |
| lambda | Decay curve | CREstT | 0.930 | 0.995 |
| | shaping | common Data | | |
| Magnitude | The magnitude of | User-input | 5.5 | 9.5 |
| | the earthquake. | | | |

The initial number of direct earthquake casualties decreases over time. The rate at which they decrease is dependent on several unknown variables. These can include but are not limited to: the rate at which individuals stop seeking medical care; the number that die before receiving care; and the post disaster capability of the local health care system. A shaping parameter, lambda, is a proxy for these non-quantifiable effects. The model makes an assumption that a nation's economic category is closely correlated with its ability to rebuild and organize infrastructure to respond to disasters. Additionally, since larger magnitude earthquakes produce exponentially greater casualties, the model assumes that earthquakes greater than 8.1 have a slower casualty decay. Therefore, a separate lambda is provided for each economic level and magnitudes ≤8.1 and >8.1, as follows.

TABLE 37
Lambda Earthquake Values

| Economy | Magnitude | Lambda |
|---|---|---|
| Developed (US) | ≤8.1 | 0.940 |
| Developed (Non U.S.) | ≤8.1 | 0.950 |
| Emerging | ≤8.1 | 0.992 |
| Developing | ≤8.1 | 0.994 |
| Developed (US) | >8.1 | 0.930 |
| Developed (Non U.S.) | >8.1 | 0.985 |
| Emerging | >8.1 | 0.986 |
| Developing | >8.1 | 0.995 |

The calculation for the number of disaster casualties remaining i days after the earthquake, where i>0, is as follows.
The disaster casualties on day i (h0ᵢ) is initialized to the initial casualties from the earthquake (TotalCas) and the starting interval counter for the decay shaping parameter (k) is initialized to either 1 or a percentage of the initial casualties.

$$h {0}_{0} = TotalCas$$

$$k = \{ \begin{matrix}1 & if TotalCas \leq 20 , 000 \\ TotalCas * 0.001 & if totalCas > 20 , 000\end{matrix}$$

The casualties are then decayed each day using the following decay process.

$$For i = 0 to Arrival \text{-} 1 \text{:}$$

$$noise = Uniform ( - 5 , 5 )$$

$$h {0}_{( i + 1 )} = h {0}_{i} * {( lambda + delta )}^{( scaler * k + noise )}$$

$$k = k + 1$$

$$i = i + 1$$

Where

$$delta = log ( 0.5 * magnitude ) * ( 1 - lambda )$$

$$scaler = \{ \begin{matrix}log ( \frac{250 , 000}{T o t a l C a s} ) & if TotalCas \leq 250 , 000 \\ log ( 1.2 ) & if TotalCas > 250 , 000\end{matrix}$$

Delta provides an adjustment to the response based on earthquake magnitude and adds “noise” to the calculation. Scaler accelerates or decelerates the sweep as a function of the number of casualties.
   The disaster casualties remaining on the day of arrival is referred to as ArrivalCas.

$$ArrivalCas = h {0}_{arrival}$$

The outputs for this portion of the algorithm are as follows.

TABLE 38
Decay Casualties until Day of Arrival Outputs

| Variable | | | | |
|---|---|---|---|---|
| Name | Description | Source | Min | Max |
| ArrivalCas | The number of casualties | Decay | 0 | 717,870 |
| | remaining on the day of | casualties until | | |
| | arrival. | day of arrival | | |

Calculate Residual Casualties

TABLE 39
Calculate Residual Casualties Inputs

| Variable | | | | |
|---|---|---|---|---|
| Name | Description | Source | Min | Max |
| TotalCas | The total number of | Calculate total | 80 | 717,870 |
| | casualties caused by | casualties | | |
| | the earthquake | | | |

The next step in the earthquake algorithm is to calculate the residual casualties in the population. Residual casualties are diseases and traumas that are not a direct result of the earthquake event. For example, residual casualties can be injuries sustained from an automobile accident, chronic hypertension, or infectious diseases endemic in the local population. Non-disaster related casualties initially represent a small proportion of the initial causality load (Kreiss et. al., 2010). Over time the percentage of non-disaster related casualties increases until it reaches the endemic or background levels extant in the population.

The calculation for the daily number of residual casualties is:

$$ResidualCas = 1.6722 * {TotalCas}^{0.3707}$$

Generate Earthquake Casualties

TABLE 40
Calculate Residual Casualties Outputs

| Variable | | | | |
|---|---|---|---|---|
| Name | Description | Source | Min | Max |
| ResidualCas | The daily number of residual | Calculate | 8 | 248 |
| | casualties. | residual | | |
| | | casualties | | |

Beginning on the day of arrival, trauma and disease casualties are generated based on the number of initial casualties still seeking treatment and the daily number of residual casualties. After the day of arrival, casualties waiting for treatment are decayed in a manner similar to how they were decayed before they day of arrival.

TABLE 41
Generate Earthquake Casualties Inputs

| Variable | | | | |
|---|---|---|---|---|
| Name | Description | Source | Min | Max |
| TotalCas | The total number | Calculate | 80 | 717,870 |
| | of casualties caused | total | | |
| | by the earthquake | casualties | | |
| ArrivalCas | The number of | Decay | 0 | 717,870 |
| | casualties remaining | casualties | | |
| | on the day of | until day | | |
| | arrival. | of arrival | | |
| ResidualCas | The daily number of | Calculate | 8 | 248 |
| | residual casualties. | residual | | |
| | | casualties | | |
| Arrival | The day that the | User-input | 0 | 180 |
| | medical treatment | | | |
| | capability begins | | | |
| | treating patients. | | | |
| lambda | Decay curve | CREstT | 0.930 | 0.995 |
| | shaping | common | | |
| | | Data | | |
| Magnitude | The magnitude of | User input | 5.5 | 9.5 |
| | the earthquake. | | | |
| Treatment | The daily treatment | User-input | 1 | 5000 |
| | capability. | | | |
| Duration | The number of days | User-input | 1 | 180 |
| | patients will be | | | |
| | treated | | | |

The disaster casualties on day i after the earthquake (h0ᵢ) for the day of arrival is initialized to ArrivalCas and the starting interval counter for the decay shaping parameter (k) is initialized to either 5 or a percentage of the initial casualties. The delta parameter is defined in the same manner as it was before the day of arrival. The scaler parameter is defined as a function of the casualties remaining on the day of arrival (ArrivalCas).

$$h {0}_{arrival} = ArrivalCas$$

$$k = \{ \begin{matrix}5 & if h {0}_{arrival} \leq 20 , 000 \\ TotalCas * 0.001 & if h {0}_{arrival} > 20 , 000\end{matrix} \\ delta = log ( 0.5 * magnitude ) * ( 1 - lambda ) \\ scaler = \{ \begin{matrix}log ( \frac{250 , 000}{A r r i v a l C a s} ) & if Arriva l C a s \leq 250 , 000 \\ log ( 1.2 * \frac{T o t a l C a s}{A r r i v a l C a s} ) & if ArrivalCas > 250 , 000\end{matrix}$$

For each day in the casualty generation process, Trauma and Disease casualties are generated using one of three methods, depending on the number of remaining casualties, the treatment capability, and the level of residual casualties. MPTk will display results beginning with the day of arrival, which will be labeled as day zero. The trauma and disease casualties on day j after arrival (Traⱼ and Disⱼ) are calculated using the index j=i−Arrival.

$$For i = Arrival to Arrival + duration - 1 \text{:}$$

If remaining casualties (h0ᵢ) exceeds treatment capability (Treatment) then:

$${Tra}_{i \text{-} Arrival} = Poisson ( p * ( Treatment ) )$$

$${Dis}_{i \text{-} Arrival} = Poisson ( ( 1 - p ) * ( Treatment ) )$$

Where

$$p = \{ \begin{matrix}{e}^{- 0.00208 * ( ( i + 3 ) * 0.5 ) ⋀ 2.5} & if i \leq 30 \\ {e}^{- 0.00208 * ( ( 34 + \frac{i + 1}{100} ) * 0.5 ) ⋀ 2.5} & if i > 30\end{matrix}$$

If remaining casualties are less than treatment capability and ResidualCas>treatment capability then:

$$T r {a}_{i - Arrival} = Poisson ( Treatment * 0.1 )$$

$${Dis}_{i - Arrival} = Poisson ( Treatment * 0.9 )$$

If remaining casualties are less than treatment capability and ResidualCas≤treatment capability then:

$$T r {a}_{i - Arrival} = Max ( Poisson ( ResidualCas * 0.1 ) , ⌈ h {0}_{i} * p ⌉ )$$

$$Di {s}_{i - Arrival} = Max ( Poisson ( ResidualCas * 0.9 ) , ⌈ h {0}_{i} * ( 1 - p ) ⌉ )$$

Where ┌ ┐ is the ceiling operator (round up to nearest integer).
The casualties waiting for treatment on the next day is then calculated by decaying the current remaining casualties and subtracting the current day's patients.

$$noise = Uniform ( - 5 , 5 )$$

$$h {0}_{i + 1} = h {0}_{i} * {( lambda + delta )}^{( scaler * k + n o i s e )} - T r {a}_{i - Arrival} - D i {s}_{i - Arrival}$$

$$k = k + 1$$

$$i = i + 1$$

TABLE 42
Generate Earthquake Casualties Outputs

| Variable | | | | |
|---|---|---|---|---|
| name | Description | Source | Min | Max |
| Traⱼ | The number of trauma | Generate daily | 0 | ~5300 |
| | patients on day j. | casualty counts | | |
| Disⱼ | The number of disease | Generate daily | 0 | ~5300 |
| | patients on day j. | casualty counts | | |

### Hurricane

The CREstT hurricane model is similar to the earthquake model. It estimates daily casualty composition stemming from a major hurricane. Similar to the earthquake model, CREstT estimates the total casualty load based on user inputs for economy, population density, and hurricane severity. This information is used to estimate an initial casualty number. The user also inputs a treatment capability and day of arrival. CREstT decays the initial casualty estimate until the day of arrival. After arrival, casualties are treated each day based on the treatment capability until the mission ends.

Calculate Total Casualties

The first step in the hurricane casualty estimation process is to determine the total number of casualties. This process is performed in a similar fashion as described in the corresponding process in the earthquake algorithm. The steps required to perform this process are as follows:
1. calculate the expected number killed, and use the baseline fatality estimate and adjust by the population density and economic parameters to estimate the overall disaster related casualty numbers.

TABLE 43
Total Hurricane Casualties Inputs

| Variable | | | | |
|---|---|---|---|---|
| name | Description | Source | Min | Max |
| Category | The hurricane's category. | User-input | 1 | 5 |
| Econ | The average human | CREstT | 20.3 | 98.9 |
| | development index percentile | common | | |
| | rank for the user-input economy. | data | | |
| PopDens | The regression coefficient for | CREstT | 0.7 | 2.4 |
| | the user-input population density | common | | |
| | | data | | |

TABLE 44
Population Density Regression Coefficients (Hurricane)

| Population density | PopDens |
|---|---|
| Low | 0.70 |
| Moderate | 1.00 |
| High | 1.50 |
| Very high | 2.40 |

TABLE 45
Economy Regression Coefficients (Hurricane)

| Economy | Econ |
|---|---|
| Developed (U.S.) | 98.8610 |
| Developed (non-U.S.) | 82.8182 |
| Emerging | 41.5348 |
| Developing | 20.2513 |

The total number of kills is calculated as follows:

$$Kill = \{ \begin{matrix}{( 5.8 * Category - 0.085 * Econ )}^{2} * PopDens & if Category \leq 2 \\ {( 8.9 * Category - 0.171 * Econ )}^{2} * PopDens & if Category \geq 3\end{matrix}$$

The total number of casualties is calculated as follows:

$$Total Cas = Kill * 1.6 * ( 3.37 + \frac{100 - E c o n}{40} )$$

The single output from this process is the total number of expected casualties for the simulated hurricane. Table 0 describes this output.

TABLE 46
Total Hurricane Casualty Outputs

| Variable | | | | |
|---|---|---|---|---|
| name | Description | Source | Min | Max |
| TotalCas | The total number of | Calculate total | 26 | 34,686 |
| | expected casualties from | casualties. | | |
| | the hurricane. | | | |

Decay Total Casualties until Day of Arrival

The next step in the hurricane algorithm is to calculate the number of casualties remaining on the day of arrival. The inputs into this process are as follows.

TABLE 47
Decay Casualties until Day of Arrival Inputs

| Variable | | | | |
|---|---|---|---|---|
| Name | Description | Source | Min | Max |
| TotalCas | The total number of | Calculate | 26 | 34,686 |
| | casualties caused by | total | | |
| | the hurricane | casualties | | |
| Arrival | The day that the medical | User-input | 0 | 180 |
| | treatment capability | | | |
| | begins treating patients. | | | |
| lambda | Decay curve shaping | CREstT | 0.930 | 0.995 |
| | | common | | |
| | | Data | | |
| Category | The hurricane's category. | User-input | 1 | 5 |

Similar to the earthquake model, the initial number of direct disaster related casualties decreases over time. The rate at which they decrease is dependent on several unknown variables, to include but not limited to: the rate at which individuals stop seeking medical care; the number that die before receiving care; and the post disaster capability of the local health care system. A shaping parameter, lambda, is a proxy for these non-quantifiable effects. The model makes an assumption that a nation's economic category is closely correlated with its ability to rebuild and organize infrastructure to respond to disasters. Therefore, a separate lambda is provided for each economic level as follows.

TABLE 48
Hurricane Lambda Values

| Economy | Lambda |
|---|---|
| Developed (US) | 0.945 |
| Developed (Non U.S.) | 0.950 |
| Emerging | 0.970 |
| Developing | 0.980 |

The calculation for the number of disaster casualties remaining i days after the hurricane, where i>0, is as follows.
The disaster casualties on day i (h0ᵢ) is initialized to the initial casualties from the hurricane (TotalCas) and the starting interval counter for the decay shaping parameter (k) is initialized to either 5 or a percentage of the initial casualties.

$$h {0}_{0} = TotalCas$$

$$k = \{ \begin{matrix}5 & if TotalCas \leq 20 \text{,} 000 \\ TotalCas * 0.001 & if TotalCas > 20 \text{,} 000\end{matrix}$$

The casualties are then decayed each day using the following decay process.
For i=0 to Arrival-1:

$$noise = Uniform ( - 5 , 5 )$$

$$h {0}_{( i + 1 )} = h {0}_{i} * {( lambda + delta )}^{( scaler * k + n o i s e )}$$

$$k = k + 1$$

$$i = i + 1$$

Where

$$delta = log ( 0.5 * category ) * ( 1 - lambda )$$

$$scaler = \{ \begin{matrix}log ( \frac{35 , 000}{T o t a l C a s} ) & if TotalCa s \leq 20 \text{,} 000 \\ log ( 1.2 ) & if TotalCas > 20 \text{,} 000\end{matrix}$$

Delta provides an adjustment to the response based on hurricane category and adds “noise” to the calculation. Scaler accelerates or decelerates the sweep as a function of the number of casualties.
   The disaster casualties remaining on the day of arrival is referred to as ArrivalCas.

$$ArrivalCas = h {0}_{arrival}$$

The outputs for this portion of the algorithm are as follows.

TABLE 49
Decay Casualties until Day of Arrival Outputs

| Variable | | | | |
|---|---|---|---|---|
| Name | Description | Source | Min | Max |
| ArrivalCas | The number of casualties | Decay | 0 | 34,686 |
| | remaining on the day of | casualties until | | |
| | arrival. | day of arrival | | |

Calculate Residual Casualties

TABLE 50
Calculate Residual Casualties Inputs

| Variable | | | | |
|---|---|---|---|---|
| Name | Description | Source | Min | Max |
| TotalCas | The total number of | Calculate total | 26 | 34,686 |
| | casualties caused by | casualties | | |
| | the hurricane | | | |

The next step in the hurricane algorithm is to calculate the residual casualties in the population. Residual casualties are diseases and traumas that are not a direct result of the hurricane event. For example, residual casualties can be injuries sustained from an automobile accident, chronic hypertension, or infectious diseases endemic in the local population. Non-disaster related casualties initially represent a small proportion of the initial causality load (Kreiss et. al., 2010). Over time the percentage of non-disaster related casualties increases until it reaches the endemic or background levels extant in the population.

The calculation for the daily number of residual casualties is:

$$ResidualCas = 1.6722 * {TotalCas}^{0.3707}$$

TABLE 51
Calculate Residual Casualties Outputs

| Variable | | | | |
|---|---|---|---|---|
| Name | Description | Source | Min | Max |
| ResidualCas | The daily number of residual | Calculate | 6 | 81 |
| | casualties. | residual | | |
| | | casualties | | |

Generate Hurricane Casualties

Beginning on the day of arrival, trauma and disease casualties are generated based on the number of initial casualties still seeking treatment and the daily number of residual casualties. After the day of arrival, casualties waiting for treatment are decayed in a manner similar to how they were decayed before they day of arrival.

TABLE 52
Generate Hurricane Casualties Inputs

| Variable | | | | |
|---|---|---|---|---|
| Name | Description | Source | Min | Max |
| TotalCas | The total number of | Calculate | 26 | 34,686 |
| | casualties caused by | total | | |
| | the hurricane | casualties | | |
| ArrivalCas | The number of | Decay | 0 | 34,686 |
| | casualties remaining | casualties | | |
| | on the day of | until day | | |
| | arrival. | of arrival | | |
| ResidualCas | The daily number | Calculate | 6 | 81 |
| | of residual | residual | | |
| | casualties. | casualties | | |
| Arrival | The day that the | User-input | 0 | 180 |
| | medical treatment | | | |
| | capability begins | | | |
| | treating patients. | | | |
| lambda | Decay curve shaping | CREstT | 0.945 | 0.980 |
| | | common | | |
| | | Data | | |
| Category | The hurricane's | User-input | 1 | 5 |
| | category. | | | |
| Treatment | The daily treatment | User-input | 1 | 5000 |
| | capability. | | | |
| Duration | The number of days | User-input | 1 | 180 |
| | patients will be treated | | | |

The disaster casualties on day i after the hurricane (h0ᵢ) for the day of arrival is initialized to ArrivalCas and the starting interval counter for the decay shaping parameter (k) is initialized to either 5 or a percentage of the initial casualties. The delta parameter is defined in the same manner as it was before the day of arrival. The scaler parameter is defined as a function of the casualties remaining on the day of arrival (ArrivalCas).

$$h {0}_{arrival} = ArrivalCas$$

$$k = \{ \begin{matrix}5 & if h {0}_{arrival} \leq 20 \text{,} 000 \\ TotalCas * 0.001 & if h {0}_{arrival} > 20 \text{,} 000\end{matrix} \\ delta = log ( 0.5 * category ) * ( 1 - lambda ) \\ scaler = \{ \begin{matrix}log ( \frac{35 \text{,} 000}{ArrivalCas} ) & if ArrivalCas \leq 20 \text{,} 000 \\ log ( 1.2 * \frac{TotalCas}{ArrivalCas} ) & if ArrivalCas > 20 \text{,} 000\end{matrix}$$

For each day in the casualty generation process, Trauma and Disease casualties are generated using one of three methods, depending on the number of remaining casualties, the treatment capability, and the level of residual casualties. MPTk will display results beginning with the day of arrival, which will be labeled as day zero. The trauma and disease casualties on day j after arrival (Traⱼ and Disⱼ) are calculated using the index j=i−Arrival.
For

$$i = Arrival to Arrival + duration - 1 \text{:}$$

If remaining casualties (h0ᵢ) exceeds treatment capability (Treatment) then:

$$T r {a}_{i - Arrival} = Poisson ( p * ( Treatment ) )$$

$$Di {s}_{i - Arrival} = Poisson ( ( 1 - p ) * ( Treatment ) )$$

$$Where$$

$$p = \{ \begin{matrix}{e}^{- 0.0 \; 05 * ( ( i + 3 ) * 0.5 ) ⋀ 2.5} & if i \leq 20 \\ {e}^{- 0.0 \; 05 * ( ( 24 + \frac{i + 1}{100} ) * 0.5 ) ⋀ 2.5} & if i > 20\end{matrix}$$

If remaining casualties are less than treatment capability and ResidualCas>treatment capability then:

$$T r {a}_{i - Arrival} = Poisson ( Treatment * 0.1 )$$

$$D i {s}_{i - Arrival} = Poisson ( Treatment * 0.9 )$$

If remaining casualties are less than treatment capability and ResidualCas≤treatment capability then:

$$T r {a}_{i - Arrival} = Max ( Poisson ( ResidualCas * 0.1 ) , ⌈ h {0}_{i} * p ⌉ )$$

$$D i {s}_{i - Arrival} = Max ( Poisson ( ResidualCas * 0.9 ) , ⌈ h {0}_{i} * ( 1 - p ) ⌉ )$$

Where ┌ ┐ is the ceiling operator (round up to nearest integer).
The casualties waiting for treatment on the next day is then calculated by decaying the current remaining casualties and subtracting the current day's patients.

$$noise = Uniform ( - 5 , 5 )$$

$$h {0}_{i + 1} = h {0}_{i} * {( lambda + delta )}^{( scaler * k + n o i s e )} - T r {a}_{i - Arrival} - D i {s}_{i - Arrival}$$

$$k = k + 1$$

$$i = i + 1$$

TABLE 53
Generate Hurricane Casualties Outputs

| Variable | | | | |
|---|---|---|---|---|
| name | Description | Source | Min | Max |
| Traⱼ | The number of trauma | Generate daily | 0 | ~5300 |
| | patients on day j. | casualty counts | | |
| Disⱼ | The number of disease | Generate daily | 0 | ~5300 |
| | patients on day j. | casualty counts | | |

### Humanitarian Assistance

The humanitarian assistance casualty generation algorithm generates random daily casualty counts based on a user-input rate. For each interval, the inputs for this process are as follows.

TABLE 54
HA Inputs

| Variable | | | | |
|---|---|---|---|---|
| name | Description | Source | Min | Max |
| Start | The start day of the interval. | User input | 0 | 180 |
| End | The final day of the interval. | User input | 1 | 180 |
| λ | The daily rate of casualties. | User input | 1 | 5000 |
| Trauma % | The percentage of the daily | User input | 0 | 100 |
| | casualties that will be trauma. | | | |
| TransitTime | The number of days at the | User input | 0 | 179 |
| | beginning of the interval during | | | |
| | which the medical capabilities | | | |
| | are “in transit” and unable | | | |
| | to treat patients. | | | |

The first step in the HA casualty generation algorithm is to calculate the parameters of the lognormal distribution. The parameters μ and σ² are selected so that the lognormal random variates generated will have mean λ and standard deviation 0.3λ.

$$v = {( 0.3 * λ )}^{2} \\ μ = ln ( \frac{{λ}^{2}}{\sqrt{v + {λ}^{2}}} ) \\ {σ}^{2} = ln ( 1 + \frac{v}{{λ}^{2}} ) = ln ( 1.09 )$$

For each day, if the HA mission is considered “in transit”, then no casualties are produced. Otherwise, random variates are produced by first generating a lognormal random variate, then generating two Poisson random variates. The calculations are as follows for casualties on day i.

$$If i - Start < TransitTime$$

$${Trauma}_{i} = 0$$

$$D i s e a s {e}_{i} = 0$$

Otherwise

$${X}_{i} = Lognormal ( μ , {σ}^{2} )$$

$$T r a u m {a}_{i} = Poisson ( Trauma \% * {X}_{i} )$$

$$D i s e a s {e}_{i} = Poisson ( ( 1 - T r auma \% ) * {X}_{i} )$$

$$T o t a l C a s u a l t i e {s}_{i} = T r a u m {a}_{i} + D i s e a s {e}_{i}$$

Lognormal random variates are generated using an implementation of the Box-Muller transform. Poisson random variates with means greater than 30 are generated using the rejection method proposed by Atkinson (1979). For means less than 30, Knuth's method, as described by Law, is used (2007).
The outputs for this process are described in Table 0.

TABLE 55
HA Outputs

| Variable | | | | |
|---|---|---|---|---|
| name | Description | Source | Min | Max |
| TotalCasualtiesᵢ | The total number of | HA | 0 | ~15000 |
| | casualties on day i. | | | |
| Traumaᵢ | The number of trauma | HA | 0 | ~15000 |
| | casualties on day i. | | | |
| Diseaseᵢ | The number of disease | HA | 0 | ~15000 |
| | casualties on day i. | | | |

Fixed Base

The fixed base tool was designed to generate casualties resulting from various weapons used against a military base. The tool simulates a mass casualty event as a result of these attacks. Along with generating casualties, the tool also creates a patient stream based on a patient condition occurrence estimation (PCOE) developed from empirical data. This tool gives medical planners an estimate of the wounded and killed to be expected from a number of various weapon strikes.

Front End Calculations

TABLE 56
Inputs for Front-End Calculations

| Variable | | | | |
|---|---|---|---|---|
| name | Description | Source | Min | Max |
| Area_{Base} | The area of the entire | User- | >0 | 50 mi² |
| | base. | input | | |
| Area_{Units} | The units of the base area | User- | N/A | N/A |
| | Area_{Units} ∈ | input | | |
| | {Square Miles, Square | | | |
| | KM, Acre. | | | |
| LethalRadius_{radius} | The radius of weapon | User- | >0 | 300 |
| | strike i within which | input | | |
| | casualties will be | | | |
| | killed (meters). | | | |
| WoundRadius | The radius of weapon | User- | >0 | 1500 |
| | strike i within which | input | | |
| | casualties will be | | | |
| | wounded (meters). | | | |
| PAR_{Base} | The population at risk | User- | >0 | 100,000 |
| | within the entire base. | input | | |
| PercentPARⱼ | The percentage of the | User- | >0 | 100 |
| | total population at risk | input | | |
| | within sector j. | | | |
| PercentAreaⱼ | The percentage of the | User- | >0 | 100 |
| | total area of the base | input | | |
| | within sector j. | | | |

The area of the base must first be converted into square meters to simplify future calculations in which weapons are involved. These calculations are as follows:

$$If {Area}_{U n i t s} = Square Miles$$

$$Are {a}_{Base , Meters} = A r e {a}_{B a s e} * 2589975.2 \; 356$$

$$If {Area}_{U n i t s} = Square Kilometer s$$

$${Area}_{Base , Meters} = A r e {a}_{B a s e} * 1000000$$

$$If {Area}_{U n i t s} = Acres$$

$${Area}_{Base , Meters} = A r e {a}_{B a s e} * 4046.86$$

Next, TotalCasArea, LethalArea, and WoundArea must be calculated for each unique combination of WeaponType and WeaponSize.
For each weapon strike i,

$$T o t a l C a s A r e {a}_{i} = π * {( W o u n d R a d i u {s}_{i} )}^{2} \\ L e t h a l A r e {a}_{i} = π * L e t h a l R a d i u {s}_{i}^{2} \\ W o u n d A r e {a}_{i} = T o t a l C a s A r e {a}_{i} - L e t h a l A r e {a}_{i} .$$

Finally, the total area and PAR must be split amongst each of the sectors according to their characteristics. The calculations for this are as follows.

$$For each sector j \text{:}$$

$$PA {R}_{j} = P A {R}_{B a s e} * ( \frac{P e r c e n t P a {r}_{j}}{100} )$$

$${Area}_{j} = Are {a}_{B a s e} * ( \frac{P e r c e n t A r e {a}_{j}}{100} )$$

The outputs for the front end calculations are shown in 0

TABLE 57
Outputs for Front-End Calculations

| Variable | | | | |
|---|---|---|---|---|
| name | Description | Source | Min | Max |
| Area_{Base, Meters} | The area of the entire | Front end | >0 | 1.3*10⁸ |
| | base in square meters. | calculations | | |
| TotalCasAreaᵢ | The total area of weapon | Front end | >0 | 7.1*10⁶ |
| | type i within which | calculations | | |
| | casualties will be | | | |
| | wounded or killed (m²). | | | |
| LethalAreaᵢ | The area of weapon type | Front end | >0 | 282743 |
| | i within which casualties | calculations | | |
| | will be killed (m²). | | | |
| WoundAreaᵢ | The area of weapon type | Front end | >0 | 7.1*10⁶ |
| | i within which casualties | calculations | | |
| | will be wounded (m²). | | | |
| PARⱼ | The PAR within sector j. | Front end | >0 | 100000 |
| | | calculations | | |
| Areaⱼ | The area within sector j | Front end | >0 | 1.3*10⁸ |
| | (m²). | calculations | | |

Assign Hits to Sectors

The next step in the simulation process is to stochastically assign each weapon hit to individual sectors based upon their probability of being hit. The inputs for this process are shown in Table 0.

TABLE 58
Inputs for Weapon Hit Assignment

| Variable | | | | |
|---|---|---|---|---|
| name | Description | Source | Min | Max |
| PHitⱼ | The probability that a given | User | >0 | 1 |
| | weapon strike will land in sector j. | input | | |
| WeaponHitsᵢ | The number of weapon hits by | User | 1 | 100 |
| | weapon i. | input | | |

The first step in this process is to build a cumulative distribution of each of the sector's PHits. The cumulative probability for each sector is calculated according to the following:

$$C umPHi {t}_{j} = {\sum }_{k = 1}^{j} PHi {t}_{k}$$

Once a cumulative distribution has been built, weapon hits are assigned according to the following process:

$$generate a random number U = Uniform ( 0 , 1 ) , and . \qquad 2$$

select the sector from the cumulative distribution corresponding with the smallest value greater than or equal to U.
The outputs for the hit assignment process are shown in Table 0.

TABLE 59
Outputs for Weapon Hit Assignment

| Variable | | | | |
|---|---|---|---|---|
| name | Description | Source | Min | Max |
| NumHits_{i, j} | The number of hits | Assign hits | 0 | WeaponHitsᵢ |
| | from weapon type i | to sectors | | |
| | that fall within | | | |
| | sector j. | | | |

Calculate WIA and KIA

Once individual weapon hits have been assigned, the simulation calculates the number of WIA and KIA casualties for each weapon strike. The inputs for this process are shown in Table 0.

TABLE 60
Inputs for WIA and KIA Calculation

| Variable | | | | |
|---|---|---|---|---|
| name | Description | Source | Min | Max |
| NumHits_{i, j} | The number of hits | Assign | 0 | NumHitsᵢ |
| | from weapon type i | weapon hits | | |
| | that fall within | | | |
| | sector j. | | | |
| PARⱼ | The PAR within | Front end | >0 | 20000 |
| | sector j. | calculations | | |
| Areaⱼ | The area within | Front end | >0 | 1.3*10⁸ |
| | sector j. | calculations | | |
| TotalCasAreaᵢ | The total area of | Front end | >0 | 7.1*10⁶ |
| | weapon type i within | calculations | | |
| | which casualties will | | | |
| | be wounded or killed. | | | |
| LethalAreaᵢ | The area of weapon | Front end | >0 | 282743 |
| | type i within which | calculations | | |
| | casualties will be | | | |
| | killed. | | | |
| WoundAreaᵢ | The area of weapon | Front end | >0 | 7.1*10⁶ |
| | type i within which | calculations | | |
| | casualties will be | | | |
| | wounded. | | | |
| SMⱼ | The percent reduction | User-input | 0 | 100% |
| | in lethal and wounding | | | |
| | radii from shelter use. | | | |
| | SMⱼ is 0 unsheltered | | | |
| | sectors. | | | |

The calculation of KIAs and WIAs is performed according to the following.

$$If {TotalCasArea}_{i} * {( 1 - S {M}_{j} )}^{2} < {Area}_{j} :$$

$${KIA}_{j} = ( P A {R}_{j} - P A {R}_{j} * {( 1 - \frac{T o t a l C a s A r e {a}_{i} * {( 1 - S {M}_{j} )}^{2}}{A r e {a}_{j}} )}^{N u m H i t {s}_{i , j}} ) * ( \frac{L e t h a l A r e {a}_{i}}{T o t a l C a s A r e {a}_{i}} )$$

$${WIA}_{j} = ( P A {R}_{j} - P A {R}_{j} * {( 1 - \frac{T o t a l C a s A r e {a}_{i} * {( 1 - S {M}_{j} )}^{2}}{A r e {a}_{j}} )}^{N u m H i t {s}_{i , j}} ) * ( \frac{W o u n d A r e {a}_{i}}{T o t a l C a s A r e {a}_{i}} )$$

$$If {TotalCasArea}_{i} * {( 1 - S {M}_{j} )}^{2} \geq {Area}_{j}$$

$$and$$

$$LethalAre {a}_{i} * {( 1 - S {M}_{j} )}^{2} < A r e {a}_{j} \text{:}$$

$${KIA}_{j} = {( 1 - S {M}_{j} )}^{2} * P A {R}_{j} * ( \frac{L e t h a l A r e {a}_{i}}{A r e {a}_{i}} )$$

$${WIA}_{j} = P A {R}_{j} - {KIA}_{j}$$

$$If {TotalCasArea}_{i} * {( 1 - S {M}_{j} )}^{2} \geq {Area}_{j}$$

$$and$$

$$LethalAre {a}_{i} * {( 1 - S {M}_{j} )}^{2} \geq A r e {a}_{j} \text{:}$$

$${KIA}_{j} = P A {R}_{j}$$

$${WIA}_{j} = 0$$

These calculations are performed for each weapon strike, and the PAR is decremented prior to the calculations for the next weapon strike. Once all of the calculations have been performed, the total number of WIA and KIA are summed together. These are the outputs for this portion of the simulation.

TABLE 61
Outputs for WIA & KIA Calculations

| Variable | | | | |
|---|---|---|---|---|
| name | Description | Source | Min | Max |
| KIAⱼ | The number of casualties | Calculate | 0 | PARⱼ |
| | killed in action from | WIA and KIA | | |
| | sector j. | | | |
| WIAⱼ | The number of casualties | Calculate | 0 | PARⱼ |
| | wounded in action from | WIA and KIA | | |
| | sector j. | | | |
| KIA | The total number of | Calculate | 0 | PAR_{Base} |
| | casualties killed in action. | WIA and KIA | | |
| WIA | The total number of | Calculate | 0 | PAR_{Base} |
| | casualties wounded in | WIA and KIA | | |
| | action. | | | |

Shipboard

The shipboard casualty estimation tool was designed to generate casualties resulting from various weapons impacting a ship at sea. The tool, similar to the fixed base tool, generates a mass casualty event as a result of these weapon strikes. Shipboard casualty estimation tool can simulate attacks on up to five ships in one scenario. Each ship can be attacked up to five times, but it can only be attacked by one type of weapon. Each ship is simulated independently. The process below applies to a single ship and should be repeated for each ship in the scenario.

Front End Calculations

The front end calculations in shipboard calculate the WIA and KIA rate for a specific combination of ship category and weapon type. The inputs to this process are shown in the following table.

TABLE 62
Front End Calculations Inputs

| Variable | | | | |
|---|---|---|---|---|
| name | Description | Source | Min | Max |
| E[WIA]_{Class, Weapon} | The expected number of | CREstT | 2.2 | 84.0 |
| | WIA casualties when a | common data | | |
| | weapon of type Weapon hits | | | |
| | a ship of type Class. | | | |
| E[KIA]_{Class, Weapon} | The expected number of | CREstT | 1.1 | 125.0 |
| | KIA casualties when a | common data | | |
| | weapon of type Weapon hits | | | |
| | a ship of type Class. | | | |
| DefaultPAR_{Class} | The population at risk for a | CREstT | 100 | 6155 |
| | ship of type Class. | common data | | |
| Class | The category of ship class. | User input | N/A | N/A |
| | Possible values are: CVN, | | | |
| | CG/DDG/, FF/MCM/PC, | | | |
| | LHA/LHD, LSD/LPD, | | | |
| | Auxiliaries | | | |
| Weapon | The type of weapon that hits | User input | N/A | N/A |
| | the ship. Possible values are: | | | |
| | Missile, Bomb, Gunfire, | | | |
| | Torpedo, and VBIED. | | | |

The following three tables show the values of E[WIA]_{Class,Weapon}, E[KIA]_{Class,Weapon}, and DefaultPAR_{Class}. The default PAR for a CVN includes an air wing. The default PARs for other ships include ship's company, but not embarked Marines. These values are stored in the CREstT common data.

TABLE 63
Ship Types and Population at Risk

| Category | Description | PAR |
|---|---|---|
| CVN | Multi-purpose aircraft carrier | 6155 |
| CG/DDG | Guided missile cruiser, guided missile destroyer | 298 |
| FF/MCM/PC | Fast frigate, mine countermeasures ship, patrol craft | 100 |
| LHA/LHD | Amphibious assault ships | 1204 |
| LSD/LPD | Dock landing ship, amphibious transport dock | 387 |
| Auxiliaries | Auxiliary ships | 198 |

TABLE 64
Expected WIA Casualties for each Ship Class and Weapon Type

| | | | FF/MCMI | | | |
|---|---|---|---|---|---|---|
| Weapon | CVN | CG/DDG | PC | LHA/LHD | LSD/LPD | Auxiliaries |
| Missile | 49.5 | 54.4 | 14.6 | 63.1 | 31.6 | 16.4 |
| Bomb | 46.4 | 29.3 | 8.7 | 84.0 | 42.0 | 12.3 |
| Gunfire | 5.1 | 2.2 | 4.9 | 11.5 | 5.8 | 7.1 |
| Torpedo | 15.6 | 21.5 | 57.3 | 75.0 | 37.5 | 38.9 |
| Mine | 7.7 | 13.6 | 15.7 | 39.9 | 20.0 | 34.4 |
| VBIED | 39.2 | 39.0 | 44.3 | 59.7 | 34.4 | 26.5 |

Note:
VBIED is vehicle-borne improvised explosive device.

TABLE 65
Expected KIA Casualties for each Ship Class and Weapon Type

| | | | FF/MCMI | | | |
|---|---|---|---|---|---|---|
| Weapon | CVN | CG/DDG | PC | LHA/LHD | LSD/LPD | Auxiliaries |
| Missile | 40.9 | 51.1 | 7.8 | 36.2 | 18.1 | 6.0 |
| Bomb | 36.1 | 25.0 | 4.1 | 35.0 | 17.5 | 7.4 |
| Gunfire | 1.4 | 1.1 | 3.2 | 7.0 | 3.5 | 4.2 |
| Torpedo | 11.0 | 47.8 | 39.3 | 125.0 | 62.5 | 30.2 |
| Mine | 7.6 | 13.6 | 5.7 | 26.0 | 13.0 | 4.4 |
| VBIED | 11.6 | 17.0 | 11.5 | 22.5 | 13.0 | 6.3 |

Note:
VBIED is vehicle-borne improvised explosive device.

The WIA rate and KIA rate are calculated by dividing the expected number of casualties by the PAR of the ship.

$$WIARa t {e}_{Class , Weapon} = \frac{{E [ WIA ]}_{Class , Weapon}}{Defaul t P A {R}_{C l a s s}}$$

$$KIARa t {e}_{Class , Weapon} = \frac{{E [ KIA ]}_{Class , Weapon}}{Defaul t P A {R}_{Class}}$$

The outputs of this process are as follows:

TABLE 66
Front End Calculations Outputs

| Variable | | | | |
|---|---|---|---|---|
| name | Description | Source | Min | Max |
| WIARate_{Class, Weapon} | The WIA casualty rate | Front End | 0.0008 | 0.5730 |
| | (casualties per PAR) when a | Calculations | | |
| | Weapon hits a ship of type | | | |
| | Class. | | | |
| KIARate_{Class, Weapon} | The KIA casualty rate | Front End | 0.0002 | 0.3930 |
| | (casualties per PAR) when a | Calculations | | |
| | Weapon hits a ship of type | | | |
| | Class. | | | |

Casualty counts in Shipboard are generated using an exponential distribution. The parameterization of the exponential distribution is as follows:

$$pdf : f ( x ) = \frac{1}{β} {e}^{- \frac{x}{β}}$$

Where β is the mean.
Random variates of the exponential distribution are calculated as follows:
   Generate a random number U=Uniform(0,1)

$$Exp ( β ) = - β * ln ( U )$$

Calculate WIA and KIA

Once the casualty rates have been calculated, they are used to simulate the number of casualties caused by each hit. Each ship can be hit up to five times by the same type of weapon, and the PAR is decreased after each hit by removing the casualties caused by that hit. The inputs to this process are shown in the following table.

TABLE 67
Inputs for WIA and KIA Calculation

| Variable | | | | |
|---|---|---|---|---|
| name | Description | Source | Min | Max |
| WIARate_{Class, Weapon} | The WIA casualty rate | front-end | 0.0008 | 0.5730 |
| | (casualties per PAR) when a | calculations | | |
| | Weapon hits a ship of type | | | |
| | Class. | | | |
| KIARate_{Class, Weapon} | The KIA casualty rate | front-end | 0.0002 | 0.3930 |
| | (casualties per PAR) when a | calculations | | |
| | Weapon hits a ship of type | | | |
| | Class. | | | |
| NumHits | The number of times the | User input | 1 | 5 |
| | weapon hits the ship. | | | |
| PAR | The population at risk. The | User input or | 0 | 10,000 |
| | default value for the class of | CREstT | | |
| | ship will be used if a value is | common data | | |
| | not entered by the user. | | | |

The calculation of WIA and KIA casualties is performed according to the following process.
For each hit, i.
   Generate a random number of KIA and WIA casualties from an exponential distribution as described in the previous section and round the result to an integer:

$${KIA}_{i} = round ( Ex p ( β = KIARa t {e}_{Class , W e a p o n} * P A R ) )$$

$${WIA}_{i} = round ( Ex p ( β = WIARa t {e}_{Class , W e a p o n} * P A R ) )$$

If the number of KIA casualties exceeds PAR, then all PAR is KIA and there are no WIA:

$$if ( {KIA}_{i} > PAR ) \text{:}$$

$${KIA}_{i} = P A R$$

$${WIA}_{i} = 0$$

If KIA and WIA casualties combined are more than PAR, then KIA casualties are assigned first, and all remaining PAR becomes WIA:

$$if ( {KIA}_{i} + {WIA}_{i} > PAR ) \text{:}$$

$${WIA}_{i} = P A R - {KIA}_{i}$$

PAR is then decremented:

$$P A R = P A R - {KIA}_{i} - {WIA}_{i}$$

Total KIA and WIA for each ship are the sum of KIA and WIA from each hit:

$$KIA = {\sum }_{i = 1}^{N u m H i t s} {KIA}_{i}$$

$$WIA = {\sum }_{i = 1}^{N u m H i t s} {WIA}_{i}$$

The outputs for this process are as follows.

TABLE 68
Outputs for KIA and WIA Calculation

| Variable | | | | |
|---|---|---|---|---|
| name | Description | Source | Min | Max |
| KIA | The total KIA for | Calculate | 0 | PAR |
| | this ship. | WIA and KIA | | |
| WIA | The total WIA for | Calculate | 0 | PAR |
| | this ship. | WIA and KIA | | |

Assignment of ICD-9 Codes

The previous sections described the procedures used by CREstT to produce counts of casualties on a daily basis. In addition to these casualty counts, CREstT also produces patient streams, which assign ICD-9 codes to each patient. This process is common to all of the casualty generation algorithms within CREstT.

TABLE 69
Inputs for Assignment of ICD-9 Codes

| Variable | | | | |
|---|---|---|---|---|
| name | Description | Source | Min | Max |
| NumCas | Number of casualties for the | Various | 0 | PAR |
| | given day, replication, casualty | CRestT | | |
| | type, group, etc. | processes | | |
| PCOF | The PCOF selected for use with | User input | N/A | N/A |
| | these casualties. | | | |

To assign ICD-9 codes, the PCOF is first converted into a CDF (cumulative distribution function). This allows CREstT to randomly select a ICD-9 code from the distribution via the generation of a uniform (0,1) random number.

ICD-9 code assignment for each casualty consists of the following two steps:
1. generate a random number U=uniform (0,1), and
   select the ICD-9 code from the cumulative distribution corresponding with the smallest value greater than or equal to U.
The outputs of this process are an ICD-9 code assigned to each casualty.

TABLE 70
Outputs for Assignment of ICD-9 Codes

| Variable name | Description | Source |
|---|---|---|
| ICD9ᵢ | The assigned ICD-9 code | Assignment of |
| | for casualty i | ICD-9 codes |

### Combined Scenarios

Combined scenarios allow the user to combine the results of multiple individual CREstT scenarios into a single set of results. Each individual scenario is executed according to the methodology for its mission type. The combined results are then generated by treating each component scenario as its own casualty group. For mission types with multiple casualty groups, the results for the ‘Aggregate’ casualty group are sent to the combined scenario.

C. Expeditionary Medical Requirements Estimator (EMRE)

The Expeditionary Medical Requirements Estimator (EMRE) is a stochastic modelling tool that can dynamically simulate theater hospital operations. EMRE can either generate its own patient stream or import a simulated patient stream directly from CREstT. The logic diagram showing process of EMRE is shown in FIG. 8. In one embodiment, EMRE can generate its own patient stream based on the user input of an average number of patient presentations per day. EMRE first draws on a Poisson distribution to randomly generate patient numbers for each replication. The model then generates the patient stream by using that randomly drawn number of patients and a user-specified PCOF distribution. In another embodiment, if the user opts to import a CREstT-generated patient stream, EMRE randomly filters the occurrence-based casualty counts to admissions based on return-to-duty percentages. The EMRE common data tables are attached at the end of this application.

The EMRE tool is comprised of four separate algorithms:
a. the casualty generation algorithm,
b. the operation table (OT) algorithm,
c. the bed and evacuation algorithm, and
d. the blood planning factors algorithm.

### Casualty Generation

EMRE has two different methods for generating casualties: use a CREstT scenario or generate casualties using a user defined rate. In each case, MPTk will generate casualty occurrences then probabilistically determine which of those occurrences will become admissions at the theater hospitalization level of care. These two methods of generating casualties are described in detail below.

### Casualty Generation Using a CREstT Patient Stream

When a CREstT patient stream is used, all casualties from CREstT are considered. However, the patient stream generated by CREstT must be adjusted to account for the fact that many of the casualty occurrences generated by CREstT will not become admissions at the theater hospitalization level. The inputs to this process are shown in the table below.

TABLE 71
Casualty Generation Using a CREstT Patient Stream Inputs

| Variable | | | | |
|---|---|---|---|---|
| name | Description | Source | Min | Max |
| Occ_ICD9_{i, j, k} | The assigned ICD-9 code for | CREstT | N/A | N/A |
| | casualty i, rep j, day k. | | | |
| P(Adm)ₓ | The probability that an | EMRE | 0 | 100 |
| | occurrence of ICD-9 x | Common | | |
| | becomes a theater hospital | data | | |
| | admission. | | | |

The procedure for adjusting casualty occurrences to arrive at theater hospital admissions is as follows:
For each occurrence Occ_ICD9_{i,j,k}.
   Generate a Uniform(0,1) random variate, U

$$if < {P ( A d m )}_{{Occ \_ ICD 9}_{i , j , k}} , Add {Occ}_{-} ICD {9}_{i , j , k} to {ICD9}_{i , j , k}$$

Where ICD9_{i,j,k} is the ICD-9 codes for the casualties who are admitted to the theater hospital.

TABLE 72
Casualty Generation Using a CREstT
Original Patient Stream Outputs

| Variable name | Description | Source |
|---|---|---|
| ICD9_{i, j, k} | The assigned ICD-9 for | Casualty Generation Using a |
| | casualty i, rep j, day k. | CREstT Original Patient |
| | | Stream |

Casualty Generation Using a User Defined Rate
The user defined rate casualty generation process stochastically generates the number of casualties who will receive treatment at the modeled theater hospital on a given day. These numbers are distributed according to a Poisson distribution. The inputs to the user defined rate casualty generation process are shown below.

TABLE 73
Casualty Generation Using a User Defined Rate Inputs

| Variable | | | | |
|---|---|---|---|---|
| name | Description | Source | Min | Max |
| nReps | The number of replications. | User input | 1 | 200 |
| nDays | The number of days in each | User input | 1 | 180 |
| | replication. | | | |
| λ | The average number of patients | User input | 1 | 2,500 |
| | per day. | | | |
| P(Adm)ₓ | The probability that an | EMRE | 0 | 100 |
| | occurrence of ICD-9 x becomes | Common | | |
| | a theater hospital admission. | data | | |
| P(type) | The probability a theater hospital | User input | 0 | 100 |
| | admission is the given patient | | | |
| | type, where type ∈ {WIA, NBI, | | | |
| | DIS, Trauma}. | | | |
| PCOF | The user-selected distribution of | User input | N/A | N/A |
| | ICD-9 codes. | | | |

The first step when generating casualties from a user defined rate is to determine the number of admissions on each day, k, for each replication,j, (NumAdm_{j,k}). This number is determined by a random simulation of the Poisson distribution with a mean equal to the user input number of patients per day (λ). As is the case throughout MPTk, Poisson random variates with means greater than 30 are generated using the rejection method proposed by Atkinson (1979). For means less than 30, Knuth's method, as described by Law, is used (2007).

$$N u m A d {m}_{j , k} = Poisson ( λ ) ∀ j , k$$

EMRE then generates a patient stream that consists of the ICD-9 codes for each admission that occurs on each day for each replication. To accomplish this, EMRE generates casualty occurrences from the given PCOF. It then randomly determines if each occurrence becomes an admission using the same procedure used with CREstT casualty inputs in EMRE. This is repeated until the proper number of casualties has been generated (NumAdm_{j,k}). The procedure is as follows.

For each replication j and day k:

```
For n = 1 to NumAdm_{j,k}:
  Generate casualty occurrence and assign patient type
  Admission = FALSE
  While admission is FALSE
    assign ICD-9 code (Occ_ICD9_{i,j,k})
    Generate random Uniform(0,1) variate, U
    If < P (Adm)_{Occ}_ICD9_{_{i,j,k}} :
      Add Occ_ICD9_{i,j,k} to ICD9_{i,j,k}
      Admission = TRUE
    Loop
  n = n+1
```

The result of this process is the set of ICD-9 codes for every theater hospital admission on each day of each replication (ICD9_{i,j,k}). The process for generating the ICD-9 codes of casualty occurrences (Occ_ICD9_{i,j,k}) is described in detail below. EMRE first stochastically assigns the patient type of each casualty occurrence using the user-input patient type distribution (P(type)). The user-input patient type distribution is converted into a CDF (cumulative distribution function) for random selection. This allows EMRE to randomly select a patient type from the distribution via the generation of a uniform (0,1) random number. EMRE then generates a random number for each casualty and selects from the cumulative distribution. After generating a uniform (0,1) random number, EMRE selects the injury type corresponding to the smallest value greater than or equal to that number.

Injury type assignment for each casualty consists of the following two steps:
1) generate a random number U=uniform (0,1), and
2) select the injury type from the cumulative distribution corresponding with the smallest value greater than or equal to U.

Once the patient type is assigned, the casualty is randomly assigned an ICD-9 code using the user specified PCOF. The manner in which ICD-9s are assigned is identical to the process used to assign ICD-9 codes within CREstT.

TABLE 74
Casualty Generation Using a User Defined Rate Outputs

| Variable name | Description | Source |
|---|---|---|
| ICD9_{i, j, k} | The assigned ICD-9 for | Casualty Generation |
| | casualty i, rep j, day k. | Using User Defined |
| | | Rates |

Calculate Initial Surgeries

The Calculate Initial Surgeries algorithm stochastically determines whether casualties will receive surgery at the modeled theater hospital. EMRE does this based on its common data, which contains a probability of surgery value for each individual ICD-9 code. These values range from zero (in which case a particular ICD-9 code will never receive surgery) to 1 (where a casualty will always receive surgery). EMRE randomly selects from the distribution similarly to how injury types and ICD-9 codes are assigned.

TABLE 75
Calculate Initial Surgeries Inputs

| Variable | | | | |
|---|---|---|---|---|
| name | Description | Source | Min | Max |
| ICD9_{i, j, k} | The assigned ICD-9 code | ICD-9 | N/A | N/A |
| | for casualty i, rep j, day k. | assignment | | |
| | | algorithm | | |
| P(Surg)ₓ | The probability that a | EMRE | 0 | 1 |
| | patient with ICD-9 code | common | | |
| | x will receive surgery. | data | | |

Determining surgery for each casualty consists of the following two steps:
1) generate a random number U=uniform (0,1), and
2) if U≤P(Surg)ₓ, the casualty receives surgery; otherwise, they do not.

This process creates a single set of outputs-a Boolean value for each casualty describing whether they received surgery.

TABLE 76
Calculate Initial Surgeries Outputs

| Variable | | | | |
|---|---|---|---|---|
| name | Description | Source | Min | Max |
| Surg_{i, j, k} | A Boolean value for | Calculate | False = | True = |
| | whether casualty i | Initial | 0 | 1 |
| | on rep j on day k | Surgeries | | |
| | receives surgery. | | | |

These variables can be used to calculate the number of surgeries on a given day or replication. As an example, the calculation for the number of Surgeries on rep j=1 day k=1 is as follows:

$${\sum }_{i = 1}^{n} ( S u r {g}_{i , j , k} \vert j = 1 , k = 1 )$$

Calculate Follow-Up Surgeries

The logic diagram showing how follow-up surgery is calculated is shown in FIG. 9. After a casualty receives an initial surgery there is a possibility that he will require follow-up surgery. Not all patients will require follow-up surgeries. For the casualties who may receive follow-up surgery, the occurrence depends on the recurrence interval and the evacuation delay, the amount of time he is required to stay. If the casualty will require follow-up surgery before he is able to be evacuated then he will receive the surgery; otherwise, he will not. The following table describes the input variables for the follow-up surgery process.

TABLE 77
Calculate Follow-Up Surgeries Inputs

| Variable | | | | |
|---|---|---|---|---|
| name | Description | Source | Min | Max |
| ICD9_{i, j, k} | The assigned ICD-9 | ICD-9 | N/A | N/A |
| | code for casualty i, | assignment | | |
| | rep j, and day k. | algorithm | | |
| Surg_{i, j, k} | A Boolean value for | Calculate | False = | True = |
| | whether casualty i | initial | 0 | 1 |
| | on rep j on day k | surgeries | | |
| | receives surgery. | | | |
| Recurᵢ | The recurrence | EMRE | 0 | 2 |
| | interval-the time | common | | |
| | in days between | data | | |
| | the first surgery | | | |
| | and recurring | | | |
| | surgeries. | | | |
| EvacDelay | The minimum amount | User input | 1 | 4 |
| | of time, in days, | | | |
| | that a patient must | | | |
| | wait before being | | | |
| | evacuated. | | | |

TABLE 78
Calculate Follow-Up Surgeries Outputs

| Variable | | | | |
|---|---|---|---|---|
| name | Description | Source | Min | Max |
| RecurSurg_{i, j, k} | A Boolean value for | Calculate | False = | True = |
| | whether casualty i | follow-up | 0 | 1 |
| | on rep j on day k | surgeries | | |
| | receives follow-up | | | |
| | surgery. | | | |

### Calculating OR Load Hours

The next step in the EMRE process is to calculate the time in surgery for each of those casualties who required surgery in the previous two processes. EMRE's common data contains values by ICD-9 code for both initial and follow-up surgery times. If the casualty was chosen to have surgery, a value is randomly generated from a truncated normal distribution around the appropriate time. The inputs for this process are shown below.

TABLE 79
Calculate OR Load Hours Inputs

| Variable | | | | |
|---|---|---|---|---|
| name | Description | Source | Min | Max |
| ICD9_{i, j, k} | The assigned ICD-9 | ICD-9 | N/A | N/A |
| | for casualty i, rep | assignment | | |
| | j, and day k. | algorithm | | |
| Surg_{i, j, k} | A Boolean value for | Calculate | False = | True = |
| | whether casualty i | initial | 0 | 1 |
| | on rep j on day k | surgeries | | |
| | receives surgery. | | | |
| RecurSurg_{i, j, k} | A Boolean value for | Calculate | False = | True = |
| | whether casualty i | follow-up | 0 | 1 |
| | on rep j on day k | surgeries | | |
| | receives follow-up | | | |
| | surgery. | | | |
| SurgTimeₓ | The average length | EMRE | 30 | 428 |
| | of time in minutes | common | | |
| | a casualty with | data | | |
| | ICD-9 code x will | | | |
| | spend in initial | | | |
| | surgery. | | | |
| RecurTimeₓ | The average length | EMRE | 30 | 30 |
| | of time in minutes | common | | |
| | a casualty with | data | | |
| | ICD-9 code x will | | | |
| | spend in follow-up | | | |
| | surgery. | | | |
| ORSetupTime | The length of time | User input | 0 | 4 |
| | in hours required | | | |
| | to setup the OR | | | |
| | before a surgery | | | |
| | occurs. | | | |

Surgery times are drawn from a truncated normal distribution where the distribution is bounded within 20% of the mean surgical time. The standard deviation is assumed to be one fifteenth of the mean.

The total amount of OR time a patient uses for their initial surgery (ORTimeInit_{i,j,k}) is the simulated amount of time necessary to complete the surgery plus the OR setup time.

$$ORTimeIni {t}_{i , j , k} = Sur {g}_{i , j , k} * ( T r kNorm ( mean = μ , s . d . = σ , min = a , max = b ) + ORSetupTime )$$

Where:

$$μ = S u r g T i m {e}_{x} , σ = \frac{μ}{15} , a = 0.8 * μ , and b = 1. 2 * μ$$

And TrkNorm( ) is a truncated normal distribution.

A similar calculation is used to calculate the amount of OR time that is required for follow-up surgery.

$$ORTi m e R e c u {r}_{i , j , k} = Recu r S u r {g}_{i , j , k} * ( T r kNorm ( mean = μ , s . d . = σ , min = a , max = b ) + ORSetupTime )$$

Where:

$$μ = R e c u r T i m {e}_{x} , σ = \frac{μ}{15} , a = 0.8 * μ , \\ and$$

$$b = 1.2 * μ$$

And TrkNorm( ) is a truncated normal distribution.

Random variates are simulated from the truncated normal distribution as follows: The percentiles of the normal distribution that are associated with the minimum and maximum of the truncated normal distribution (p₁ and p₂) can be calculated from the CDF of the normal distribution. Because the standard deviation is a constant ratio of the mean, these values will be the same for every ICD-9 and only need to be computed once.

$${p}_{1} = N o r m . C DF ( mean = μ , s . d . = \frac{μ}{15} , x = .8 * μ ) = 0.00135$$

$${p}_{2} = N o r m . C DF ( mean = μ , s . d . = \frac{μ}{15} , x = 1. 2 * μ ) = 0.99865$$

Where Norm.CDF is the cumulative distribution function of the normal distribution evaluated at x.

To generate a random variate from this distribution, generate a uniform random number.

$$U = Uniform ( 0 , 1 )$$

Use U to generate a uniform random number between p₁ and p₂.

$$V = Uniform ( {p}_{1} , {p}_{2} ) = {p}_{1} + U * ( {p}_{2} - {p}_{1} ) = . 00135 + U * 0.9973$$

Use V to generate a normal random variate from a normal distribution.

$$T rkNor m ( μ , σ , a , b ) = Norm . In v ( x = V , mean = μ , s . d . = σ )$$

Where Norm.Inv evaluates the inverse of the Normal distribution cumulative distribution function at x.

The total number of load hours needed each day k, in a given replication j, (LoadHours_{j,k}) is the sum of the times necessary to complete all initial and follow-up surgeries that occur on that day.

$$L o a d H o u r {s}_{j , k} = {\sum }_{i} ORTimeIni {t}_{i , j , k} + {\sum }_{i} ORTi m e R e c u {r}_{i , j , k}$$

The outputs for this process are the total OR load for each day of each replication, and are described in the following table.

TABLE 80
Calculate OR Load Hours Outputs

| Variable | | | | |
|---|---|---|---|---|
| name | Description | Source | Min | Max |
| LoadHours_{j, k} | The total number of OR | Calculate OR | 0 | ∞ |
| | load hours on rep j, | load hours | | |
| | and day k. | process | | |

Calculating OR Tables

The calculation of the required number of OR tables is a simple extension of the process for calculating OR load hours. EMRE calculates, for each day, the necessary number of OR tables to handle the patient load. This calculation is based upon the following inputs.

TABLE 81
Calculate OR Tables Inputs

| Variable | | | | |
|---|---|---|---|---|
| name | Description | Source | Min | Max |
| LoadHours_{j, k} | The total number of | Calculate OR | 0 | ∞ |
| | OR load hours on | load hours | | |
| | rep j, and day k. | process | | |
| OperationalHours | The number of hours | User input | 8 | 24 |
| | each OR will be | | | |
| | operational | | | |
| | on a given day. | | | |

The calculation is the ceiling of the daily load hours divided by the operational hours. This process produces a single output—the number of required OR tables on each day of each replication

$$ORTa b l e {s}_{j , k} = ⌈ \frac{L o a d H o u r {s}_{j , k}}{Oper a t i o n a l H o u r s} ⌉$$

TABLE 82
Calculate OR Tables Outputs

| Variable | | | | |
|---|---|---|---|---|
| name | Description | Source | Min | Max |
| ORTables_{j, k} | The number of OR tables | Calculate OR | 0 | ∞ |
| | required to treat the | tables process | | |
| | patient load on rep j, | | | |
| | and day k. | | | |

Determining Patient Evac Status

The next step in the high-level EMRE process is to determine the evacuation status and length of stay in both the ICU and the ward for each patient. The inputs for this process are shown below.

TABLE 83
Determine Patient Evac Status Inputs

| Variable | | | | |
|---|---|---|---|---|
| name | Description | Source | Min | Max |
| ICD9_{i, j, k} | The assigned ICD-9 | ICD-9 | N/A | N/A |
| | code for casualty i, | assignment | | |
| | rep j, and day k. | algorithm | | |
| Surg_{i, j, k} | A Boolean value for | Calculate | False = | True = |
| | whether casualty i | initial | 0 | 1 |
| | on rep j on day k | surgeries | | |
| | receives surgery. | | | |
| ORICULOSₓ | The ICU length of | EMRE | 0 | 3 |
| | stay in days for | common | | |
| | patients with | data | | |
| | ICD-9 code x who | | | |
| | had previously | | | |
| | received surgery. | | | |
| ORWardLOSₓ | The ward length of | EMRE | 1 | 180 |
| | stay in days for | common | | |
| | patients with ICD- | data | | |
| | 9 code x who had | | | |
| | previously | | | |
| | received surgery. | | | |
| NoORICULOSₓ | The ICU length of | EMRE | 0 | 3 |
| | stay in days for | common | | |
| | patients with ICD- | data | | |
| | 9 code x who had | | | |
| | not received | | | |
| | surgery. | | | |
| NoORWardLOSₓ | The ward length of | EMRE | 1 | 180 |
| | stay in days for | common | | |
| | patients with ICD- | data | | |
| | 9 code x who had | | | |
| | not received | | | |
| | surgery. | | | |
| EvacPolicy | The maximum | User input | 3 | 15 |
| | amount of time | | | |
| | in days that | | | |
| | a casualty may | | | |
| | be held at the | | | |
| | theater hospital | | | |
| | for treatment. | | | |

There are two decision points for this logic. First, casualties are split according to whether they required surgery. Their length of stay for both the ICU and the Ward is then determined. Next, if the total length of stay is greater than the evacuation policy, the casualty will evacuate; otherwise, they will return to duty. FIG. 10 displays this logic.

As a convention, a patient's status is always determined at the end of the day. For example, a patient that arrives on day 3, stays for 3 nights in the ward, and then evacuates will generate demand for a bed on days 3, 4, and 5. On day 6, they will be counted as a ward evacuee, but they will not use a bed on day 6 because they are not present at the end of the day. The outputs for this process are as follows.

TABLE 84
Determine Patient Evac Status Outputs

| Variable | | | | |
|---|---|---|---|---|
| name | Description | Source | Min | Max |
| Status_{i, j, k} | The patient evacuation | Determine patient | Evac | RTD |
| | status for casualty i, | evacuation status | | |
| | rep j, and day k. | process | | |
| ICULOS_{i, j, k} | The ICU length of stay | Determine patient | 0 | 3 |
| | for casualty i, rep j, | evacuation status | | |
| | and day k. | process | | |
| WardLOS_{i, j, k} | The ward length of | Determine patient | 0 | 180 |
| | stay for casualty | evacuation status | | |
| | i, rep j, and day k. | process | | |

Calculating Number of Beds and Evacuations

The next step in the EMRE process is to determine the number of beds, both in the ICU and the ward, required to support the patient load on a given day. Coupled with this is the calculation of the evacuations, both from the ICU and the ward, on any given day. Casualties that evacuate from the ward are also counted towards demand for staging beds. The inputs for this process are as follows.

TABLE 85
Calculate Number of Bed and Evacuation Inputs

| Variable | | | | |
|---|---|---|---|---|
| name | Description | Source | Min | Max |
| ICD9_{i, j, k} | The assigned ICD-9 | ICD-9 | N/A | N/A |
| | for casualty, rep j, | assignment | | |
| | and day k. | algorithm | | |
| ICULOS_{i, j, k} | The ICU length of | Determine | 0 | 3 |
| | stay for casualty, | patient | | |
| | rep j, and day k. | evacuation | | |
| | | status process | | |
| WardLOS_{i, j, k} | The Ward length of | Determine | 0 | 180 |
| | stay for casualty, | patient | | |
| | rep j, and day k. | evacuation | | |
| | | status process | | |
| EvacDelay | The number of days | User input | 1 | 10 |
| | a patient must wait | | | |
| | before being | | | |
| | evacuated. | | | |
| CCATT | A Boolean value | User input | False = | True = |
| | identifying whether | | 0 | 1 |
| | CCATT teams are | | | |
| | available for | | | |
| | transport. | | | |
| StagingHold | The number of days | User input | 1 | 3 |
| | a ward evac patient | | | |
| | will be held in a | | | |
| | staging bed | | | |

This process is broken down into two subprocesses. First, the calculations are performed for casualties who were designated for evacuation in the Determining Patient Evac Status section. Next, a different process is performed for patients who were designated to return to duty. FIG. 11 and FIG. 12 outline the subprocesses. The outputs for these sub-processes include the number of beds, both in the ICU and the ward, for each day of the simulation, as well as the number of evacuations from the ICU and ward for each day.

TABLE 86
Calculate Number of Bed and Evacuation Outputs

| Variable | | | | |
|---|---|---|---|---|
| name | Description | Source | Min | Max |
| ICUBeds_{j, k} | The number of patients | Calculate beds | 0 | ∞ |
| | requiring beds in the | and evacuations | | |
| | ICU on rep j and day | process | | |
| | k. | | | |
| WardBeds_{j, k} | The number of patients | Calculate beds | 0 | ∞ |
| | requiring beds in the | and evacuations | | |
| | ward on rep j and day | process | | |
| | k. | | | |
| ICUEvacs_{j, k} | The number of patients | Calculate beds | 0 | ∞ |
| | evacuating from the | and evacuations | | |
| | ICU on rep j and day | process | | |
| | k. | | | |
| WardEvacs_{j, k} | The number of patients | Calculate beds | 0 | ∞ |
| | evacuating from the | and evacuations | | |
| | ward on rep j and day | process | | |
| | k. | | | |
| StagingBeds_{j, k} | The number of patients | Calculate beds | 0 | ∞ |
| | requiring staging beds | and evacuations | | |
| | on rep j and day k. | process | | |

Calculating Blood Planning Factors

The final process in an EMRE simulation is the calculation of blood planning factors. This process simply takes the user-input values for blood planning factors, either according to specific documentation or specific values from the user, and applies them to specific casualty types. The inputs are displayed in Table 87.

TABLE 87
Calculate Blood Planning Factors Inputs

| Variable name | Description | Source |
|---|---|---|
| CasType_{i, j, k} | The patient type for casualty i, | Casualty type |
| | rep j, and day k. | assignment |
| | | algorithm |
| RBC | The number of units of red blood | User input |
| | cells used as a planning factor | |
| | for the scenario. | |
| FFP | The number of units of fresh | User input |
| | frozen plasma used as a planning | |
| | factor for the scenario. | |
| Platelet | The number of units of platelet | User input |
| | concentrates used as a planning | |
| | factor for the scenario. | |
| Cryo | The number of units of | User input |
| | cryoprecipitate used as a planning | |
| | factor for the scenario. | |

The calculation of the blood products is simple. If a casualty has the patient type WIA, NBI, or trauma, he receives the blood products according to the user-input quantities. Therefore, it is simply a multiplier of the total number of WIA, NBI, and trauma casualties and the quantities for the blood planning factors. As an example, below is the calculation for red blood cells. The calculations for each of the other planning factors are calculated similarly.

$$R B {C}_{j , k} = R B C * ( {\sum }_{i = 1}^{n} C a s T y p {e}_{i , j , k} \vert CasType ∈ \{ WIA , NBI , Trauma \} )$$

The outputs of the calculate blood planning factors are described in Table 0.

TABLE 88
Calculate Blood Planning Factors Outputs

| Variable name | Description | Source |
|---|---|---|
| RBC_{j, k} | The number of units of red blood | User input |
| | cells required on rep j, and day k. | |
| FFP_{j, k} | The number of units of fresh | User input |
| | frozen plasma required on rep j, | |
| | and day k. | |
| Platelet_{j, k} | The number of units of platelet | User input |
| | concentrates required on rep j, | |
| | and day k. | |
| Cryo_{j, k} | The number of units of | User input |
| | cryoprecipitate required on rep j, | |
| | and day k. | |

### III. Examples of medical planning stimulations using MPTk software

The Medical Planners' Toolkit (MPTk) is a software suite of tools (modules) developed to support the joint medical planning community. This suite of tools provides planners with an end-to-end solution for medical support planning across the range of military operations (ROMO) from ground combat to humanitarian assistance. MTPk combines the Patient Condition Occurrence Frequency (PCOF) tool, the Casualty Rate Estimation Tool (CREstT), and the Expeditionary Medical Requirements Estimator (EMRE) into a single desktop application. When used individually the MPTk tools allow the user to manage the frequency distributions of probabilities of illness and injury, estimate casualties in a wide variety of military scenarios, and estimate level three theater-medical requirements. When used collectively, the tools provide medical planning data and versatility to enhance medical planners' efficiency.

The PCOF tool provides a comprehensive list of ROMO-spanning, baseline probability distributions for illness and injury based on empirical data. The tool allows users to store, edit, export, and manipulate these distributions to better fit planned operations. The PCOF tool generates precise, expected patient probability distributions. The mission-centric distributions include combat, humanitarian assistance (HR), and disaster relief (DR). These mission-centric distributions allows medical planner to assess medical risks associated with a planned mission.

The CREstT provides the capability for planners to emulate the operational plan to calculate the combat and non-combat injuries and illnesses that would be expected during military operations. Casualty estimates can be generated for ground combat, ship attacks, fixed facilities, and natural disasters. This functionality is integrated with the PCOF tool, and can use the distributions developed in that application to construct a patient stream based on the casualty estimate and user-selected PCOF distribution. CREstT uses stochastic methods to generate estimates, and can therefore provide quantile estimates in addition to average value estimates.

EMRE estimates the operating room, ICU bed, ward bed, evacuation, and blood product requirements for theater hospitalization based on a given patient load. EMRE can provide these estimates based on a user-specified average daily patient count, or it can use the patient streams derived by CREstT as EMRE is fully integrated with both CREstT and the PCOF tool. EMRE also uses stochastic processes to allow users to evaluate risk in medical planning.

The MPTk software can be used separately or collectively in medical logistics and planning. For example, the PCOF module can be used individually in a method for assessing medical risks of a planned mission comprises. The user first establishes a PCOF scenario for a planned mission. Then run simulations of the planned mission to create a set of mission-centric PCOF distributions. The PCOF stores the mission-centric PCOF distributions for presentations. The user can use these mission-centric PCOF to rank patient conditions for the mission and thus identifying medical risks for the mission.

In another embodiment, the MPTK may be used collectively in a method for assessing adequacy of a medical support plan for a mission. The user first establishes a scenario for a planned mission in MPTk. The user then stimulates the planned mission to create a set of mission-centric PCOF using PCOF module. The user then can then use the CREstT module to generate estimated estimate casualties for the planned mission and use the EMRE module to calculate estimated medical requirements for the planned mission. The results from the simulation in three modules can then be used to assess the adequacy of a medical support plan. Multiple simulations may be created and run using different user inputs, and the results from each simulation compared to select the best medical support plan, which reduces the casualty or provides adequate medical requirements for the mission. The MPTk software can also be used in a method for estimating medical requirements of a planned mission. In this embodiment, the user first establishes a scenario for a planned mission in MPTk or only in EMRE. Then the user run simulations of the planned medical support mission to generate estimated medical requirements. The estimated medical requirements may be stored and used in the planning of the mission. In an embodiment of the inventive method for estimating medical requirements medical requirements of a planned mission, medical requirements estimated including but not limited to:
a. the number of hours of operating room time needed;
b. the number of operating room tables needed;
c. the number of intensive care unit beds needed;
d. the number of ward beds needed;
e. the total number of ward and ICU beds needed;
f. the number of staging beds needed;
g. the number of patients evacuated after being treated in the ward;
h. the total number of patients evacuated from the ward and ICU;
i. the number of red blood cell units needed;
j. the number of fresh frozen plasma units needed;
k. the number of platelet concentrate units needed; and
l. the number of Cryoprecipitate units needed.

IV. Verification and Validation of MPTk Software

A MPTk V&V Working Group were designated by the Services and Combatant Commands in response to a request by The Joint Staff to support the MPTk Verification and validation effort. The members composed of medical planners from various Marine, Army, and Navy medical support commands. Each member of the Working Group received one week of MPTk training conducted at Teledyne Brown Engineering, Inc., Huntsville, Ala. The training was provided to two groups; the first group receiving training 28 Apr.-2 May 2014 and the second group from 5-9 May 2014. During the training, each member of the Working Group received training on MPTk, to include detailed instruction on the PCOF tool, CREstT, and EMRE as well as training on the verification, validation, and accreditation processes. Specific training on the V&V process included the development of acceptability criteria, testing methods, briefing formats, and the use of the Defense Health Agency's eRoom capabilities, which served as the information portal for the MPTk V&V process.

Towards the end of each week, initial testing began using the same procedures that would be used throughout the testing to familiarize each of the Working Group members with the process. The major validation events of the V&V process occurred on the Defense Connect Online (DCO), report calls that were conducted during the validation phase of the testing. On each of the DCO calls during validation testing of the model, Working Group members were presented briefings on topics they had selected on validation issues by the software developers. The Working Group members then discussed validation issues. The major issue identified during the validation phase of the testing was a recommendation to add the ability for the user to select a service baseline casualty rate (vs. a Joint baseline casualty rate) and a use redefined baseline casualty rate. The MPTk V&V Working Group members determined this was a valid concern and the capability was added to the model and thoroughly tested. Once this capability was added, the Working Group members were satisfied with the validation phase of the testing.

Comparison testing on MPTk was conducted on DCO calls on 6 Aug. 2014 and 13 Aug. 2014. Testing was conducted comparing MPTk results to real world events, and also to output from another DoD medical planning model, JMPT. Working Group members identified several issues during the comparison testing of MPTk, all of which were corrected and retested. At the conclusion of the testing, all Working Group members were satisfied with the results of the comparison testing.

Multiple iterations of the changes made have recently been incorporated into MPTk. These include:
a. Patient conditions form the basis upon which the model operates. Previous PCs were SME-derived. The patient data have been replaced with 282 single injury and 37 multiple PCs that have been developed using scientific processes and objective data.
b. A medical supply projection capability has been added that allows medical materiel to be projected for the scenarios used within the software.
c. The core data has been replaced with objective military data sets. This allows updates to be conducted on the core data files. Updating of the core data is now occurs twice annually.

Based on the foregoing, a computer system, method and software have been disclosed for medical logistic planning purpose. However, numerous modifications and substitutions can be made without deviating from the scope of the present invention. Therefore, the present invention will be disclosed, the DETAILED DESCRIPTION section, by way of example and not limitation.

### REFERENCES

1. Atkinson, A. C. (1979). Recent developments in the computer generation of Poisson random variables. Applied Statistics, 28(3), 260-263.
2. Blood, C. G., Rotblatt, D., Marks J. S. (1996). Incorporating Adversary-Specific Adjustments into the FORCAS Ground Casualty Projection Model (Report No. 96-10J). San Diego, Calif.: Naval Health Research Center.
3. Dupuy, T. N. (1990). Attrition: Forecasting battle casualties and equipment losses in modern war. Fairfax, Va.: Hero Books.
4. Elkins, T., & Wing. V. (2013). Expeditionary Medicine Requirements Estimator (EMRE) (Report No. 13-2B). San Diego, Calif.: Naval Health Research Center.
5. Elkins, T., Zouris, J., & Wing, V. (2013). The development of modules for shipboard and fixed facility casualty estimation. San Diego, Calif.: Naval Health Research Center.
6. Kreiss, Y., Merin, O., Peleg, K., Levy, G., Vinker, S., Sagi, R., & . . . Ash, N. (2010). Early disaster response in Haiti: the Israeli field hospital experience. Annals of internal medicine, 153 (1), 45-48.
7. Law, Averill M. (2007). Generating Discrete Random Variates. In K. Case & P. Wolfe (Eds.) Simulation Modeling and Analysis. (p. 466). New York: The McGraw-Hill Companies, Inc.
8. Nix, R., Negus, T. L., Elkins, T., Walker, J., Zouris, J., D'Souza, E., & Wing, V. (2013). Development of a patient condition occurrence frequency (PCOF) database for military, humanitarian assistance, and disaster relief medical data (Report No. 13-40). San Diego, Calif.: Naval Health Research Center.
9. Pan American Health Organization. (2003). Guidelines for the Use of Foreign Field Hospitals in the Aftermath of Sudden-Impact Disasters. Washington, D.C.: Regional Office of the World Health Organization.
10. Zouris, J., D'Souza, E., Elkins, T., Walker, J., Wing, V., & Brown, C. (2011). Estimation of the joint patient condition occurrence frequencies from Operation Iraqi Freedom and Operation Enduring Freedom Volume I: Development of methodology (Report No. 11-9I). San Diego, Calif.: Naval Health Research Center.
11. Zouris, J., D'Souza, E., Walker, J., Honderich, P., Tolbert, B., & Wing, V. (2013). Development of a methodology for estimating casualty occurrences and the types of illnesses and injuries for the range of military operations (Report No. 13-06). San Diego, Calif.: Naval Health Research Center.

### APPENDIX

### EMRE Common Data

The tables below (Tables 89-91) show the data used by EMRE to support the previously described processes. All variables with a source listed as “EMRE common data” are defined here. Some values may be stored at a greater precision in the MPTk database and rounded for display in these tables.

TABLE 89
EMRE Common Data: Surgery Data

| | | | | SurgTime | Recur | RecurTime |
|---|---|---|---|---|---|---|
| PC | Type | Description | P(Surg) | (mins) | (days) | (hours) |
| 005 | DMMPO | Food poisoning bacterial | 0.00 | | | 0 |
| 006 | DMMPO | Amebiasis | 0.00 | | | 0 |
| 007.9 | DMMPO | Unspecified protozoal | 0.00 | | | 0 |
| | | intestinal disease | | | | |
| 008.45 | DMMPO | Intestinal infection due to | 0.00 | | | 0 |
| | | clostridium difficile | | | | |
| 008.8 | DMMPO | Intestinal infection due to | 0.00 | | | 0 |
| | | other organism not | | | | |
| | | classified | | | | |
| 010 | DMMPO | Primary tb | 0.00 | | | 0 |
| 037 | DMMPO | Tetanus | 0.00 | | | 0 |
| 038.9 | DMMPO | Unspecified septicemia | 0.00 | | | 0 |
| 042 | DMMPO | Human immunodeficiency | 0.00 | | | 0 |
| | | virus [HIV] disease | | | | |
| 047.9 | DMMPO | Viral meningitis | 0.00 | | | 0 |
| 052 | DMMPO | Varicella | 0.00 | | | 0 |
| 053 | DMMPO | Herpes zoster | 0.00 | | | 0 |
| 054.1 | DMMPO | Genital herpes | 0.00 | | | 0 |
| 057.0 | DMMPO | Fifth disease | 0.00 | | | 0 |
| 060 | DMMPO | Yellow fever | 0.00 | | | 0 |
| 061 | DMMPO | Dengue | 0.00 | | | 0 |
| 062 | DMMPO | Mosq. borne encephalitis | 0.00 | | | 0 |
| 063.9 | DMMPO | Tick borne encephalitis | 0.00 | | | 0 |
| 065 | DMMPO | Arthropod-borne | 0.00 | | | 0 |
| | | hemorrhagic fever | | | | |
| 066.40 | DMMPO | West nile fever, | 0.00 | | | 0 |
| | | unspecified | | | | |
| 070.1 | DMMPO | Viral hepatitis | 0.00 | | | 0 |
| 071 | DMMPO | Rabies | 0.00 | | | 0 |
| 076 | DMMPO | Trachoma | 0.00 | | | 0 |
| 078.0 | DMMPO | Molluscom contagiosum | 0.00 | | | 0 |
| 078.1 | DMMPO | Viral warts | 0.00 | | | 0 |
| 078.4 | DMMPO | Hand, foot and mouth | 0.00 | | | 0 |
| | | disease | | | | |
| 079.3 | DMMPO | Rhinovirus infection in | 0.00 | | | 0 |
| | | conditions elsewhere and | | | | |
| | | of unspecified site | | | | |
| 079.99 | DMMPO | Unspecified viral infection | 0.00 | | | 0 |
| 082 | DMMPO | Tick-borne rickettsiosis | 0.00 | | | 0 |
| 084 | DMMPO | Malaria | 0.00 | | | 0 |
| 085 | DMMPO | Leishmaniasis, visceral | 0.00 | | | 0 |
| 086 | DMMPO | Trypanosomiasis | 0.00 | | | 0 |
| 091 | DMMPO | Early primary syphilis | 0.00 | | | 0 |
| 091.9 | DMMPO | Secondary syphilis, unspec | 0.00 | | | 0 |
| 094 | DMMPO | Neuro syphilis | 0.00 | | | 0 |
| 098.5 | DMMPO | Gonococcal arthritis | 0.00 | | | 0 |
| 099.4 | DMMPO | Nongonnococcal urethritis | 0.00 | | | 0 |
| 100 | DMMPO | Leptospirosis | 0.00 | | | 0 |
| 274 | DMMPO | Gout | 0.00 | | | 0 |
| 276 | DMMPO | Disorder of fluid, | 0.00 | | | 0 |
| | | electrolyte + acid base | | | | |
| | | balance | | | | |
| 296.0 | DMMPO | Bipolar disorder, single | 0.00 | | | 0 |
| | | manic episode | | | | |
| 298.9 | DMMPO | Unspecified psychosis | 0.00 | | | 0 |
| 309.0 | DMMPO | Adjustment disorder with | 0.00 | | | 0 |
| | | depressed mood | | | | |
| 309.81 | DMMPO | Ptsd | 0.00 | | | 0 |
| 309.9 | DMMPO | Unspecified adjustment | 0.00 | | | 0 |
| | | reaction | | | | |
| 310.2 | DMMPO | Post concussion syndrome | 0.00 | | | 0 |
| 345.2 | DMMPO | Epilepsy petit mal | 0.00 | | | 0 |
| 345.3 | DMMPO | Epilepsy grand mal | 0.00 | | | 0 |
| 346 | DMMPO | Migraine | 0.00 | | | 0 |
| 361 | DMMPO | Retinal detachment | 0.00 | | | 0 |
| 364.3 | DMMPO | Uveitis nos | 0.00 | | | 0 |
| 365 | DMMPO | Glaucoma | 0.00 | | | 0 |
| 370.0 | DMMPO | Corneal ulcer | 0.00 | | | 0 |
| 379.31 | DMMPO | Aphakia | 0.00 | | | 0 |
| 380.1 | DMMPO | Infective otitis externa | 0.00 | | | 0 |
| 380.4 | DMMPO | Impacted cerumen | 0.00 | | | 0 |
| 381 | DMMPO | Acute nonsuppurative | 0.00 | | | 0 |
| | | otitis media | | | | |
| 381.9 | DMMPO | Unspecified eustachian | 0.00 | | | 0 |
| | | tube disorder | | | | |
| 384.2 | DMMPO | Perforated tympanic | 0.00 | | | 0 |
| | | membrane | | | | |
| 388.3 | DMMPO | Tinnitus, unspecified | 0.00 | | | 0 |
| 389.9 | DMMPO | Unspecified hearing loss | 0.00 | | | 0 |
| 401 | DMMPO | Essential hypertension | 0.00 | | | 0 |
| 410 | DMMPO | Myocardial infarction | 0.00 | | | 0 |
| 413.9 | DMMPO | Other and unspecified | 0.00 | | | 0 |
| | | angina pectoris | | | | |
| 427.9 | DMMPO | Cardiac dysryhthmia | 0.00 | | | 0 |
| | | unspecified | | | | |
| 453.4 | DMMPO | Venous | 0.00 | | | 0 |
| | | embolism/thrombus of | | | | |
| | | deep vessels lower | | | | |
| | | extremity | | | | |
| 462 | DMMPO | Acute pharyngitis | 0.00 | | | 0 |
| 465 | DMMPO | Acute uri of multiple or | 0.00 | | | 0 |
| | | unspecified sites | | | | |
| 466 | DMMPO | Acute bronchitis & | 0.00 | | | 0 |
| | | bronchiolitis | | | | |
| 475 | DMMPO | Peritonsillar abscess | 0.25 | 176 | | 0 |
| 486 | DMMPO | Pneumonia, organism | 0.00 | | | 0 |
| | | unspecified | | | | |
| 491 | DMMPO | Chronic bronchitis | 0.00 | | | 0 |
| 492 | DMMPO | Emphysema | 0.00 | | | 0 |
| 493.9 | DMMPO | Asthma | 0.00 | | | 0 |
| 523 | DMMPO | Gingival and periodontal | 0.00 | | | 0 |
| | | disease | | | | |
| 530.2 | DMMPO | Ulcer of esophagus | 0.00 | | | 0 |
| 530.81 | DMMPO | Gastroesophageal reflux | 0.00 | | | 0 |
| 531 | DMMPO | Gastric ulcer | 0.00 | | | 0 |
| 532 | DMMPO | Duodenal ulcer | 0.18 | 150 | | 0 |
| 540.9 | DMMPO | Acute appendicitis without | 0.80 | 291 | 1 | 0.5 |
| | | mention of peritonitis | | | | |
| 541 | DMMPO | Appendicitis, unspecified | 0.83 | 90 | 1 | 0.5 |
| 550.9 | DMMPO | Unilateral inguinal hernia | 0.01 | 191 | | 0 |
| 553.1 | DMMPO | Umbilical hernia | 0.87 | 90 | | 0 |
| 553.9 | DMMPO | Hernia nos | 0.10 | 90 | | 0 |
| 564.0 | DMMPO | Constipation | 0.00 | | | 0 |
| 564.1 | DMMPO | Irritable bowel disease | 0.00 | | | 0 |
| 566 | DMMPO | Abscess of anal and rectal | 0.75 | 45 | 1 | 0.5 |
| | | regions | | | | |
| 567.9 | DMMPO | Unspecified peritonitis | 0.00 | | | 0 |
| 574 | DMMPO | Cholelithiasis | 0.05 | 182 | | 0 |
| 577.0 | DMMPO | Acute pancreatitis | 0.00 | | | 0 |
| 577.1 | DMMPO | Chronic pancreatitis | 0.00 | | | 0 |
| 578.9 | DMMPO | Hemorrhage of | 0.00 | | | 0 |
| | | gastrointestinal tract | | | | |
| | | unspecified | | | | |
| 584.9 | DMMPO | Acute renal failure | 0.00 | | | 0 |
| | | unspecified | | | | |
| 592 | DMMPO | Calculus of kidney | 0.00 | | | 0 |
| 599.0 | DMMPO | Unspecified urinary tract | 0.00 | | | 0 |
| | | infection | | | | |
| 599.7 | DMMPO | Hematuria | 0.00 | | | 0 |
| 608.2 | DMMPO | Torsion of testes | 1.00 | 147 | | 0 |
| 608.4 | DMMPO | Other inflammatory | 0.00 | | | 0 |
| | | disorders of male genital | | | | |
| | | organs | | | | |
| 611.7 | DMMPO | Breast lump | 0.00 | | | 0 |
| 633 | DMMPO | Ectopic preg | 0.50 | 173 | | 0 |
| 634 | DMMPO | Spontaneous abortion | 0.75 | 162 | | 0 |
| 681 | DMMPO | Cellulitis and abscess of | 0.00 | | | 0 |
| | | finger and toe | | | | |
| 682.0 | DMMPO | Cellulitis and abscess of | 0.00 | | | 0 |
| | | face | | | | |
| 682.6 | DMMPO | Cellulitis and abscess of | 0.00 | | | 0 |
| | | leg except foot | | | | |
| 682.7 | DMMPO | Cellulitis and abscess of | 0.00 | | | 0 |
| | | foot except toes | | | | |
| 682.9 | DMMPO | Cellulitis and abscess of | 0.00 | | | 0 |
| | | unspecified parts | | | | |
| 719.41 | DMMPO | Pain in joint shoulder | 0.00 | | | 0 |
| 719.46 | DMMPO | Pain in joint lower leg | 0.00 | | | 0 |
| 719.47 | DMMPO | Pain in joint ankle/foot | 0.00 | | | 0 |
| 722.1 | DMMPO | Displacement lumbar | 0.00 | | | 0 |
| | | intervertebral disc w/o | | | | |
| | | myelopathy | | | | |
| 723.0 | DMMPO | Spinal stenosis in cervical | 0.00 | | | 0 |
| | | region | | | | |
| 724.02 | DMMPO | Spinal stenosis of lumbar | 0.00 | | | 0 |
| | | region | | | | |
| 724.2 | DMMPO | Lumbago | 0.00 | | | 0 |
| 724.3 | DMMPO | Sciatica | 0.00 | | | 0 |
| 724.4 | DMMPO | Lumbar sprain | 0.00 | | | 0 |
| | | (thoracic/lumbosacral) | | | | |
| | | neuritis or radiculitis, | | | | |
| | | unspec | | | | |
| 724.5 | DMMPO | Backache unspecified | 0.00 | | | 0 |
| 726.10 | DMMPO | Disorders of bursae and | 0.00 | | | 0 |
| | | tendons in shoulder | | | | |
| | | unspecified | | | | |
| 726.12 | DMMPO | Bicipital tenosynovitis | 0.00 | | | 0 |
| 726.3 | DMMPO | Enthesopathy of elbow | 0.00 | | | 0 |
| | | region | | | | |
| 726.4 | DMMPO | Enthesopathy of wrist and | 0.00 | | | 0 |
| | | carpus | | | | |
| 726.5 | DMMPO | Enthesopathy of hip region | 0.00 | | | 0 |
| 726.6 | DMMPO | Enthesopathy of knee | 0.00 | | | 0 |
| 726.7 | DMMPO | Enthesopathy of ankle and | 0.00 | | | 0 |
| | | tarsus | | | | |
| 729.0 | DMMPO | Rheumatism unspecified | 0.00 | | | 0 |
| | | and fibrositis | | | | |
| 729.5 | DMMPO | Pain in limb | 0.00 | | | 0 |
| 780.0 | DMMPO | Alterations of | 0.00 | | | 0 |
| | | consciousness | | | | |
| 780.2 | DMMPO | Syncope | 0.00 | | | 0 |
| 780.39 | DMMPO | Other convulsions | 0.00 | | | 0 |
| 780.5 | DMMPO | Sleep disturbances | 0.00 | | | 0 |
| 780.6 | DMMPO | Fever | 0.00 | | | 0 |
| 782.1 | DMMPO | Rash and other nonspecific | 0.00 | | | 0 |
| | | skin eruptions | | | | |
| 782.3 | DMMPO | Edema | 0.00 | | | 0 |
| 783.0 | DMMPO | Anorexia | 0.00 | | | 0 |
| 784.0 | DMMPO | Headache | 0.00 | | | 0 |
| 784.7 | DMMPO | Epistaxis | 0.00 | | | 0 |
| 784.8 | DMMPO | Hemorrhage from throat | 0.00 | | | 0 |
| 786.5 | DMMPO | Chest pain | 0.00 | | | 0 |
| 787.0 | DMMPO | Nausea and vomiting | 0.00 | | | 0 |
| 787.91 | DMMPO | Diarrhea nos | 0.00 | | | 0 |
| 789.00 | DMMPO | Abdominal pain | 0.00 | | | 0 |
| | | unspecified site | | | | |
| 800.0 | DMMPO | Closed fracture of vault of | 0.00 | | | 0 |
| | | skull without intracranial | | | | |
| | | injury | | | | |
| 801.0 | DMMPO | Closed fracture of base of | 0.10 | 200 | | 0 |
| | | skull without intracranial | | | | |
| | | injury | | | | |
| 801.76 | DMMPO | Open fracture base of skull | 1.00 | 241 | | 0 |
| | | with subarachnoid, | | | | |
| | | subdural and extradural | | | | |
| | | hemorrhage with loss of | | | | |
| | | consciousness of | | | | |
| | | unspecified duration | | | | |
| 802.0 | DMMPO | Closed fracture of nasal | 0.10 | 211 | | 0 |
| | | bones | | | | |
| 802.1 | DMMPO | Open fracture of nasal | 1.00 | 241 | | 0 |
| | | bones | | | | |
| 802.6 | DMMPO | Fracture orbital floor | 0.30 | 179 | | 0 |
| | | closed (blowout) | | | | |
| 802.7 | DMMPO | Fracture orbital floor open | 1.00 | 241 | | 0 |
| | | (blowout) | | | | |
| 802.8 | DMMPO | Closed fracture of other | 0.10 | 192 | | 0 |
| | | facial bones | | | | |
| 802.9 | DMMPO | Open fracture of other | 1.00 | 241 | | 0 |
| | | facial bones | | | | |
| 805 | DMMPO | Closed fracture of cervical | 0.35 | 180 | | 0 |
| | | vertebra w/o spinal cord | | | | |
| | | injury | | | | |
| 806.1 | DMMPO | Open fracture of cervical | 0.15 | 212 | | 0 |
| | | vertebra with spinal cord | | | | |
| | | injury | | | | |
| 806.2 | DMMPO | Closed fracture of dorsal | 0.10 | 201 | | 0 |
| | | vertebra with spinal cord | | | | |
| | | injury | | | | |
| 806.3 | DMMPO | Open fracture of dorsal | 0.40 | 242 | | 0 |
| | | vertebra with spinal cord | | | | |
| | | injury | | | | |
| 806.4 | DMMPO | Closed fracture of lumbar | 0.25 | 200 | | 0 |
| | | spine with spinal cord | | | | |
| | | injury | | | | |
| 806.5 | DMMPO | Open fracture of lumbar | 1.00 | 241 | | 0 |
| | | spine with spinal cord | | | | |
| | | injury | | | | |
| 806.60 | DMMPO | Closed fracture sacrum | 0.25 | 200 | | 0 |
| | | and coccyx w/unspec. | | | | |
| | | spinal cord injury | | | | |
| 806.70 | DMMPO | Open fracture sacrum and | 1.00 | 241 | | 0 |
| | | coccyx w/unspec. spinal | | | | |
| | | cord injury | | | | |
| 807.0 | DMMPO | Closed fracture of rib(s) | 0.10 | 60 | | 0 |
| 807.1 | DMMPO | Open fracture of rib(s) | 1.00 | 284 | 1 | 0.5 |
| 807.2 | DMMPO | Closed fracture of sternum | 0.10 | 200 | | 0 |
| 807.3 | DMMPO | Open fracture of sternum | 1.00 | 241 | | 0 |
| 808.8 | DMMPO | Fracture of pelvis | 0.95 | 313 | | 0 |
| | | unspecified, closed | | | | |
| 808.9 | DMMPO | Fracture of pelvis | 1.00 | 329 | | 0 |
| | | unspecified, open | | | | |
| 810.0 | DMMPO | Clavicle fracture, closed | 0.35 | 45 | | 0 |
| 810.1 | DMMPO | Clavicle fracture, open | 1.00 | 241 | | 0 |
| 810.12 | DMMPO | Open fracture of shaft of | 1.00 | 241 | 1 | 0.5 |
| | | clavicle | | | | |
| 811.0 | DMMPO | Fracture of scapula, closed | 0.10 | 200 | | 0 |
| 811.1 | DMMPO | Fracture of scapula, open | 1.00 | 241 | 1 | 0.5 |
| 812.00 | DMMPO | Fracture of unspecified | 0.25 | 200 | | 0 |
| | | part of upper end of | | | | |
| | | humerus, closed | | | | |
| 813.8 | DMMPO | Fracture unspecified part | 0.25 | 200 | | 0 |
| | | of radius and ulna closed | | | | |
| 813.9 | DMMPO | Fracture unspecified part | 1.00 | 256 | 1 | 0.5 |
| | | of radius and ulna open | | | | |
| 815.0 | DMMPO | Closed fracture of | 0.10 | 211 | | 0 |
| | | metacarpal bones | | | | |
| 816.0 | DMMPO | Phalanges fracture, closed | 0.10 | 211 | | 0 |
| 816.1 | DMMPO | Phalanges fracture, open | 1.00 | 84 | 1 | 0.5 |
| 817.0 | DMMPO | Multiple closed fractures | 0.10 | 68 | | 0 |
| | | of hand bones | | | | |
| 817.1 | DMMPO | Multiple open fracture of | 1.00 | 86 | 1 | 0.5 |
| | | hand bones | | | | |
| 820.8 | DMMPO | Fracture of femur neck, | 0.25 | 200 | | 0 |
| | | closed | | | | |
| 820.9 | DMMPO | Fracture of femur neck, | 1.00 | 241 | 1 | 0.5 |
| | | open | | | | |
| 821.01 | DMMPO | Fracture shaft femur, | 1.00 | 208 | | 0 |
| | | closed | | | | |
| 821.11 | DMMPO | Fracture shaft of femur, | 1.00 | 238 | 1 | 0.5 |
| | | open | | | | |
| 822.0 | DMMPO | Closed fracture of patella | 0.25 | 200 | | 0 |
| 822.1 | DMMPO | Open fracture of patella | 1.00 | 229 | 1 | 0.5 |
| 823.82 | DMMPO | Fracture fib fib, closed | 0.25 | 233 | | 0 |
| 823.9 | DMMPO | Fracture of unspecified | 1.00 | 258 | 1 | 0.5 |
| | | part of tibia and fibula | | | | |
| | | open | | | | |
| 824.8 | DMMPO | Fracture ankle, nos, closed | 0.25 | 222 | | 0 |
| 824.9 | DMMPO | Ankle fracture, open | 1.00 | 251 | 1 | 0.5 |
| 825.0 | DMMPO | Fracture to calcaneus, | 0.25 | 200 | | 0 |
| | | closed | | | | |
| 826.0 | DMMPO | Closed fracture of one or | 0.10 | 211 | | 0 |
| | | more phalanges of foot | | | | |
| 829.0 | DMMPO | Fracture of unspecified | 0.25 | 200 | | 0 |
| | | bone, closed | | | | |
| 830.0 | DMMPO | Closed dislocation of jaw | 0.00 | | | 0 |
| 830.1 | DMMPO | Open dislocation of jaw | 0.10 | 235 | 1 | 0.5 |
| 831 | DMMPO | Dislocation shoulder | 0.00 | | | 0 |
| 831.04 | DMMPO | Closed dislocation of | 0.00 | | | 0 |
| | | acromioclavicular joint | | | | |
| 831.1 | DMMPO | Dislocation of shoulder, | 0.10 | 235 | 1 | 0.5 |
| | | open | | | | |
| 832.0 | DMMPO | Dislocation elbow, closed | 0.00 | | | 0 |
| 832.1 | DMMPO | Dislocation elbow, open | 0.10 | 235 | 1 | 0.5 |
| 833 | DMMPO | Dislocation wrist closed | 0.45 | 120 | | 0 |
| 833.1 | DMMPO | Dislocated wrist, open | 0.45 | 235 | 1 | 0.5 |
| 834.0 | DMMPO | Dislocation of finger, | 0.00 | | | 0 |
| | | closed | | | | |
| 834.1 | DMMPO | Dislocation of finger, open | 0.10 | 235 | 1 | 0.5 |
| 835 | DMMPO | Closed dislocation of hip | 0.00 | | | 0 |
| 835.1 | DMMPO | Hip dislocation open | 0.45 | 235 | | 0 |
| 836.0 | DMMPO | Medial meniscus tear | 0.00 | | | 0 |
| 836.1 | DMMPO | Lateral meniscus tear | 0.00 | | | 0 |
| 836.2 | DMMPO | Meniscus tear of knee | 0.00 | | | 0 |
| 836.5 | DMMPO | Dislocation knee, closed | 0.00 | | | 0 |
| 836.6 | DMMPO | Other dislocation of knee | 0.45 | 235 | 1 | 0.5 |
| | | open | | | | |
| 839.01 | DMMPO | Closed dislocation first | 0.00 | | | 0 |
| | | cervical vertebra | | | | |
| 840.4 | DMMPO | Rotator cuff sprain | 0.00 | | | 0 |
| 840.9 | DMMPO | Sprain shoulder | 0.00 | | | 0 |
| 843 | DMMPO | Sprains and strains of hip | 0.00 | | | 0 |
| | | and thigh | | | | |
| 844.9 | DMMPO | Sprain, knee | 0.00 | | | 0 |
| 845 | DMMPO | Sprain of ankle | 0.00 | | | 0 |
| 846 | DMMPO | Sprains and strains of | 0.00 | | | 0 |
| | | socrmliac region | | | | |
| 846.0 | DMMPO | Sprain of lumbosacral | 0.00 | | | 0 |
| | | (joint) (ligament) | | | | |
| 847.2 | DMMPO | Sprain lumbar region | 0.00 | | | 0 |
| 847.3 | DMMPO | Sprain of sacrum | 0.00 | | | 0 |
| 848.1 | DMMPO | Jaw sprain | 0.00 | | | 0 |
| 848.3 | DMMPO | Sprain of ribs | 0.00 | | | 0 |
| 850.9 | DMMPO | Concussion | 0.00 | | | 0 |
| 851.0 | DMMPO | Cortex (Cerebral) | 0.00 | | | 0 |
| | | contusion w/o open | | | | |
| | | intracranial wound | | | | |
| 851.01 | DMMPO | Cortex (Cerebral) | 0.00 | | | 0 |
| | | contusion w/o open wound | | | | |
| | | no loss of consciousness | | | | |
| 852 | DMMPO | Subarachnoid subdural | 0.15 | 338 | | 0 |
| | | extradural hemorrhage | | | | |
| | | injury | | | | |
| 853 | DMMPO | Other and unspecified | 0.15 | 335 | | 0 |
| | | intracranial hemorrhage | | | | |
| | | injury w/o open wound | | | | |
| 853.15 | DMMPO | Unspecified intracranial | 0.15 | 337 | 1 | 0.5 |
| | | hemorrhage with open | | | | |
| | | intracranial wound | | | | |
| 860.0 | DMMPO | Traumatic pneumothorax | 0.30 | 250 | | 0 |
| | | w/o open wound into | | | | |
| | | thorax | | | | |
| 860.1 | DMMPO | Traumatic pneumothorax | 0.30 | 250 | 1 | 0.5 |
| | | w/open wound into thorax | | | | |
| 860.2 | DMMPO | Traumatic hemothorax w/o | 0.30 | 250 | | 0 |
| | | open wound into thorax | | | | |
| 860.3 | DMMPO | Traumatic hemothorax | 0.30 | 250 | 1 | 0.5 |
| | | with open wound into | | | | |
| | | thorax | | | | |
| 860.4 | DMMPO | Traumatic | 0.06 | 241 | | 0 |
| | | pneumohemothorax w/o | | | | |
| | | open wound thorax | | | | |
| 860.5 | DMMPO | Traumatic | 0.30 | 250 | 1 | 0.5 |
| | | pneumohemothorax with | | | | |
| | | open wound thorax | | | | |
| 861.0 | DMMPO | Injury to heart w/o open | 0.98 | 229 | | 0 |
| | | wound into thorax | | | | |
| 861.10 | DMMPO | Unspec. injury of heart | 1.00 | 268 | 1 | 0.5 |
| | | w/open wound into thorax | | | | |
| 861.2 | DMMPO | Injury to lung, nos, closed | 0.30 | 250 | | 0 |
| 861.3 | DMMPO | Injury to lung nos, open | 0.30 | 250 | 1 | 0.5 |
| 863.0 | DMMPO | Stomach injury, w/o open | 1.00 | 390 | | 0 |
| | | wound into cavity | | | | |
| 864.10 | DMMPO | Unspecified injury to liver | 1.00 | 434 | 1 | 0.5 |
| | | with open wound into | | | | |
| | | cavity | | | | |
| 865 | DMMPO | Injury to spleen | 1.00 | 411 | | 0 |
| 866.0 | DMMPO | Injury kidney w/o open | 1.00 | 390 | | 0 |
| | | wound | | | | |
| 866.1 | DMMPO | Injury to kidney with open | 1.00 | 415 | 1 | 0.5 |
| | | wound into cavity | | | | |
| 867.0 | DMMPO | Injury to bladder urethra | 1.00 | 352 | | 0 |
| | | without open wound into | | | | |
| | | cavity | | | | |
| 867.1 | DMMPO | Injury to bladder and | 1.00 | 397 | 1 | 0.5 |
| | | urethrea with open wound | | | | |
| | | into cavity | | | | |
| 867.2 | DMMPO | Injury to ureter w/o open | 1.00 | 352 | | 0 |
| | | wound into cavity | | | | |
| 867.3 | DMMPO | Injury to ureter with open | 1.00 | 352 | 1 | 0.5 |
| | | wound into cavity | | | | |
| 867.4 | DMMPO | Injury to uterus w/o open | 1.00 | 352 | | 0 |
| | | wound into cavity | | | | |
| 867.5 | DMMPO | Injury to uterus with open | 1.00 | 352 | 1 | 0.5 |
| | | wound into cavity | | | | |
| 870 | DMMPO | Open wound of ocular | 0.63 | 30 | | 0 |
| | | adnexa | | | | |
| 870.3 | DMMPO | Penetrating wound of orbit | 0.63 | 30 | | 0 |
| | | without foreign body | | | | |
| 870.4 | DMMPO | Penetrating wound of orbit | 0.78 | 30 | | 0 |
| | | with foreign body | | | | |
| 871.5 | DMMPO | Penetration of eyeball with | 0.10 | 167 | | 0 |
| | | magnetic foreign body | | | | |
| 872 | DMMPO | Open wound of ear | 0.23 | 30 | 1 | 0.5 |
| 873.4 | DMMPO | Open wound of face | 0.22 | 226 | 1 | 0.5 |
| | | without mention of | | | | |
| | | complication | | | | |
| 873.8 | DMMPO | Open head wound w/o | 0.25 | 236 | 1 | 0.5 |
| | | complication | | | | |
| 873.9 | DMMPO | Open head wound with | 0.33 | 369 | 1 | 0.5 |
| | | complications | | | | |
| 874.8 | DMMPO | Open wound of other and | 0.25 | 236 | 1 | 0.5 |
| | | unspecified parts of neck | | | | |
| | | w/o complications | | | | |
| 875.0 | DMMPO | Open wound of chest | 0.33 | 266 | 2 | 0.5 |
| | | (wall) without | | | | |
| | | complication | | | | |
| 876.0 | DMMPO | Open wound of back | 0.40 | 278 | 1 | 0.5 |
| | | without complication | | | | |
| 877.0 | DMMPO | Open wound of buttock | 0.00 | | | 0 |
| | | without complication | | | | |
| 878 | DMMPO | Open wound of genital | 0.72 | 206 | 1 | 0.5 |
| | | organs (external) including | | | | |
| | | traumatic amputation | | | | |
| 879.2 | DMMPO | Open wound of abdominal | 0.50 | 397 | 2 | 0.5 |
| | | wall anterior w/o | | | | |
| | | complication | | | | |
| 879.6 | DMMPO | Open wound of other | 0.40 | 278 | 2 | 0.5 |
| | | unspecified parts of trunk | | | | |
| | | without complication | | | | |
| 879.8 | DMMPO | Open wound(s) (multiple) | 0.00 | | | 0 |
| | | of unspecified site(s) w/o | | | | |
| | | complication | | | | |
| 880 | DMMPO | Open wound of the | 0.25 | 228 | 1 | 0.5 |
| | | shoulder and upper arm | | | | |
| 881 | DMMPO | Open wound elbows, | 0.10 | 210 | 1 | 0.5 |
| | | forearm, and wrist | | | | |
| 882 | DMMPO | Open wound hand except | 0.00 | | | 0 |
| | | fingers alone | | | | |
| 883.0 | DMMPO | Open wound of fingers | 0.64 | 244 | 1 | 0.5 |
| | | without complication | | | | |
| 884.0 | DMMPO | Multiple/unspecified open | 0.64 | 244 | 1 | 0.5 |
| | | wound upper limb without | | | | |
| | | complication | | | | |
| 885 | DMMPO | Traumatic amputation of | 0.82 | 244 | 1 | 0.5 |
| | | thumb (complete) (partial) | | | | |
| 886 | DMMPO | Traumatic amputation of | 0.82 | 244 | 1 | 0.5 |
| | | other finger(s) (complete) | | | | |
| | | (partial) | | | | |
| 887 | DMMPO | Traumatic amputation of | 1.00 | 287 | 1 | 0.5 |
| | | arm and hand (complete) | | | | |
| | | (partial) | | | | |
| 890 | DMMPO | Open wound of hip and | 0.25 | 226 | 1 | 0.5 |
| | | thigh | | | | |
| 891 | DMMPO | Open wound of knee leg | 0.25 | 215 | 1 | 0.5 |
| | | (except thigh) and ankle | | | | |
| 892.0 | DMMPO | Open wound foot except | 0.64 | 244 | 1 | 0.5 |
| | | toes alone w/o | | | | |
| | | complication | | | | |
| 894.0 | DMMPO | Multiple/unspecified open | 0.54 | 60 | 1 | 0.5 |
| | | wound of lower limb w/o | | | | |
| | | complication | | | | |
| 895 | DMMPO | Traumatic amputation of | 1.00 | 244 | 1 | 0.5 |
| | | toe(s) (complete) (partial) | | | | |
| 896 | DMMPO | Traumatic amputation of | 1.00 | 297 | 1 | 0.5 |
| | | foot (complete) (partial) | | | | |
| 897 | DMMPO | Traumatic amputation of | 1.00 | 294 | 1 | 0.5 |
| | | leg(s) (complete) (partial) | | | | |
| 903 | DMMPO | Injury to blood vessels of | 1.00 | 198 | | 0 |
| | | upper extremity | | | | |
| 904 | DMMPO | Injury to blood vessels of | 1.00 | 200 | | 0 |
| | | lower extremity and | | | | |
| | | unspec. sites | | | | |
| 910.0 | DMMPO | Abrasion/friction burn of | 0.00 | | | 0 |
| | | face, neck, scalp w/o | | | | |
| | | infection | | | | |
| 916.0 | DMMPO | Abrasion/friction burn of | 0.00 | | | 0 |
| | | hip, thigh, leg, ankle w/o | | | | |
| | | infection | | | | |
| 916.1 | DMMPO | Abrasion/friction burn of | 0.00 | | | 0 |
| | | hip, thigh, leg, ankle with | | | | |
| | | infection | | | | |
| 916.2 | DMMPO | Blister hip & leg | 0.00 | | | 0 |
| 916.3 | DMMPO | Blister of hip thigh leg and | 0.00 | | | 0 |
| | | ankle infected | | | | |
| 916.4 | DMMPO | Insect bite nonvenom hip, | 0.00 | | | 0 |
| | | thigh, leg, ankle w/o | | | | |
| | | infection | | | | |
| 916.5 | DMMPO | Insect bite nonvenom hip, | 0.00 | | | 0 |
| | | thigh, leg, ankle, with | | | | |
| | | infection | | | | |
| 918.1 | DMMPO | Superficial injury cornea | 0.00 | | | 0 |
| 920 | DMMPO | Contusion of face scalp | 0.00 | | | 0 |
| | | and neck except eye(s) | | | | |
| 921.0 | DMMPO | Black eye | 0.00 | | | 0 |
| 922.1 | DMMPO | Contusion of chest wall | 0.00 | | | 0 |
| 922.2 | DMMPO | Contusion of abdominal | 0.00 | | | 0 |
| | | wall | | | | |
| 922.4 | DMMPO | Contusion of genital | 0.00 | | | 0 |
| | | organs | | | | |
| 924.1 | DMMPO | Contusion of knee and | 0.00 | | | 0 |
| | | lower leg | | | | |
| 924.2 | DMMPO | Contusion of ankle and | 0.00 | | | 0 |
| | | foot | | | | |
| 924.3 | DMMPO | Contusion of toe | 0.00 | | | 0 |
| 925 | DMMPO | Crushing injury of face, | 0.25 | 385 | 1 | 0.5 |
| | | scalp & neck | | | | |
| 926 | DMMPO | Crushing injury of trunk | 0.25 | 318 | 1 | 0.5 |
| 927 | DMMPO | crushing injury of upper | 0.61 | 317 | 1 | 0.5 |
| | | limb | | | | |
| 928 | DMMPO | Crushing injury of lower | 0.33 | 272 | 1 | 0.5 |
| | | limb | | | | |
| 930 | DMMPO | Foreign Body on External | 0.00 | | | 0 |
| | | Eye | | | | |
| 935 | DMMPO | Foreign body in mouth, | 1.00 | 200 | | 0 |
| | | esophagus and stomach | | | | |
| 941 | DMMPO | Burn of face, head, neck | 0.33 | 60 | | 0 |
| 942.0 | DMMPO | Burn of trunk, unspecified | 0.49 | 60 | | 0 |
| | | degree | | | | |
| 943.0 | DMMPO | Burn of upper limb except | 0.48 | 60 | | 0 |
| | | wrist and hand unspec. | | | | |
| | | degree | | | | |
| 944 | DMMPO | Burn of wrist and hand | 0.40 | 60 | | 0 |
| 945 | DMMPO | Burn of lower limb(s) | 0.50 | 120 | | 0 |
| 950 | DMMPO | Injury to optic nerve and | 0.60 | 120 | | 0 |
| | | pathways | | | | |
| 953.0 | DMMPO | Injury to cervical nerve | 0.35 | 60 | | 0 |
| | | root | | | | |
| 953.4 | DMMPO | Injury to brachial plexus | 0.57 | 60 | | 0 |
| 955.0 | DMMPO | Injury to axillary nerve | 0.64 | 60 | | 0 |
| 956.0 | DMMPO | Injury to sciatic nerve | 0.43 | 60 | | 0 |
| 959.01 | DMMPO | Other and unspecified | 0.35 | 60 | | 0 |
| | | injury to head | | | | |
| 959.09 | DMMPO | Other and unspecified | 0.35 | 60 | 1 | 0.5 |
| | | injury to face and neck | | | | |
| 959.7 | DMMPO | Other and unspecified | 0.14 | 60 | 1 | 0.5 |
| | | injury to knee leg ankle | | | | |
| | | and foot | | | | |
| 989.5 | DMMPO | Toxic effect of venom | 0.00 | | | 0 |
| 989.9 | DMMPO | Toxic effect unspec subst | 0.00 | | | 0 |
| | | chiefly | | | | |
| | | nonmedicinal/source | | | | |
| 991.3 | DMMPO | Frostbite | 0.00 | | | 0 |
| 991.6 | DMMPO | Hypothermia | 0.00 | | | 0 |
| 992.0 | DMMPO | Heat stroke and sun stroke | 0.00 | | | 0 |
| 992.2 | DMMPO | Heat cramps | 0.00 | | | 0 |
| 992.3 | DMMPO | Heat exhaustion | 0.00 | | | 0 |
| | | anhydrotic | | | | |
| 994.0 | DMMPO | Effects of lightning | 0.00 | | | 0 |
| 994.1 | DMMPO | Drowning and nonfatal | 0.00 | | | 0 |
| | | submersion | | | | |
| 994.2 | DMMPO | Effects of deprivation of | 0.00 | | | 0 |
| | | food | | | | |
| 994.3 | DMMPO | Effects of thirst | 0.00 | | | 0 |
| 994.4 | DMMPO | Exhaustion due to | 0.00 | | | 0 |
| | | exposure | | | | |
| 994.5 | DMMPO | Exhaustion due to | 0.00 | | | 0 |
| | | excessive exertion | | | | |
| 994.6 | DMMPO | Motion sickness | 0.00 | | | 0 |
| 994.8 | DMMPO | Electrocution and nonfatal | 0.00 | | | 0 |
| | | effects of electric current | | | | |
| 995.0 | DMMPO | Other anaphylactic shock | 0.00 | | | 0 |
| | | not elsewhere classified | | | | |
| E991.2 | DMMPO | Injury due to war ops from | 0.63 | 90 | 1 | 0.5 |
| | | other bullets (not | | | | |
| | | rubber/pellets) | | | | |
| E991.3 | DMMPO | Injury due to war ops from | 0.76 | 90 | 1 | 0.5 |
| | | antipersonnel bomb | | | | |
| | | fragment | | | | |
| E991.9 | DMMPO | Injury due to war ops other | 0.69 | 90 | 1 | 0.5 |
| | | unspecified fragments | | | | |
| E993 | DMMPO | Injury due to war ops by | 0.71 | 90 | 1 | 0.5 |
| | | other explosion | | | | |
| V01.5 | DMMPO | Contact with or exposure | 0.00 | | | 0 |
| | | to rabies | | | | |
| V79.0 | DMMPO | Screening for depression | 0.00 | | | 0 |
| 001.9 | Extended | Cholera unspecified | 0.00 | | | 0 |
| 002.0 | Extended | Typhoid fever | 0.00 | | | 0 |
| 004.9 | Extended | Shigellosis unspecified | 0.00 | | | 0 |
| 055.9 | Extended | Measles | 0.00 | | | 0 |
| 072.8 | Extended | Mumps with unspecified | 0.00 | | | 0 |
| | | complication | | | | |
| 072.9 | Extended | Mumps without | 0.00 | | | 0 |
| | | complication | | | | |
| 110.9 | Extended | Dermatophytosis, of | 0.00 | | | 0 |
| | | unspecified site | | | | |
| 128.9 | Extended | Other and unspecified | 0.00 | | | 0 |
| | | Helminthiasis | | | | |
| 132.9 | Extended | Pediculosis and Phthirus | 0.00 | | | 0 |
| | | Infestation | | | | |
| 133.0 | Extended | Scabies | 0.00 | | | 0 |
| 184.9 | Extended | Malignant neoplasm of | 0.00 | | | 0 |
| | | other and unspecified | | | | |
| | | female genital organs | | | | |
| 239.0 | Extended | Neoplasms of Unspecified | 0.80 | 60 | | 0 |
| | | Nature | | | | |
| 246.9 | Extended | Unspecified Disorder of | 0.00 | | | 0 |
| | | Thyroid | | | | |
| 250.00 | Extended | Diabetes Mellitus w/o | 0.00 | | | 0 |
| | | complication | | | | |
| 264.0 | Extended | Vitamin A deficiency | 0.00 | | | 0 |
| 269.8 | Extended | Other nutritional | 0.00 | | | 0 |
| | | deficiencies | | | | |
| 276.51 | Extended | Volume Depletion, | 0.00 | | | 0 |
| | | Dehydration | | | | |
| 277.89 | Extended | Other and unspecified | 0.00 | | | 0 |
| | | disorders of metabolism | | | | |
| 280.8 | Extended | Iron deficiency anemias | 0.00 | | | 0 |
| 300.00 | Extended | Anxiety states | 0.00 | | | 0 |
| 349.9 | Extended | Unspecified disorders of | 0.00 | | | 0 |
| | | nervous system | | | | |
| 366.00 | Extended | Cataract | 0.00 | | | 0 |
| 369.9 | Extended | Blindness and low vision | 0.00 | | | 0 |
| 372.30 | Extended | Conjunctivitis, unspecified | 0.00 | | | 0 |
| 379.90 | Extended | Other disorders of eye | 0.00 | | | 0 |
| 380.9 | Extended | Unspecified disorder of | 0.00 | | | 0 |
| | | external ear | | | | |
| 383.1 | Extended | Chronic mastoiditis | 0.00 | | | 0 |
| 386.10 | Extended | Other and unspecified | 0.00 | | | 0 |
| | | peripheral vertigo | | | | |
| 386.2 | Extended | Vertigo of central origin | 0.00 | | | 0 |
| 388.8 | Extended | Other disorders of ear | 0.07 | 30 | | 0 |
| 411.81 | Extended | Acute coronary occlusion | 0.00 | | | 0 |
| | | without myocardial | | | | |
| | | infarction | | | | |
| 428.40 | Extended | Heart failure | 0.00 | | | 0 |
| 437.9 | Extended | Cerebrovascular disease, | 0.00 | | | 0 |
| | | unspecified | | | | |
| 443.89 | Extended | Other peripheral vascular | 0.00 | | | 0 |
| | | disease | | | | |
| 459.9 | Extended | Unspecified circulatory | 0.00 | | | 0 |
| | | system disorder | | | | |
| 477.9 | Extended | Allergic rhinitis | 0.00 | | | 0 |
| 519.8 | Extended | Other diseases of | 0.06 | 30 | | 0 |
| | | respiratory system | | | | |
| 521.00 | Extended | Dental caries | 0.00 | | | 0 |
| 522.0 | Extended | Pulpitis | 0.00 | | | 0 |
| 525.19 | Extended | Other diseases and | 0.00 | | | 0 |
| | | conditions of the teeth and | | | | |
| | | supporting structures | | | | |
| 527.8 | Extended | Diseases of the salivary | 0.01 | 30 | | 0 |
| | | glands | | | | |
| 569.83 | Extended | Perforation of intestine | 0.58 | 30 | | 0 |
| 571.40 | Extended | Chronic hepatitis | 0.00 | | | 0 |
| 571.5 | Extended | Cirrhosis of liver without | 0.00 | | | 0 |
| | | alcohol | | | | |
| 594.9 | Extended | Calculus of lower urinary | 0.04 | 60 | | 0 |
| | | tract, unspecified | | | | |
| 599.8 | Extended | Urinary tract infection, site | 0.00 | | | 0 |
| | | not specified | | | | |
| 600.90 | Extended | Hyperplasia of prostate | 0.00 | | | 0 |
| 608.89 | Extended | Other disorders of male | 0.50 | 30 | | 0 |
| | | genital organs | | | | |
| 614.9 | Extended | Inflammatory disease of | 0.05 | 45 | | 0 |
| | | female pelvic | | | | |
| | | organs/tissues | | | | |
| 616.10 | Extended | Vaginitis and | 0.00 | | | 0 |
| | | vulvovaginitis | | | | |
| 623.5 | Extended | Leukorrhea not specified | 0.00 | | | 0 |
| | | as infective | | | | |
| 626.8 | Extended | Disorders of menstruation | 0.18 | 45 | | 0 |
| | | and other abnormal | | | | |
| | | bleeding from female | | | | |
| | | genital tract | | | | |
| 629.9 | Extended | Other disorders of female | 0.00 | | | 0 |
| | | genital organs | | | | |
| 650 | Extended | Normal delivery | 0.00 | | | 0 |
| 653.81 | Extended | Disproportion in | 0.00 | | | 0 |
| | | pregnancy labor and | | | | |
| | | delivery | | | | |
| 690.8 | Extended | Erythematosquamous | 0.00 | | | 0 |
| | | dermatosis | | | | |
| 691.8 | Extended | Atopic dermatitis and | 0.00 | | | 0 |
| | | related conditions | | | | |
| 692.9 | Extended | Contact Dermatitis, | 0.00 | | | 0 |
| | | unspecified cause | | | | |
| 693.8 | Extended | Dermatitis due to | 0.00 | | | 0 |
| | | substances taken internally | | | | |
| 696.1 | Extended | Other psoriasis and similar | 0.00 | | | 0 |
| | | disorders | | | | |
| 709.9 | Extended | Other disorders of skin and | 0.15 | 45 | | 0 |
| | | subcutaneous tissue | | | | |
| 714.0 | Extended | Rheumatoid arthritis | 0.00 | | | 0 |
| 733.90 | Extended | Disorder of bone and | 0.28 | 60 | | 0 |
| | | cartilage, unspecified | | | | |
| 779.9 | Extended | Other and ill-defined | 0.00 | | | 0 |
| | | conditions originating in | | | | |
| | | the perinatal period | | | | |
| 780.79 | Extended | Other malaise and fatigue | 0.00 | | | 0 |
| 780.96 | Extended | Generalized pain | 0.00 | | | 0 |
| 786.2 | Extended | Cough | 0.00 | | | 0 |
| 842.00 | Extended | Sprain of unspecified site | 0.00 | | | 0 |
| | | of wrist | | | | |

TABLE 90
EMRE Common Data: Bed Data

| | | | ORICULOS | ORWardLOS | NoORICULOS | NoORWardLOS |
|---|---|---|---|---|---|---|
| PC | Type | Description | (days) | (days) | (days) | (days) |
| 005 | DMMPO | Food poisoning bacterial | 0 | 0 | 0 | 5 |
| 006 | DMMPO | Amebiasis | 0 | 0 | 0 | 10 |
| 007.9 | DMMPO | Unspecified protozoal | 0 | 0 | 0 | 10 |
| | | intestinal disease | | | | |
| 008.45 | DMMPO | Intestinal infection due | 0 | 0 | 0 | 30 |
| | | to clostridium difficile | | | | |
| 008.8 | DMMPO | Intestinal infection due | 0 | 0 | 0 | 30 |
| | | to other organism not | | | | |
| | | classified | | | | |
| 010 | DMMPO | Primary tb | 0 | 0 | 0 | 180 |
| 037 | DMMPO | Tetanus | 0 | 0 | 0 | 14 |
| 038.9 | DMMPO | Unspecified septicemia | 0 | 0 | 1 | 13 |
| 042 | DMMPO | Human immunodeficiency | 0 | 0 | 0 | 180 |
| | | virus [HIV] disease | | | | |
| 047.9 | DMMPO | Viral meningitis | 0 | 0 | 1 | 13 |
| 052 | DMMPO | Varicella | 0 | 0 | 0 | 14 |
| 053 | DMMPO | Herpes zoster | 0 | 0 | 0 | 10 |
| 054.1 | DMMPO | Genital herpes | 0 | 0 | 0 | 3 |
| 057.0 | DMMPO | Fifth disease | 0 | 0 | 0 | 14 |
| 060 | DMMPO | Yellow fever | 0 | 0 | 1 | 180 |
| 061 | DMMPO | Dengue | 0 | 0 | 0 | 180 |
| 062 | DMMPO | Mosq. borne encephalitis | 0 | 0 | 1 | 13 |
| 063.9 | DMMPO | Tick borne encephalitis | 0 | 0 | 1 | 13 |
| 065 | DMMPO | Arthropod-borne hemorrhagic | 0 | 0 | 1 | 13 |
| | | fever | | | | |
| 066.40 | DMMPO | West nile fever, unspecified | 0 | 0 | 0 | 30 |
| 070.1 | DMMPO | Viral hepatitis | 0 | 0 | 0 | 30 |
| 071 | DMMPO | Rabies | 0 | 0 | 0 | 180 |
| 076 | DMMPO | Trachoma | 0 | 0 | 0 | 10 |
| 078.0 | DMMPO | Molluscom contagiosum | 0 | 0 | 0 | 1 |
| 078.1 | DMMPO | Viral warts | 0 | 0 | 0 | 1 |
| 078.4 | DMMPO | Hand, foot and mouth disease | 0 | 0 | 0 | 14 |
| 079.3 | DMMPO | Rhinovirus infection in conditions | 0 | 0 | 0 | 3 |
| | | elsewhere and of unspecified site | | | | |
| 079.99 | DMMPO | Unspecified viral infection | 0 | 0 | 0 | 180 |
| 082 | DMMPO | Tick-borne rickettsiosis | 0 | 0 | 0 | 10 |
| 084 | DMMPO | Malaria | 0 | 0 | 0 | 30 |
| 085 | DMMPO | Leishmaniasis, visceral | 0 | 0 | 0 | 30 |
| 086 | DMMPO | Trypanosomiasis | 0 | 0 | 0 | 14 |
| 091 | DMMPO | Early primary syphilis | 0 | 0 | 0 | 5 |
| 091.9 | DMMPO | Secondary syphilis, unspec | 0 | 0 | 0 | 5 |
| 094 | DMMPO | Neurosyphilis | 0 | 0 | 1 | 180 |
| 098.5 | DMMPO | Gonococcal arthritis | 0 | 0 | 0 | 14 |
| 099.4 | DMMPO | Nongonnococcal urethritis | 0 | 0 | 0 | 1 |
| 100 | DMMPO | Leptospirosis | 0 | 0 | 2 | 12 |
| 274 | DMMPO | Gout | 0 | 0 | 0 | 5 |
| 276 | DMMPO | Disorder of fluid, electrolyte + | 0 | 0 | 0 | 3 |
| | | acid base balance | | | | |
| 296.0 | DMMPO | Bipolar disorder, single manic | 0 | 0 | 0 | 30 |
| | | episode | | | | |
| 298.9 | DMMPO | Unspecified psychosis | 0 | 0 | 0 | 30 |
| 309.0 | DMMPO | Adjustment disorder with depressed | 0 | 0 | 0 | 30 |
| | | mood | | | | |
| 309.81 | DMMPO | Ptsd | 0 | 0 | 0 | 30 |
| 309.9 | DMMPO | Unspecified adjustment reaction | 0 | 0 | 0 | 14 |
| 310.2 | DMMPO | Post concussion syndrome | 0 | 0 | 0 | 7 |
| 345.2 | DMMPO | Epilepsy petit mal | 0 | 0 | 1 | 180 |
| 345.3 | DMMPO | Epilepsy grand mal | 0 | 0 | 1 | 180 |
| 346 | DMMPO | Migraine | 0 | 0 | 0 | 3 |
| 361 | DMMPO | Retinal detachment | 0 | 0 | 0 | 7 |
| 364.3 | DMMPO | Uveitis nos | 0 | 0 | 0 | 7 |
| 365 | DMMPO | Glaucoma | 0 | 0 | 0 | 180 |
| 370.0 | DMMPO | Corneal ulcer | 0 | 0 | 0 | 5 |
| 379.31 | DMMPO | Aphakia | 0 | 0 | 0 | 7 |
| 380.1 | DMMPO | Infective otitis externa | 0 | 0 | 0 | 1 |
| 380.4 | DMMPO | Impacted cerumen | 0 | 0 | 0 | 3 |
| 381 | DMMPO | Acute nonsuppurative otitis | 0 | 0 | 0 | 3 |
| | | media | | | | |
| 381.9 | DMMPO | Unspecified eustachian tube | 0 | 0 | 0 | 3 |
| | | disorder | | | | |
| 384.2 | DMMPO | Perforated tympanic membrane | 0 | 0 | 0 | 10 |
| 388.3 | DMMPO | Tinnitus, unspecified | 0 | 0 | 0 | 3 |
| 389.9 | DMMPO | Unspecified hearing loss | 0 | 0 | 0 | 5 |
| 401 | DMMPO | Essential hypertension | 0 | 0 | 0 | 14 |
| 410 | DMMPO | Myocardial infarction | 0 | 0 | 1 | 180 |
| 413.9 | DMMPO | Other and unspecified angina | 0 | 0 | 0 | 180 |
| | | pectoris | | | | |
| 427.9 | DMMPO | Cardiac dysryhthmia unspecified | 0 | 0 | 0 | 180 |
| 453.4 | DMMPO | Venous embolism/thrombus of | 0 | 0 | 1 | 30 |
| | | deep vessels lower extremity | | | | |
| 462 | DMMPO | Acute pharyngitis | 0 | 0 | 0 | 7 |
| 465 | DMMPO | Acute uri of multiple or | 0 | 0 | 0 | 5 |
| | | unspecified sites | | | | |
| 466 | DMMPO | Acute bronchitis & bronchiolitis | 0 | 0 | 0 | 10 |
| 475 | DMMPO | Peritonsillar abscess | 0 | 10 | 0 | 10 |
| 486 | DMMPO | Pneumonia, organism unspecified | 0 | 0 | 0 | 7 |
| 491 | DMMPO | Chronic bronchitis | 0 | 0 | 0 | 14 |
| 492 | DMMPO | Emphysema | 0 | 0 | 0 | 14 |
| 493.9 | DMMPO | Asthma | 0 | 0 | 0 | 1 |
| 523 | DMMPO | Gingival and periodontal | 0 | 0 | 0 | 2 |
| | | disease | | | | |
| 530.2 | DMMPO | Ulcer of esophagus | 0 | 0 | 0 | 14 |
| 530.81 | DMMPO | Gastroesophageal reflux | 0 | 0 | 0 | 5 |
| 531 | DMMPO | Gastric ulcer | 0 | 0 | 0 | 14 |
| 532 | DMMPO | Duodenal ulcer | 0 | 5 | 0 | 5 |
| 540.9 | DMMPO | Acute appendicitis without | 0 | 30 | 0 | 30 |
| | | mention of peritonitis | | | | |
| 541 | DMMPO | Appendicitis, unspecified | 0 | 30 | 0 | 30 |
| 550.9 | DMMPO | Unilateral inguinal hernia | 0 | 30 | 0 | 30 |
| 553.1 | DMMPO | Umbilical hernia | 0 | 14 | 0 | 14 |
| 553.9 | DMMPO | Hernia nos | 0 | 14 | 0 | 14 |
| 564.0 | DMMPO | Constipation | 0 | 0 | 0 | 1 |
| 564.1 | DMMPO | Irritable bowel disease | 0 | 0 | 0 | 30 |
| 566 | DMMPO | Abscess of anal and rectal | 0 | 30 | 0 | 30 |
| | | regions | | | | |
| 567.9 | DMMPO | Unspecified peritonitis | 0 | 0 | 0 | 30 |
| 574 | DMMPO | Cholelithiasis | 0 | 14 | 0 | 14 |
| 577.0 | DMMPO | Acute pancreatitis | 0 | 0 | 1 | 180 |
| 577.1 | DMMPO | Chronic pancreatitis | 0 | 0 | 1 | 180 |
| 578.9 | DMMPO | Hemorrhage of gastrointestinal | 0 | 0 | 0 | 7 |
| | | tract unspecified | | | | |
| 584.9 | DMMPO | Acute renal failure unspecified | 0 | 0 | 2 | 180 |
| 592 | DMMPO | Calculus of kidney | 0 | 0 | 0 | 7 |
| 599.0 | DMMPO | Unspecified urinary tract | 0 | 0 | 0 | 3 |
| | | infection | | | | |
| 599.7 | DMMPO | Hematuria | 0 | 0 | 0 | 3 |
| 608.2 | DMMPO | Torsion of testes | 0 | 180 | 0 | 180 |
| 608.4 | DMMPO | Other inflammatory disorders | 0 | 0 | 0 | 10 |
| | | of male genital organs | | | | |
| 611.7 | DMMPO | Breast lump | 0 | 0 | 0 | 14 |
| 633 | DMMPO | Ectopic preg | 0 | 30 | 0 | 30 |
| 634 | DMMPO | Spontaneous abortion | 0 | 30 | 0 | 30 |
| 681 | DMMPO | Cellulitis and abscess of | 0 | 0 | 0 | 7 |
| | | finger and toe | | | | |
| 682.0 | DMMPO | Cellulitis and abscess of | 0 | 0 | 0 | 7 |
| | | face | | | | |
| 682.6 | DMMPO | Cellulitis and abscess of | 0 | 0 | 0 | 7 |
| | | leg except foot | | | | |
| 682.7 | DMMPO | Cellulitis and abscess of | 0 | 0 | 0 | 7 |
| | | foot except toes | | | | |
| 682.9 | DMMPO | Cellulitis and abscess of | 0 | 0 | 0 | 7 |
| | | unspecified parts | | | | |
| 719.41 | DMMPO | Pain in joint shoulder | 0 | 0 | 0 | 14 |
| 719.46 | DMMPO | Pain in joint lower leg | 0 | 0 | 0 | 14 |
| 719.47 | DMMPO | Pain in joint ankle/foot | 0 | 0 | 0 | 14 |
| 722.1 | DMMPO | Displacement lumbar | 0 | 0 | 0 | 30 |
| | | intervertebral disc w/o | | | | |
| | | myelopathy | | | | |
| 723.0 | DMMPO | Spinal stenosis in cervical | 0 | 0 | 0 | 30 |
| | | region | | | | |
| 724.02 | DMMPO | Spinal stenosis of lumbar | 0 | 0 | 0 | 30 |
| | | region | | | | |
| 724.2 | DMMPO | Lumbago | 0 | 0 | 0 | 5 |
| 724.3 | DMMPO | Sciatica | 0 | 0 | 0 | 30 |
| 724.4 | DMMPO | Lumbar sprain (thoracic/ | 0 | 0 | 0 | 5 |
| | | lumbosacral) neuritis or | | | | |
| | | radiculitis, unspec | | | | |
| 724.5 | DMMPO | Backache unspecified | 0 | 0 | 0 | 5 |
| 726.10 | DMMPO | Disorders of bursae and | 0 | 0 | 0 | 14 |
| | | tendons in shoulder | | | | |
| | | unspecified | | | | |
| 726.12 | DMMPO | Bicipital tenosynovitis | 0 | 0 | 0 | 14 |
| 726.3 | DMMPO | Enthesopathy of elbow region | 0 | 0 | 0 | 14 |
| 726.4 | DMMPO | Enthesopathy of wrist and carpus | 0 | 0 | 0 | 14 |
| 726.5 | DMMPO | Enthesopathy of hip region | 0 | 0 | 0 | 14 |
| 726.6 | DMMPO | Enthesopathy of knee | 0 | 0 | 0 | 14 |
| 726.7 | DMMPO | Enthesopathy of ankle and tarsus | 0 | 0 | 0 | 14 |
| 729.0 | DMMPO | Rheumatism unspecified and | 0 | 0 | 0 | 14 |
| | | fibrositis | | | | |
| 729.5 | DMMPO | Pain in limb | 0 | 0 | 0 | 14 |
| 780.0 | DMMPO | Alterations of consciousness | 0 | 0 | 0 | 10 |
| 780.2 | DMMPO | Syncope | 0 | 0 | 0 | 3 |
| 780.39 | DMMPO | Other convulsions | 0 | 0 | 0 | 10 |
| 780.5 | DMMPO | Sleep disturbances | 0 | 0 | 0 | 4 |
| 780.6 | DMMPO | Fever | 0 | 0 | 0 | 5 |
| 782.1 | DMMPO | Rash and other nonspecific | 0 | 0 | 0 | 4 |
| | | skin eruptions | | | | |
| 782.3 | DMMPO | Edema | 0 | 0 | 0 | 4 |
| 783.0 | DMMPO | Anorexia | 0 | 0 | 0 | 4 |
| 784.0 | DMMPO | Headache | 0 | 0 | 0 | 10 |
| 784.7 | DMMPO | Epistaxis | 0 | 0 | 0 | 4 |
| 784.8 | DMMPO | Hemorrhage from throat | 0 | 0 | 0 | 10 |
| 786.5 | DMMPO | Chest pain | 0 | 0 | 0 | 10 |
| 787.0 | DMMPO | Nausea and vomiting | 0 | 0 | 0 | 4 |
| 787.91 | DMMPO | Diarrhea nos | 0 | 0 | 0 | 5 |
| 789.00 | DMMPO | Abdominal pain unspecified | 0 | 0 | 0 | 10 |
| | | site | | | | |
| 800.0 | DMMPO | Closed fracture of vault of | 0 | 0 | 2 | 180 |
| | | skull without intracranial | | | | |
| | | injury | | | | |
| 801.0 | DMMPO | Closed fracture of base of | 2 | 180 | 2 | 180 |
| | | skull without intracranial | | | | |
| | | injury | | | | |
| 801.76 | DMMPO | Open fracture base of | 3 | 180 | 3 | 180 |
| | | skull with subarachnoid, | | | | |
| | | subdural and extradural | | | | |
| | | hemorrhage with loss of | | | | |
| | | consciousness of | | | | |
| | | unspecified duration | | | | |
| 802.0 | DMMPO | Closed fracture of nasal bones | 0 | 180 | 0 | 180 |
| 802.1 | DMMPO | Open fracture of nasal bones | 0 | 180 | 0 | 180 |
| 802.6 | DMMPO | Fracture orbital floor closed | 0 | 180 | 0 | 180 |
| | | (blowout) | | | | |
| 802.7 | DMMPO | Fracture orbital floor open | 0 | 180 | 0 | 180 |
| | | (blowout) | | | | |
| 802.8 | DMMPO | Closed fracture of other facial | 0 | 180 | 0 | 180 |
| | | bones | | | | |
| 802.9 | DMMPO | Open fracture of other facial | 0 | 180 | 0 | 180 |
| | | bones | | | | |
| 805 | DMMPO | Closed fracture of cervical | 2 | 180 | 2 | 180 |
| | | vertebra w/o spinal cord injury | | | | |
| 806.1 | DMMPO | Open fracture of cervical vertebra | 2 | 180 | 2 | 180 |
| | | with spinal cord injury | | | | |
| 806.2 | DMMPO | Closed fracture of dorsal vertebra | 2 | 180 | 2 | 180 |
| | | with spinal cord injury | | | | |
| 806.3 | DMMPO | Open fracture of dorsal vertebra | 2 | 180 | 2 | 180 |
| | | with spinal cord injury | | | | |
| 806.4 | DMMPO | Closed fracture of lumbar spine | 2 | 180 | 2 | 180 |
| | | with spinal cord injury | | | | |
| 806.5 | DMMPO | Open fracture of lumbar spine | 2 | 180 | 2 | 180 |
| | | with spinal cord injury | | | | |
| 806.60 | DMMPO | Closed fracture sacrum and coccyx | 2 | 180 | 2 | 180 |
| | | w/unspec. spinal cord injury | | | | |
| 806.70 | DMMPO | Open fracture sacrum and coccyx | 2 | 180 | 2 | 180 |
| | | w/unspec. spinal cord injury | | | | |
| 807.0 | DMMPO | Closed fracture of rib(s) | 0 | 30 | 0 | 30 |
| 807.1 | DMMPO | Open fracture of rib(s) | 0 | 180 | 0 | 180 |
| 807.2 | DMMPO | Closed fracture of sternum | 0 | 180 | 0 | 180 |
| 807.3 | DMMPO | Open fracture of sternum | 0 | 180 | 0 | 180 |
| 808.8 | DMMPO | Fracture of pelvis unspecified, | 1 | 180 | 1 | 180 |
| | | closed | | | | |
| 808.9 | DMMPO | Fracture of pelvis unspecified, | 1 | 180 | 1 | 180 |
| | | open | | | | |
| 810.0 | DMMPO | Clavicle fracture, closed | 0 | 30 | 0 | 30 |
| 810.1 | DMMPO | Clavicle fracture, open | 0 | 180 | 0 | 180 |
| 810.12 | DMMPO | Open fracture of shaft of clavicle | 0 | 180 | 0 | 180 |
| 811.0 | DMMPO | Fracture of scapula, closed | 0 | 180 | 0 | 180 |
| 811.1 | DMMPO | Fracture of scapula, open | 0 | 180 | 0 | 180 |
| 812.00 | DMMPO | Fracture of unspecified part | 0 | 180 | 0 | 180 |
| | | of upper end of humerus, closed | | | | |
| 813.8 | DMMPO | Fracture unspecified part of | 0 | 180 | 0 | 180 |
| | | radius and ulna closed | | | | |
| 813.9 | DMMPO | Fracture unspecified part of | 0 | 180 | 0 | 180 |
| | | radius and ulna open | | | | |
| 815.0 | DMMPO | Closed fracture of metacarpal | 0 | 180 | 0 | 180 |
| | | bones | | | | |
| 816.0 | DMMPO | Phalanges fracture, closed | 0 | 180 | 0 | 180 |
| 816.1 | DMMPO | Phalanges fracture, open | 0 | 30 | 0 | 30 |
| 817.0 | DMMPO | Multiple closed fractures of | 0 | 30 | 0 | 30 |
| | | hand bones | | | | |
| 817.1 | DMMPO | Multiple open fracture of | 0 | 180 | 0 | 180 |
| | | hand bones | | | | |
| 820.8 | DMMPO | Fracture of femur neck, closed | 0 | 180 | 0 | 180 |
| 820.9 | DMMPO | Fracture of femur neck, open | 0 | 180 | 0 | 180 |
| 821.01 | DMMPO | Fracture shaft femur, closed | 0 | 180 | 0 | 180 |
| 821.11 | DMMPO | Fracture shaft of femur, open | 0 | 180 | 0 | 180 |
| 822.0 | DMMPO | Closed fracture of patella | 0 | 180 | 0 | 180 |
| 822.1 | DMMPO | Open fracture of patella | 0 | 180 | 0 | 180 |
| 823.82 | DMMPO | Fracture tib fib, closed | 0 | 180 | 0 | 180 |
| 823.9 | DMMPO | Fracture of unspecified part of | 0 | 180 | 0 | 180 |
| | | tibia and fibula open | | | | |
| 824.8 | DMMPO | Fracture ankle, nos, closed | 0 | 180 | 0 | 180 |
| 824.9 | DMMPO | Ankle fracture, open | 0 | 180 | 0 | 180 |
| 825.0 | DMMPO | Fracture to calcaneus, closed | 0 | 180 | 0 | 180 |
| 826.0 | DMMPO | Closed fracture of one or more | 0 | 180 | 0 | 180 |
| | | phalanges of foot | | | | |
| 829.0 | DMMPO | Fracture of unspecified bone, | 0 | 180 | 0 | 180 |
| | | closed | | | | |
| 830.0 | DMMPO | Closed dislocation of jaw | 0 | 0 | 0 | 14 |
| 830.1 | DMMPO | Open dislocation of jaw | 0 | 180 | 0 | 180 |
| 831 | DMMPO | Dislocation shoulder | 0 | 0 | 0 | 4 |
| 831.04 | DMMPO | Closed dislocation of | 0 | 0 | 0 | 14 |
| | | acromioclavicular joint | | | | |
| 831.1 | DMMPO | Dislocation of shoulder, open | 0 | 180 | 0 | 180 |
| 832.0 | DMMPO | Dislocation elbow, closed | 0 | 0 | 0 | 30 |
| 832.1 | DMMPO | Dislocation elbow, open | 0 | 180 | 0 | 180 |
| 833 | DMMPO | Dislocation wrist closed | 0 | 30 | 0 | 30 |
| 833.1 | DMMPO | Dislocated wrist, open | 0 | 30 | 0 | 30 |
| 834.0 | DMMPO | Dislocation of finger, closed | 0 | 0 | 0 | 3 |
| 834.1 | DMMPO | Dislocation of finger, open | 0 | 30 | 0 | 30 |
| 835 | DMMPO | Closed dislocation of hip | 0 | 0 | 0 | 30 |
| 835.1 | DMMPO | Hip dislocation open | 0 | 180 | 0 | 180 |
| 836.0 | DMMPO | Medial meniscus tear | 0 | 0 | 0 | 2 |
| 836.1 | DMMPO | Lateral meniscus tear | 0 | 0 | 0 | 2 |
| 836.2 | DMMPO | Meniscus tear of knee | 0 | 0 | 0 | 2 |
| 836.5 | DMMPO | Dislocation knee, closed | 0 | 0 | 0 | 14 |
| 836.6 | DMMPO | Other dislocation of knee open | 0 | 180 | 0 | 180 |
| 839.01 | DMMPO | Closed dislocation first | 0 | 0 | 1 | 13 |
| | | cervical vertebra | | | | |
| 840.4 | DMMPO | Rotator cuff sprain | 0 | 0 | 0 | 3 |
| 840.9 | DMMPO | Sprain shoulder | 0 | 0 | 0 | 3 |
| 843 | DMMPO | Sprains and strains of hip | 0 | 0 | 0 | 3 |
| | | and thigh | | | | |
| 844.9 | DMMPO | Sprain, knee | 0 | 0 | 0 | 5 |
| 845 | DMMPO | Sprain of ankle | 0 | 0 | 0 | 5 |
| 846 | DMMPO | Sprains and strains of socroiliac | 0 | 0 | 0 | 5 |
| | | region | | | | |
| 846.0 | DMMPO | Sprain of lumbosacral (joint) | 0 | 0 | 0 | 5 |
| | | (ligament) | | | | |
| 847.2 | DMMPO | Sprain lumbar region | 0 | 0 | 0 | 3 |
| 847.3 | DMMPO | Sprain of sacrum | 0 | 0 | 0 | 3 |
| 848.1 | DMMPO | Jaw sprain | 0 | 0 | 0 | 3 |
| 848.3 | DMMPO | Sprain of ribs | 0 | 0 | 0 | 3 |
| 850.9 | DMMPO | Concussion | 0 | 0 | 0 | 7 |
| 851.0 | DMMPO | Cortex (Cerebral) contusion w/o open | 0 | 0 | 2 | 30 |
| | | intracranial wound | | | | |
| 851.01 | DMMPO | Cortex (Cerebral) contusion w/o open | 0 | 0 | 2 | 30 |
| | | wound no loss of consciousness | | | | |
| 852 | DMMPO | Subarachnoid subdural extradural | 2 | 180 | 2 | 180 |
| | | hemorrhage injury | | | | |
| 853 | DMMPO | Other and unspecified intracranial | 2 | 30 | 2 | 30 |
| | | hemorrhage injury w/o open wound | | | | |
| 853.15 | DMMPO | Unspecified intracranial hemorrhage | 3 | 180 | 3 | 180 |
| | | with open intracranial wound | | | | |
| 860.0 | DMMPO | Traumatic pneumothorax w/o open | 0 | 180 | 0 | 180 |
| | | wound into thorax | | | | |
| 860.1 | DMMPO | Traumatic pneumothorax w/open | 2 | 180 | 2 | 180 |
| | | wound into thorax | | | | |
| 860.2 | DMMPO | Traumatic hemothorax w/o open | 2 | 180 | 2 | 180 |
| | | wound into thorax | | | | |
| 860.3 | DMMPO | Traumatic hemothorax with open | 2 | 180 | 2 | 180 |
| | | wound into thorax | | | | |
| 860.4 | DMMPO | Traumatic pneumohemothorax w/o | 2 | 180 | 2 | 180 |
| | | open wound thorax | | | | |
| 860.5 | DMMPO | Traumatic pneumohemothorax with | 2 | 180 | 2 | 180 |
| | | open wound thorax | | | | |
| 861.0 | DMMPO | Injury to heart w/o open wound | 3 | 180 | 2 | 180 |
| | | into thorax | | | | |
| 861.10 | DMMPO | Unspec. injury of heart | 3 | 180 | 3 | 180 |
| | | w/open wound into thorax | | | | |
| 861.2 | DMMPO | Injury to lung, nos, closed | 2 | 180 | 2 | 180 |
| 861.3 | DMMPO | Injury to lung nos, open | 2 | 180 | 2 | 180 |
| 863.0 | DMMPO | Stomach injury, w/o | 0 | 180 | 0 | 180 |
| | | open wound into cavity | | | | |
| 864.10 | DMMPO | Unspecified injury to liver | 1 | 180 | 1 | 180 |
| | | with open wound into cavity | | | | |
| 865 | DMMPO | Injury to spleen | 1 | 180 | 1 | 180 |
| 866.0 | DMMPO | Injury kidney w/o open wound | 0 | 180 | 0 | 180 |
| 866.1 | DMMPO | Injury to kidney with | 0 | 180 | 0 | 180 |
| | | open wound into cavity | | | | |
| 867.0 | DMMPO | Injury to bladder urethra | 0 | 180 | 0 | 180 |
| | | without open wound into cavity | | | | |
| 867.1 | DMMPO | Injury to bladder and urethrea | 0 | 180 | 0 | 180 |
| | | with open wound into cavity | | | | |
| 867.2 | DMMPO | Injury to ureter w/o open | 0 | 180 | 0 | 180 |
| | | wound into cavity | | | | |
| 867.3 | DMMPO | Injury to ureter with open | 0 | 180 | 0 | 180 |
| | | wound into cavity | | | | |
| 867.4 | DMMPO | Injury to uterus w/o open | 0 | 180 | 0 | 180 |
| | | wound into cavity | | | | |
| 867.5 | DMMPO | Injury to uterus with open | 0 | 180 | 0 | 180 |
| | | wound into cavity | | | | |
| 870 | DMMPO | Open wound of ocular adnexa | 0 | 7 | 0 | 7 |
| 870.3 | DMMPO | Penetrating wound of orbit | 0 | 7 | 0 | 7 |
| | | without foreign body | | | | |
| 870.4 | DMMPO | Penetrating wound of orbit | 0 | 7 | 0 | 7 |
| | | with foreign body | | | | |
| 871.5 | DMMPO | Penetration of eyeball with | 0 | 30 | 0 | 30 |
| | | magnetic foreign body | | | | |
| 872 | DMMPO | Open wound of ear | 0 | 3 | 0 | 3 |
| 873.4 | DMMPO | Open wound of face without | 0 | 5 | 0 | 5 |
| | | mention of complication | | | | |
| 873.8 | DMMPO | Open head wound w/o | 0 | 5 | 0 | 5 |
| | | complication | | | | |
| 873.9 | DMMPO | Open head wound with | 1 | 13 | 1 | 13 |
| | | complications | | | | |
| 874.8 | DMMPO | Open wound of other | 0 | 5 | 0 | 5 |
| | | and unspecified parts of | | | | |
| | | neck w/o complications | | | | |
| 875.0 | DMMPO | Open wound of chest (wall) | 0 | 5 | 0 | 5 |
| | | without complication | | | | |
| 876.0 | DMMPO | Open wound of back without | 0 | 14 | 0 | 14 |
| | | complication | | | | |
| 877.0 | DMMPO | Open wound of buttock without | 0 | 0 | 0 | 3 |
| | | complication | | | | |
| 878 | DMMPO | Open wound of genital organs | 0 | 30 | 0 | 30 |
| | | (external) including traumatic | | | | |
| | | amputation | | | | |
| 879.2 | DMMPO | Open wound of abdominal wall | 0 | 5 | 0 | 5 |
| | | anterior w/o complication | | | | |
| 879.6 | DMMPO | Open wound of other | 0 | 14 | 0 | 14 |
| | | unspecified parts of trunk | | | | |
| | | without complication | | | | |
| 879.8 | DMMPO | Open wound(s) (multiple) | 0 | 0 | 0 | 14 |
| | | of unspecified site(s) w/o | | | | |
| | | complication | | | | |
| 880 | DMMPO | Open wound of the shoulder | 0 | 3 | 0 | 3 |
| | | and upper arm | | | | |
| 881 | DMMPO | Open wound elbows, forearm, | 0 | 3 | 0 | 3 |
| | | and wrist | | | | |
| 882 | DMMPO | Open wound hand except | 0 | 0 | 0 | 180 |
| | | fingers alone | | | | |
| 883.0 | DMMPO | Open wound of fingers without | 0 | 14 | 0 | 14 |
| | | complication | | | | |
| 884.0 | DMMPO | Multiple/unspecified open | 0 | 180 | 0 | 180 |
| | | wound upper limb without | | | | |
| | | complication | | | | |
| 885 | DMMPO | Traumatic amputation of | 0 | 14 | 0 | 14 |
| | | thumb (complete) (partial) | | | | |
| 886 | DMMPO | Traumatic amputation of other | 0 | 180 | 0 | 180 |
| | | finger(s) (complete) (partial) | | | | |
| 887 | DMMPO | Traumatic amputation of arm and | 0 | 180 | 0 | 180 |
| | | hand (complete) (partial) | | | | |
| 890 | DMMPO | Open wound of hip and thigh | 0 | 7 | 0 | 7 |
| 891 | DMMPO | Open wound of knee leg (except | 0 | 7 | 0 | 7 |
| | | thigh) and ankle | | | | |
| 892.0 | DMMPO | Open wound foot except toes | 0 | 14 | 0 | 14 |
| | | alone w/o complication | | | | |
| 894.0 | DMMPO | Multiple/unspecified open wound | 0 | 5 | 0 | 5 |
| | | of lower limb w/o complication | | | | |
| 895 | DMMPO | Traumatic amputation of toe(s) | 0 | 180 | 0 | 180 |
| | | (complete) (partial) | | | | |
| 896 | DMMPO | Traumatic amputation of foot | 0 | 180 | 0 | 180 |
| | | (complete) (partial) | | | | |
| 897 | DMMPO | Traumatic amputation of leg(s) | 2 | 180 | 2 | 180 |
| | | (complete) (partial) | | | | |
| 903 | DMMPO | Injury to blood vessels | 0 | 180 | 0 | 180 |
| | | of upper extremity | | | | |
| 904 | DMMPO | Injury to blood vessels | 1 | 180 | 1 | 180 |
| | | of lower extremity and | | | | |
| | | unspec. sites | | | | |
| 910.0 | DMMPO | Abrasion/friction burn | 0 | 0 | 0 | 3 |
| | | of face, neck, scalp w/o | | | | |
| | | infection | | | | |
| 916.0 | DMMPO | Abrasion/friction burn | 0 | 0 | 0 | 3 |
| | | of hip, thigh, leg, ankle | | | | |
| | | w/o infection | | | | |
| 916.1 | DMMPO | Abrasion/friction burn | 0 | 0 | 0 | 10 |
| | | of hip, thigh, leg, ankle | | | | |
| | | with infection | | | | |
| 916.2 | DMMPO | Blister hip & leg | 0 | 0 | 0 | 3 |
| 916.3 | DMMPO | Blister of hip thigh leg | 0 | 0 | 0 | 10 |
| | | and ankle infected | | | | |
| 916.4 | DMMPO | Insect bite nonvenom hip, | 0 | 0 | 0 | 3 |
| | | thigh, leg, ankle w/o | | | | |
| | | infection | | | | |
| 916.5 | DMMPO | Insect bite nonvenom hip, | 0 | 0 | 0 | 10 |
| | | thigh, leg, ankle, with | | | | |
| | | infection | | | | |
| 918.1 | DMMPO | Superficial injury cornea | 0 | 0 | 0 | 3 |
| 920 | DMMPO | Contusion of face scalp | 0 | 0 | 0 | 2 |
| | | and neck except eye(s) | | | | |
| 921.0 | DMMPO | Black eye | 0 | 0 | 0 | 2 |
| 922.1 | DMMPO | Contusion of chest wall | 0 | 0 | 0 | 2 |
| 922.2 | DMMPO | Contusion of abdominal | 0 | 0 | 0 | 2 |
| | | wall | | | | |
| 922.4 | DMMPO | Contusion of genital organs | 0 | 0 | 0 | 3 |
| 924.1 | DMMPO | Contusion of knee and | 0 | 0 | 0 | 2 |
| | | lower leg | | | | |
| 924.2 | DMMPO | Contusion of ankle and foot | 0 | 0 | 0 | 2 |
| 924.3 | DMMPO | Contusion of toe | 0 | 0 | 0 | 2 |
| 925 | DMMPO | Crushing injury of face, | 1 | 180 | 1 | 180 |
| | | scalp & neck | | | | |
| 926 | DMMPO | Crushing injury of trunk | 2 | 180 | 2 | 180 |
| 927 | DMMPO | crushing injury of upper limb | 1 | 180 | 1 | 180 |
| 928 | DMMPO | Crushing injury of lower limb | 1 | 180 | 1 | 180 |
| 930 | DMMPO | Foreign Body on External Eye | 0 | 0 | 0 | 3 |
| 935 | DMMPO | Foreign body in mouth, | 0 | 7 | 0 | 7 |
| | | esophagus and stomach | | | | |
| 941 | DMMPO | Burn of face, head, neck | 2 | 3 | 2 | 3 |
| 942.0 | DMMPO | Burn of trunk, unspecified | 2 | 30 | 2 | 30 |
| | | degree | | | | |
| 943.0 | DMMPO | Burn of upper limb except | 1 | 13 | 1 | 13 |
| | | wrist and hand unspec. degree | | | | |
| 944 | DMMPO | Burn of wrist and hand | 0 | 14 | 0 | 14 |
| 945 | DMMPO | Burn of lower limb(s) | 1 | 13 | 1 | 13 |
| 950 | DMMPO | Injury to optic nerve and | 0 | 30 | 0 | 30 |
| | | pathways | | | | |
| 953.0 | DMMPO | Injury to cervical nerve root | 0 | 10 | 0 | 10 |
| 953.4 | DMMPO | Injury to brachial plexus | 0 | 30 | 0 | 30 |
| 955.0 | DMMPO | Injury to axillary nerve | 0 | 30 | 0 | 30 |
| 956.0 | DMMPO | Injury to sciatic nerve | 0 | 30 | 0 | 30 |
| 959.01 | DMMPO | Other and unspecified injury | 0 | 14 | 0 | 14 |
| | | to head | | | | |
| 959.09 | DMMPO | Other and unspecified | 0 | 14 | 0 | 14 |
| | | injury to face and neck | | | | |
| 959.7 | DMMPO | Other and unspecified | 0 | 14 | 0 | 14 |
| | | injury to knee leg ankle | | | | |
| | | and foot | | | | |
| 989.5 | DMMPO | Toxic effect of venom | 0 | 0 | 0 | 3 |
| 989.9 | DMMPO | Toxic effect unspec subst | 0 | 0 | 0 | 7 |
| | | chiefly nonmedicinal/source | | | | |
| 991.3 | DMMPO | Frostbite | 0 | 0 | 0 | 5 |
| 991.6 | DMMPO | Hypothermia | 0 | 0 | 1 | 9 |
| 992.0 | DMMPO | Heat stroke and sun stroke | 0 | 0 | 0 | 180 |
| 992.2 | DMMPO | Heat cramps | 0 | 0 | 0 | 1 |
| 992.3 | DMMPO | Heat exhaustion anhydrotic | 0 | 0 | 0 | 3 |
| 994.0 | DMMPO | Effects of lightning | 0 | 0 | 1 | 6 |
| 994.1 | DMMPO | Drowning and nonfatal submersion | 0 | 0 | 3 | 30 |
| 994.2 | DMMPO | Effects of deprivation of food | 0 | 0 | 0 | 30 |
| 994.3 | DMMPO | Effects of thirst | 0 | 0 | 0 | 1 |
| 994.4 | DMMPO | Exhaustion due to exposure | 0 | 0 | 0 | 7 |
| 994.5 | DMMPO | Exhaustion due to excessive | 0 | 0 | 0 | 7 |
| | | exertion | | | | |
| 994.6 | DMMPO | Motion sickness | 0 | 0 | 0 | 1 |
| 994.8 | DMMPO | Electrocution and nonfatal | 0 | 0 | 1 | 9 |
| | | effects of electric current | | | | |
| 995.0 | DMMPO | Other anaphylactic shock | 0 | 0 | 1 | 9 |
| | | not elsewhere classified | | | | |
| E991.2 | DMMPO | Injury due to war ops from | 1 | 180 | 0 | 180 |
| | | other bullets (not rubber/ | | | | |
| | | pellets) | | | | |
| E991.3 | DMMPO | Injury due to war ops from | 1 | 180 | 0 | 180 |
| | | antipersonnel bomb fragment | | | | |
| E991.9 | DMMPO | Injury due to war ops other | 1 | 180 | 0 | 180 |
| | | unspecified fragments | | | | |
| E993 | DMMPO | Injury due to war ops by other | 1 | 180 | 0 | 180 |
| | | explosion | | | | |
| V01.5 | DMMPO | Contact with or exposure to rabies | 0 | 0 | 0 | 14 |
| V79.0 | DMMPO | Screening for depression | 0 | 0 | 0 | 1 |
| 001.9 | Extended | Cholera unspecified | 0 | 0 | 2 | 5 |
| 002.0 | Extended | Typhoid fever | 0 | 0 | 0 | 5 |
| 004.9 | Extended | Shigellosis unspecified | 0 | 0 | 2 | 5 |
| 055.9 | Extended | Measles | 0 | 0 | 3 | 180 |
| 072.8 | Extended | Mumps with unspecified | 0 | 0 | 2 | 7 |
| | | complication | | | | |
| 072.9 | Extended | Mumps without complication | 0 | 0 | 0 | 7 |
| 110.9 | Extended | Dermatophytosis, of unspecified | 0 | 0 | 0 | 1 |
| | | site | | | | |
| 128.9 | Extended | Other and unspecified | 0 | 0 | 0 | 7 |
| | | Helminthiasis | | | | |
| 132.9 | Extended | Pediculosis and Phthirus | 0 | 0 | 0 | 1 |
| | | Infestation | | | | |
| 133.0 | Extended | Scabies | 0 | 0 | 0 | 1 |
| 184.9 | Extended | Malignant neoplasm of other | 0 | 0 | 0 | 180 |
| | | and unspecified female genital | | | | |
| | | organs | | | | |
| 239.0 | Extended | Neoplasms of Unspecified Nature | 1 | 7 | 0 | 5 |
| 246.9 | Extended | Unspecified Disorder of Thyroid | 0 | 0 | 0 | 5 |
| 250.00 | Extended | Diabetes Mellitus w/o | 0 | 0 | 0 | 180 |
| | | complication | | | | |
| 264.0 | Extended | Vitamin A deficiency | 0 | 0 | 0 | 3 |
| 269.8 | Extended | Other nutritional deficiencies | 0 | 0 | 0 | 3 |
| 276.51 | Extended | Volume Depletion, Dehydration | 0 | 0 | 1 | 3 |
| 277.89 | Extended | Other and unspecified disorders | 0 | 0 | 0 | 3 |
| | | of metabolism | | | | |
| 280.8 | Extended | Iron deficiency anemias | 0 | 0 | 0 | 3 |
| 300.00 | Extended | Anxiety states | 0 | 0 | 0 | 5 |
| 349.9 | Extended | Unspecified disorders of nervous | 0 | 0 | 0 | 5 |
| | | system | | | | |
| 366.00 | Extended | Cataract | 0 | 0 | 0 | 180 |
| 369.9 | Extended | Blindness and low vision | 0 | 0 | 0 | 180 |
| 372.30 | Extended | Conjunctivitis, unspecified | 0 | 0 | 0 | 2 |
| 379.90 | Extended | Other disorders of eye | 0 | 0 | 0 | 2 |
| 380.9 | Extended | Unspecified disorder of | 0 | 0 | 0 | 3 |
| | | external ear | | | | |
| 383.1 | Extended | Chronic mastoiditis | 0 | 0 | 0 | 5 |
| 386.10 | Extended | Other and unspecified | 0 | 0 | 0 | 5 |
| | | peripheral vertigo | | | | |
| 386.2 | Extended | Vertigo of central origin | 0 | 0 | 0 | 5 |
| 388.8 | Extended | Other disorders of ear | 3 | 7 | 1 | 7 |
| 411.81 | Extended | Acute coronary occlusion | 0 | 0 | 3 | 180 |
| | | without myocardial infarction | | | | |
| 428.40 | Extended | Heart failure | 0 | 0 | 3 | 180 |
| 437.9 | Extended | Cerebrovascular disease, | 0 | 0 | 3 | 180 |
| | | unspecified | | | | |
| 443.89 | Extended | Other peripheral vascular | 0 | 0 | 3 | 180 |
| | | disease | | | | |
| 459.9 | Extended | Unspecified circulatory | 0 | 0 | 3 | 180 |
| | | system disorder | | | | |
| 477.9 | Extended | Allergic rhinitis | 0 | 0 | 0 | 1 |
| 519.8 | Extended | Other diseases of respiratory | 3 | 7 | 3 | 7 |
| | | system | | | | |
| 521.00 | Extended | Dental caries | 0 | 0 | 0 | 1 |
| 522.0 | Extended | Pulpitis | 0 | 0 | 0 | 1 |
| 525.19 | Extended | Other diseases and conditions | 0 | 0 | 0 | 1 |
| | | of the teeth and supporting | | | | |
| | | structures | | | | |
| 527.8 | Extended | Diseases of the salivary | 0 | 7 | 0 | 7 |
| | | glands | | | | |
| 569.83 | Extended | Perforation of intestine | 3 | 7 | 3 | 7 |
| 571.40 | Extended | Chronic hepatitis | 0 | 0 | 0 | 180 |
| 571.5 | Extended | Cirrhosis of liver without | 0 | 0 | 3 | 180 |
| | | alcohol | | | | |
| 594.9 | Extended | Calculus of lower urinary | 3 | 3 | 1 | 5 |
| | | tract, unspecified | | | | |
| 599.8 | Extended | Urinary tract infection, | 0 | 0 | 0 | 2 |
| | | site not specified | | | | |
| 600.90 | Extended | Hyperplasia of prostate | 0 | 0 | 0 | 5 |
| 608.89 | Extended | Other disorders of male | 3 | 7 | 3 | 7 |
| | | genital organs | | | | |
| 614.9 | Extended | Inflammatory disease of | 3 | 7 | 2 | 10 |
| | | female pelvic organs/tissues | | | | |
| 616.10 | Extended | Vaginitis and vulvovaginitis | 0 | 0 | 0 | 3 |
| 623.5 | Extended | Leukorrhea not specified as | 0 | 0 | 0 | 3 |
| | | infective | | | | |
| 626.8 | Extended | Disorders of menstruation | 3 | 7 | 0 | 7 |
| | | and other abnormal bleeding | | | | |
| | | from female genital tract | | | | |
| 629.9 | Extended | Other disorders of | 0 | 0 | 0 | 3 |
| | | female genital organs | | | | |
| 650 | Extended | Normal delivery | 0 | 0 | 0 | 3 |
| 653.81 | Extended | Disproportion in pregnancy | 0 | 0 | 1 | 5 |
| | | labor and delivery | | | | |
| 690.8 | Extended | Erythematosquamous dermatosis | 0 | 0 | 0 | 1 |
| 691.8 | Extended | Atopic dermatitis and related | 0 | 0 | 0 | 1 |
| | | conditions | | | | |
| 692.9 | Extended | Contact Dermatitis, unspecified | 0 | 0 | 0 | 1 |
| | | cause | | | | |
| 693.8 | Extended | Dermatitis due to substances | 0 | 0 | 0 | 1 |
| | | taken internally | | | | |
| 696.1 | Extended | Other psoriasis and similar | 0 | 0 | 0 | 1 |
| | | disorders | | | | |
| 709.9 | Extended | Other disorders of skin and | 0 | 7 | 0 | 7 |
| | | subcutaneous tissue | | | | |
| 714.0 | Extended | Rheumatoid arthritis | 0 | 0 | 0 | 2 |
| 733.90 | Extended | Disorder of bone and cartilage, | 3 | 10 | 0 | 10 |
| | | unspecified | | | | |
| 779.9 | Extended | Other and ill-defined conditions | 0 | 0 | 1 | 2 |
| | | originating in the perinatal | | | | |
| | | period | | | | |
| 780.79 | Extended | Other malaise and fatigue | 0 | 0 | 0 | 5 |
| 780.96 | Extended | Generalized pain | 0 | 0 | 0 | 5 |
| 786.2 | Extended | Cough | 0 | 0 | 0 | 3 |
| 842.00 | Extended | Sprain of unspecified site of | 0 | 0 | 0 | 3 |
| | | wrist | | | | |

TABLE 91
EMRE Common Data: RTD Data

| PC | Type | Description | P(Adm) |
|---|---|---|---|
| 005 | DMMPO | Food poisoning bacterial | 0.0013 |
| 006 | DMMPO | Amebiasis | 0.1500 |
| 007.9 | DMMPO | Unspecified protozoal intestinal | 0.0075 |
| | | disease | |
| 008.45 | DMMPO | Intestinal infection due to | 0.0500 |
| | | clostridium difficile | |
| 008.8 | DMMPO | Intestinal infection due to other | 0.0075 |
| | | organism not classified | |
| 010 | DMMPO | Primary tb | 1.0000 |
| 037 | DMMPO | Tetanus | 1.0000 |
| 038.9 | DMMPO | Unspecified septicemia | 1.0000 |
| 042 | DMMPO | Human immunodeficiency virus | 1.0000 |
| | | [HIV] disease | |
| 047.9 | DMMPO | Viral meningitis | 0.0600 |
| 052 | DMMPO | Varicella | 1.0000 |
| 053 | DMMPO | Herpes zoster | 1.0000 |
| 054.1 | DMMPO | Genital herpes | 0.0000 |
| 057.0 | DMMPO | Fifth disease | 0.0000 |
| 060 | DMMPO | Yellow fever | 1.0000 |
| 061 | DMMPO | Dengue | 1.0000 |
| 062 | DMMPO | Mosq. borne encephalitis | 1.0000 |
| 063.9 | DMMPO | Tick borne encephalitis | 1.0000 |
| 065 | DMMPO | Arthropod-borne hemorrhagic fever | 1.0000 |
| 066.40 | DMMPO | West rale fever, unspecified | 1.0000 |
| 070.1 | DMMPO | Viral hepatitis | 0.0600 |
| 071 | DMMPO | Rabies | 1.0000 |
| 076 | DMMPO | Trachoma | 0.0009 |
| 078.0 | DMMPO | Molluscom contagiosum | 0.0000 |
| 078.1 | DMMPO | Viral warts | 0.0000 |
| 078.4 | DMMPO | Hand, foot and mouth disease | 0.0000 |
| 079.3 | DMMPO | Rhinovirus infection in conditions | 0.0050 |
| | | elsewhere and of unspecified site | |
| 079.99 | DMMPO | Unspecified viral infection | 0.0015 |
| 082 | DMMPO | Tick-borne rickettsiosis | 1.0000 |
| 084 | DMMPO | Malaria | 1.0000 |
| 085 | DMMPO | Leishmaniasis, visceral | 1.0000 |
| 086 | DMMPO | Trypanosomiasis | 1.0000 |
| 091 | DMMPO | Early primary syphilis | 0.0085 |
| 091.9 | DMMPO | Secondary syphilis, unspec | 0.0002 |
| 094 | DMMPO | Neurosyphilis | 0.0200 |
| 098.5 | DMMPO | Gonococcal arthritis | 1.0000 |
| 099.4 | DMMPO | Nongonnococcal urethritis | 0.0000 |
| 100 | DMMPO | Leptospirosis | 0.9000 |
| 274 | DMMPO | Gout | 0.0020 |
| 276 | DMMPO | Disorder of fluid, electrolyte + | 0.0000 |
| | | acid base balance | |
| 296.0 | DMMPO | Bipolar disorder, single manic | 0.4000 |
| | | episode | |
| 298.9 | DMMPO | Unspecified psychosis | 0.4000 |
| 309.0 | DMMPO | Adjustment disorder with depressed | 0.0600 |
| | | mood | |
| 309.81 | DMMPO | Ptsd | 0.4000 |
| 309.9 | DMMPO | Unspecified adjustment reaction | 0.0960 |
| 310.2 | DMMPO | Post concussion syndrome | 0.2625 |
| 345.2 | DMMPO | Epilepsy petit mal | 1.0000 |
| 345.3 | DMMPO | Epilepsy grand mal | 1.0000 |
| 346 | DMMPO | Migraine | 0.0035 |
| 361 | DMMPO | Retinal detachment | 1.0000 |
| 364.3 | DMMPO | Uveitis nos | 0.0005 |
| 365 | DMMPO | Glaucoma | 0.5000 |
| 370.0 | DMMPO | Corneal ulcer | 0.0064 |
| 379.31 | DMMPO | Aphakia | 0.0800 |
| 380.1 | DMMPO | Infective otitis externa | 0.0000 |
| 380.4 | DMMPO | Impacted cerumen | 0.0125 |
| 381 | DMMPO | Acute nonsuppurative otitis media | 0.0005 |
| 381.9 | DMMPO | Unspecified eustachian tube disorder | 0.0005 |
| 384.2 | DMMPO | Perforated tympanic membrane | 0.0008 |
| 388.3 | DMMPO | Tinnitus, unspecified | 0.0005 |
| 389.9 | DMMPO | Unspecified hearing loss | 0.4000 |
| 401 | DMMPO | Essential hypertension | 0.0006 |
| 410 | DMMPO | Myocardial infarction | 1.0000 |
| 413.9 | DMMPO | Other and unspecified angina pectoris | 1.0000 |
| 427.9 | DMMPO | Cardiac dysryhthmia unspecified | 1.0000 |
| 453.4 | DMMPO | Venous embolism/thrombus of deep | 1.0000 |
| | | vessels lower extremity | |
| 462 | DMMPO | Acute pharyngitis | 0.0011 |
| 465 | DMMPO | Acute uri of multiple or unspecified | 0.0002 |
| | | sites | |
| 466 | DMMPO | Acute bronchitis & bronchiolitis | 0.0003 |
| 475 | DMMPO | Peritonsillar abscess | 0.3375 |
| 486 | DMMPO | Pneumonia, organism unspecified | 0.0055 |
| 491 | DMMPO | Chronic bronchitis | 0.0080 |
| 492 | DMMPO | Emphysema | 0.0800 |
| 493.9 | DMMPO | Asthma | 0.0025 |
| 523 | DMMPO | Gingival and periodontal disease | 0.0000 |
| 530.2 | DMMPO | Ulcer of esophagus | 0.0006 |
| 530.81 | DMMPO | Gastroesophageal reflux | 0.0008 |
| 531 | DMMPO | Gastric ulcer | 0.0048 |
| 532 | DMMPO | Duodenal ulcer | 0.0048 |
| 540.9 | DMMPO | Acute appendicitis without mention | 1.0000 |
| | | of peritonitis | |
| 541 | DMMPO | Appendicitis, unspecified | 1.0000 |
| 550.9 | DMMPO | Unilateral inguinal hernia | 0.2633 |
| 553.1 | DMMPO | Umbilical hernia | 0.1688 |
| 553.9 | DMMPO | Hernia nos | 0.1800 |
| 564.0 | DMMPO | Constipation | 0.0000 |
| 564.1 | DMMPO | Irritable bowel disease | 0.0028 |
| 566 | DMMPO | Abscess of anal and rectal regions | 0.4500 |
| 567.9 | DMMPO | Unspecified peritonitis | 0.4500 |
| 574 | DMMPO | Cholelithiasis | 0.1875 |
| 577.0 | DMMPO | Acute pancreatitis | 0.7500 |
| 577.1 | DMMPO | Chronic pancreatitis | 0.7500 |
| 578.9 | DMMPO | Hemorrhage of gastrointestinal | 0.4050 |
| | | tract unspecified | |
| 584.9 | DMMPO | Acute renal failure unspecified | 0.2200 |
| 592 | DMMPO | Calculus of kidney | 0.0616 |
| 599.0 | DMMPO | Unspecified urinary tract infection | 0.0000 |
| 599.7 | DMMPO | Hematuria | 0.0275 |
| 608.2 | DMMPO | Torsion of testes | 0.2100 |
| 608.4 | DMMPO | Other inflammatory disorders of | 0.0788 |
| | | male genital organs | |
| 611.7 | DMMPO | Breast lump | 0.2100 |
| 633 | DMMPO | Ectopic preg | 1.0000 |
| 634 | DMMPO | Spontaneous abortion | 1.0000 |
| 681 | DMMPO | Cellulitis and abscess of finger | 0.0108 |
| | | and toe | |
| 682.0 | DMMPO | Cellulitis and abscess of face | 0.0108 |
| 682.6 | DMMPO | Cellulitis and abscess of leg | 0.0108 |
| | | except foot | |
| 682.7 | DMMPO | Cellulitis and abscess of foot | 0.0153 |
| | | except toes | |
| 682.9 | DMMPO | Cellulitis and abscess of | 0.0153 |
| | | unspecified parts | |
| 719.41 | DMMPO | Pain in joint shoulder | 0.0008 |
| 719.46 | DMMPO | Pain in joint lower leg | 0.0008 |
| 719.47 | DMMPO | Pain in joint ankle/foot | 0.0008 |
| 722.1 | DMMPO | Displacement lumbar intervertebral | 0.0135 |
| | | disc w/o myelopathy | |
| 723.0 | DMMPO | Spinal stenosis in cervical region | 0.0135 |
| 724.02 | DMMPO | Spinal stenosis of lumbar region | 0.0135 |
| 724.2 | DMMPO | Lumbago | 0.0023 |
| 724.3 | DMMPO | Sciatica | 0.0135 |
| 724.4 | DMMPO | Lumbar sprain (thoracic/lumbosacral) | 0.0149 |
| | | neuritis or radiculitis, unspec | |
| 724.5 | DMMPO | Backache unspecified | 0.0023 |
| 726.10 | DMMPO | Disorders of bursae and tendons | 0.0008 |
| | | in shoulder unspecified | |
| 726.12 | DMMPO | Bicipital tenosynovitis | 0.0008 |
| 726.3 | DMMPO | Enthesopathy of elbow region | 0.0008 |
| 726.4 | DMMPO | Enthesopathy of wrist and carpus | 0.0008 |
| 726.5 | DMMPO | Enthesopathy of hip region | 0.0008 |
| 726.6 | DMMPO | Enthesopathy of knee | 0.0008 |
| 726.7 | DMMPO | Enthesopathy of ankle and tarsus | 0.0008 |
| 729.0 | DMMPO | Rheumatism unspecified and fibrositis | 0.0008 |
| 729.5 | DMMPO | Pain in limb | 0.0008 |
| 780.0 | DMMPO | Alterations of consciousness | 0.0113 |
| 780.2 | DMMPO | Syncope | 0.0090 |
| 780.39 | DMMPO | Other convulsions | 0.0113 |
| 780.5 | DMMPO | Sleep disturbances | 0.0050 |
| 780.6 | DMMPO | Fever | 0.0010 |
| 782.1 | DMMPO | Rash and other nonspecific skin | 0.0050 |
| | | eruptions | |
| 782.3 | DMMPO | Edema | 0.0375 |
| 783.0 | DMMPO | Anorexia | 0.0050 |
| 784.0 | DMMPO | Headache | 0.0113 |
| 784.7 | DMMPO | Epistaxis | 0.0050 |
| 784.8 | DMMPO | Hemorrhage from throat | 0.0113 |
| 786.5 | DMMPO | Chest pain | 0.0113 |
| 787.0 | DMMPO | Nausea and vomiting | 0.0050 |
| 787.91 | DMMPO | Diarrhea nos | 0.0013 |
| 789.00 | DMMPO | Abdominal pain unspecified site | 0.0113 |
| 800.0 | DMMPO | Closed fracture of vault of skull | 1.0000 |
| | | without intracranial injury | |
| 801.0 | DMMPO | Closed fracture of base of skull | 1.0000 |
| | | without intracranial injury | |
| 801.76 | DMMPO | Open fracture base of skull with | 1.0000 |
| | | subarachnoid, subdural and | |
| | | extradural hemorrhage with loss | |
| | | of consciousness of unspecified | |
| | | duration | |
| 802.0 | DMMPO | Closed fracture of nasal bones | 1.0000 |
| 802.1 | DMMPO | Open fracture of nasal bones | 1.0000 |
| 802.6 | DMMPO | Fracture orbital floor closed | 1.0000 |
| | | (blowout) | |
| 802.7 | DMMPO | Fracture orbital floor open | 1.0000 |
| | | (blowout) | |
| 802.8 | DMMPO | Closed fracture of other facial | 1.0000 |
| | | bones | |
| 802.9 | DMMPO | Open fracture of other facial | 1.0000 |
| | | bones | |
| 805 | DMMPO | Closed fracture of cervical vertebra | 1.0000 |
| | | w/o spinal cord injury | |
| 806.1 | DMMPO | Open fracture of cervical vertebra | 1.0000 |
| | | with spinal cord injury | |
| 806.2 | DMMPO | Closed fracture of dorsal vertebra | 1.0000 |
| | | with spinal cord injury | |
| 806.3 | DMMPO | Open fracture of dorsal vertebra with | 1.0000 |
| | | spinal cord injury | |
| 806.4 | DMMPO | Closed fracture of lumbar spine with | 1.0000 |
| | | spinal cord injury | |
| 806.5 | DMMPO | Open fracture of lumbar spine with | 1.0000 |
| | | spinal cord injury | |
| 806.60 | DMMPO | Closed fracture sacrum and coccyx | 1.0000 |
| | | w/unspec. spinal cord injury | |
| 806.70 | DMMPO | Open fracture sacrum and coccyx | 1.0000 |
| | | w/unspec. spinal cord injury | |
| 807.0 | DMMPO | Closed fracture of rib(s) | 1.0000 |
| 807.1 | DMMPO | Open fracture of rib(s) | 1.0000 |
| 807.2 | DMMPO | Closed fracture of sternum | 1.0000 |
| 807.3 | DMMPO | Open fracture of sternum | 1.0000 |
| 808.8 | DMMPO | Fracture of pelvis unspecified, closed | 1.0000 |
| 808.9 | DMMPO | Fracture of pelvis unspecified, open | 1.0000 |
| 810.0 | DMMPO | Clavicle fracture, closed | 1.0000 |
| 810.1 | DMMPO | Clavicle fracture, open | 1.0000 |
| 810.12 | DMMPO | Open fracture of shaft of clavicle | 1.0000 |
| 811.0 | DMMPO | Fracture of scapula, closed | 1.0000 |
| 811.1 | DMMPO | Fracture of scapula, open | 1.0000 |
| 812.00 | DMMPO | Fracture of unspecified part of | 1.0000 |
| | | upper end of humerus, closed | |
| 813.8 | DMMPO | Fracture unspecified part of radius | 1.0000 |
| | | and ulna closed | |
| 813.9 | DMMPO | Fracture unspecified part of radius | 1.0000 |
| | | and ulna open | |
| 815.0 | DMMPO | Closed fracture of metacarpal bones | 1.0000 |
| 816.0 | DMMPO | Phalanges fracture, closed | 1.0000 |
| 816.1 | DMMPO | Phalanges fracture, open | 1.0000 |
| 817.0 | DMMPO | Multiple closed fractures of hand | 1.0000 |
| | | bones | |
| 817.1 | DMMPO | Multiple open fracture of hand bones | 1.0000 |
| 820.8 | DMMPO | Fracture of femur neck, closed | 1.0000 |
| 820.9 | DMMPO | Fracture of femur neck, open | 1.0000 |
| 821.01 | DMMPO | Fracture shaft femur, dosed | 1.0000 |
| 821.11 | DMMPO | Fracture shaft of femur, open | 1.0000 |
| 822.0 | DMMPO | Closed fracture of patella | 1.0000 |
| 822.1 | DMMPO | Open fracture of patella | 1.0000 |
| 823.82 | DMMPO | Fracture tib fib, closed | 1.0000 |
| 823.9 | DMMPO | Fracture of unspecified part of | 1.0000 |
| | | tibia and fibula open | |
| 824.8 | DMMPO | Fracture ankle, nos, closed | 1.0000 |
| 824.9 | DMMPO | Ankle fracture, open | 1.0000 |
| 825.0 | DMMPO | Fracture to calcaneus, closed | 1.0000 |
| 826.0 | DMMPO | Closed fracture of one or more | 1.0000 |
| | | phalanges of foot | |
| 829.0 | DMMPO | Fracture of unspecified bone, | 1.0000 |
| | | closed | |
| 830.0 | DMMPO | Closed dislocation of jaw | 1.0000 |
| 830.1 | DMMPO | Open dislocation of jaw | 1.0000 |
| 831 | DMMPO | Dislocation shoulder | 0.6750 |
| 831.04 | DMMPO | Closed dislocation of | 1.0000 |
| | | acromioclavicular joint | |
| 831.1 | DMMPO | Dislocation of shoulder, open | 1.0000 |
| 832.0 | DMMPO | Dislocation elbow, closed | 1.0000 |
| 832.1 | DMMPO | Dislocation elbow, open | 1.0000 |
| 833 | DMMPO | Dislocation wrist closed | 1.0000 |
| 833.1 | DMMPO | Dislocated wrist, open | 1.0000 |
| 834.0 | DMMPO | Dislocation of finger, closed | 0.0000 |
| 834.1 | DMMPO | Dislocation of finger, open | 1.0000 |
| 835 | DMMPO | Closed dislocation of hip | 1.0000 |
| 835.1 | DMMPO | Hip dislocation open | 1.0000 |
| 836.0 | DMMPO | Medial meniscus tear | 0.0750 |
| 836.1 | DMMPO | Lateral meniscus tear | 0.0750 |
| 836.2 | DMMPO | Meniscus tear of knee | 0.0750 |
| 836.5 | DMMPO | Dislocation knee, closed | 1.0000 |
| 836.6 | DMMPO | Other dislocation of knee open | 1.0000 |
| 839.01 | DMMPO | Closed dislocation first cervical | 1.0000 |
| | | vertebra | |
| 840.4 | DMMPO | Rotator cuff sprain | 0.0375 |
| 840.9 | DMMPO | Sprain shoulder | 0.0375 |
| 843 | DMMPO | Sprains and strains of hip and thigh | 0.0375 |
| 844.9 | DMMPO | Sprain, knee | 0.0250 |
| 845 | DMMPO | Sprain of ankle | 0.0125 |
| 846 | DMMPO | Sprains and strains of socroiliac | 0.3750 |
| | | region | |
| 846.0 | DMMPO | Sprain of lumbosacral (joint) | 0.3750 |
| | | (ligament) | |
| 847.2 | DMMPO | Sprain lumbar region | 0.0375 |
| 847.3 | DMMPO | Sprain of sacrum | 0.0375 |
| 848.1 | DMMPO | Jaw sprain | 0.0375 |
| 848.3 | DMMPO | Sprain of ribs | 0.0375 |
| 850.9 | DMMPO | Concussion | 0.8000 |
| 851.0 | DMMPO | Cortex (Cerebral) contusion w/o | 1.0000 |
| | | open intracranial wound | |
| 851.01 | DMMPO | Cortex (Cerebral) contusion w/o | 1.0000 |
| | | open wound no loss of consciousness | |
| 852 | DMMPO | Subarachnoid subdural extradural | 1.0000 |
| | | hemorrhage injury | |
| 853 | DMMPO | Other and unspecified intracranial | 1.0000 |
| | | hemorrhage injury w/o open wound | |
| 853.15 | DMMPO | Unspecified intracranial hemorrhage | 1.0000 |
| | | with open intracranial wound | |
| 860.0 | DMMPO | Traumatic pneumothorax w/o open wound | 1.0000 |
| | | into thorax | |
| 860.1 | DMMPO | Traumatic pneumothorax w/open wound | 1.0000 |
| | | into thorax | |
| 860.2 | DMMPO | Traumatic hemothorax w/o open wound | 1.0000 |
| | | into thorax | |
| 860.3 | DMMPO | Traumatic hemothorax with open wound | 1.0000 |
| | | into thorax | |
| 860.4 | DMMPO | Traumatic pneumohemothorax w/o open | 1.0000 |
| | | wound thorax | |
| 860.5 | DMMPO | Traumatic pneumohemothorax with open | 1.0000 |
| | | wound thorax | |
| 861.0 | DMMPO | Injury to heart w/o open wound | 1.0000 |
| | | into thorax | |
| 861.10 | DMMPO | Unspec. injury of heart w/open | 1.0000 |
| | | wound into thorax | |
| 861.2 | DMMPO | Injury to lung, nos, closed | 1.0000 |
| 861.3 | DMMPO | Injury to lung nos, open | 1.0000 |
| 863.0 | DMMPO | Stomach injury, w/o open wound | 1.0000 |
| | | into cavity | |
| 864.10 | DMMPO | Unspecified injury to liver with | 1.0000 |
| | | open wound into cavity | |
| 865 | DMMPO | Injury to spleen | 1.0000 |
| 866.0 | DMMPO | Injury kidney w/o open wound | 1.0000 |
| 866.1 | DMMPO | Injury to kidney with open wound | 1.0000 |
| | | into cavity | |
| 867.0 | DMMPO | Injury to bladder urethra without | 1.0000 |
| | | open wound into cavity | |
| 867.1 | DMMPO | Injury to bladder and urethrea with | 1.0000 |
| | | open wound into cavity | |
| 867.2 | DMMPO | Injury to ureter w/o open wound | 1.0000 |
| | | into cavity | |
| 867.3 | DMMPO | Injury to ureter with open wound | 1.0000 |
| | | into cavity | |
| 867.4 | DMMPO | Injury to uterus w/o open wound | 1.0000 |
| | | into cavity | |
| 867.5 | DMMPO | Injury to uterus with open wound | 1.0000 |
| | | into cavity | |
| 870 | DMMPO | Open wound of ocular adnexa | 0.9405 |
| 870.3 | DMMPO | Penetrating wound of orbit without | 0.9405 |
| | | foreign body | |
| 870.4 | DMMPO | Penetrating wound of orbit with | 0.9405 |
| | | foreign body | |
| 871.5 | DMMPO | Penetration of eyeball with | 1.0000 |
| | | magnetic foreign body | |
| 872 | DMMPO | Open wound of ear | 0.0250 |
| 873.4 | DMMPO | Open wound of face without mention | 0.3000 |
| | | of complication | |
| 873.8 | DMMPO | Open head wound w/o complication | 0.6840 |
| 873.9 | DMMPO | Open head wound with complications | 1.0000 |
| 874.8 | DMMPO | Open wound of other and unspecified | 0.6840 |
| | | parts of neck w/o complications | |
| 875.0 | DMMPO | Open wound of chest (wall) without | 0.3000 |
| | | complication | |
| 876.0 | DMMPO | Open wound of back without | 0.8000 |
| | | complication | |
| 877.0 | DMMPO | Open wound of buttock without | 0.0100 |
| | | complication | |
| 878 | DMMPO | Open wound of genital organs | 1.0000 |
| | | (external) including traumatic | |
| | | amputation | |
| 879.2 | DMMPO | Open wound of abdominal wail | 0.3000 |
| | | anterior w/o complication | |
| 879.6 | DMMPO | Open wound of other unspecified | 0.8000 |
| | | parts of trunk without | |
| | | complication | |
| 879.8 | DMMPO | Open wound(s) (multiple) of | 0.8000 |
| | | unspecified site(s) w/o | |
| | | complication | |
| 880 | DMMPO | Open wound of the shoulder and | 0.0400 |
| | | upper arm | |
| 881 | DMMPO | Open wound elbows, forearm, and | 0.0040 |
| | | wrist | |
| 882 | DMMPO | Open wound hand except fingers | 1.0000 |
| | | alone | |
| 883.0 | DMMPO | Open wound of fingers without | 0.8000 |
| | | complication | |
| 884.0 | DMMPO | Multiple/unspecified open wound | 1.0000 |
| | | upper limb without complication | |
| 885 | DMMPO | Traumatic amputation of thumb | 0.8000 |
| | | (complete) (partial) | |
| 886 | DMMPO | Traumatic amputation of other | 1.0000 |
| | | finger(s) (complete) (partial) | |
| 887 | DMMPO | Traumatic amputation of arm and | 1.0000 |
| | | hand (complete) (partial) | |
| 890 | DMMPO | Open wound of hip and thigh | 0.7200 |
| 891 | DMMPO | Open wound of knee leg (except | 0.7200 |
| | | thigh) and ankle | |
| 892.0 | DMMPO | Open wound foot except toes alone | 0.8000 |
| | | w/o complication | |
| 894.0 | DMMPO | Multiple/unspecified open wound of | 0.4480 |
| | | lower limb w/o complication | |
| 895 | DMMPO | Traumatic amputation of toe(s) | 1.0000 |
| | | (complete) (partial) | |
| 896 | DMMPO | Traumatic amputation of foot | 1.0000 |
| | | (complete) (partial) | |
| 897 | DMMPO | Traumatic amputation of leg(s) | 1.0000 |
| | | (complete) (partial) | |
| 903 | DMMPO | Injury to blood vessels of upper | 1.0000 |
| | | extremity | |
| 904 | DMMPO | Injury to blood vessels of lower | 1.0000 |
| | | extremity and unspec. sites | |
| 910.0 | DMMPO | Abrasion/friction burn of face, | 0.0000 |
| | | neck, scalp w/o infection | |
| 916.0 | DMMPO | Abrasion/friction burn of hip, | 0.0000 |
| | | thigh, leg, ankle w/o infection | |
| 916.1 | DMMPO | Abrasion/friction burn of hip, | 0.9000 |
| | | thigh, leg, ankle with infection | |
| 916.2 | DMMPO | Blister hip & leg | 0.0000 |
| 916.3 | DMMPO | Blister of hip thigh leg and ankle | 0.9000 |
| | | infected | |
| 916.4 | DMMPO | Insect bite nonvenom hip, thigh, | 0.0000 |
| | | leg, ankle w/o infection | |
| 916.5 | DMMPO | Insect bite nonvenom hip, thigh, | 0.9000 |
| | | leg, ankle, with infection | |
| 918.1 | DMMPO | Superficial injury cornea | 0.0000 |
| 920 | DMMPO | Contusion of face scalp and neck | 0.0000 |
| | | except eye(s) | |
| 921.0 | DMMPO | Black eye | 0.0000 |
| 922.1 | DMMPO | Contusion of chest wall | 0.0000 |
| 922.2 | DMMPO | Contusion of abdominal wall | 0.0000 |
| 922.4 | DMMPO | Contusion of genital organs | 0.0010 |
| 924.1 | DMMPO | Contusion of knee and lower leg | 0.0000 |
| 924.2 | DMMPO | Contusion of ankle and foot | 0.0000 |
| 924.3 | DMMPO | Contusion of toe | 0.0000 |
| 925 | DMMPO | Crushing injury of face, scalp & | 1.0000 |
| | | neck | |
| 926 | DMMPO | Crushing injury of trunk | 1.0000 |
| 927 | DMMPO | crushing injury of upper limb | 1.0000 |
| 928 | DMMPO | Crushing injury of lower limb | 1.0000 |
| 930 | DMMPO | Foreign Body on External Eye | 0.0000 |
| 935 | DMMPO | Foreign body in mouth, esophagus | 1.0000 |
| | | and stomach | |
| 941 | DMMPO | Burn of face, head, neck | 0.0000 |
| 942.0 | DMMPO | Burn of trunk, unspecified degree | 1.0000 |
| 943.0 | DMMPO | Burn of upper limb except wrist | 1.0000 |
| | | and hand unspec. degree | |
| 944 | DMMPO | Burn of wrist and hand | 1.0000 |
| 945 | DMMPO | Burn of tower limb(s) | 1.0000 |
| 950 | DMMPO | Injury to optic nerve and pathways | 1.0000 |
| 953.0 | DMMPO | Injury to cervical nerve root | 1.0000 |
| 953.4 | DMMPO | Injury to brachial plexus | 1.0000 |
| 955.0 | DMMPO | Injury to axillary nerve | 1.0000 |
| 956.0 | DMMPO | Injury to sciatic nerve | 1.0000 |
| 959.01 | DMMPO | Other and unspecified injury to | 0.7600 |
| | | head | |
| 959.09 | DMMPO | Other and unspecified injury to | 0.7600 |
| | | face and neck | |
| 959.7 | DMMPO | Other and unspecified injury to | 0.7600 |
| | | knee leg ankle and foot | |
| 989.5 | DMMPO | Toxic effect of venom | 0.0050 |
| 989.9 | DMMPO | Toxic effect unspec subst chiefly | 1.0000 |
| | | nonmedicinal/source | |
| 991.3 | DMMPO | Frostbite | 1.0000 |
| 991.6 | DMMPO | Hypothermia | 1.0000 |
| 992.0 | DMMPO | Heat stroke and sun stroke | 1.0000 |
| 992.2 | DMMPO | Heat cramps | 0.0000 |
| 992.3 | DMMPO | Heat exhaustion anhydrotic | 0.0000 |
| 994.0 | DMMPO | Effects of lightning | 0.3800 |
| 994.1 | DMMPO | Drowning and nonfatal submersion | 1.0000 |
| 994.2 | DMMPO | Effects of deprivation of food | 1.0000 |
| 994.3 | DMMPO | Effects of thirst | 0.0000 |
| 994.4 | DMMPO | Exhaustion due to exposure | 0.3800 |
| 994.5 | DMMPO | Exhaustion due to excessive exertion | 0.3800 |
| 994.6 | DMMPO | Motion sickness | 0.0000 |
| 994.8 | DMMPO | Electrocution and nonfatal effects | 1.0000 |
| | | of electric current | |
| 995.0 | DMMPO | Other anaphylactic shock not | 1.0000 |
| | | elsewhere classified | |
| E991.2 | DMMPO | Injury due to war ops from other | 1.0000 |
| | | bullets (not rubber/pellets) | |
| E991.3 | DMMPO | Injury due to war ops from anti- | 1.0000 |
| | | personnel bomb fragment | |
| E991.9 | DMMPO | Injury due to war ops other | 1.0000 |
| | | unspecified fragments | |
| E993 | DMMPO | Injury due to war ops by other | 1.0000 |
| | | explosion | |
| V01.5 | DMMPO | Contact with or exposure to rabies | 1.0000 |
| V79.0 | DMMPO | Screening for depression | 0.0000 |
| 001.9 | Extended | Cholera unspecified | 1.0000 |
| 002.0 | Extended | Typhoid fever | 1.0000 |
| 004.9 | Extended | Shigellosis unspecified | 1.0000 |
| 055.9 | Extended | Measles | 1.0000 |
| 072.8 | Extended | Mumps with unspecified complication | 1.0000 |
| 072.9 | Extended | Mumps without complication | 1.0000 |
| 110.9 | Extended | Dermatophytosis, of unspecified site | 0.0000 |
| 128.9 | Extended | Other and unspecified Helminthiasis | 0.0013 |
| 132.9 | Extended | Pediculosis and Phthirus Infestation | 0.0000 |
| 133.0 | Extended | Scabies | 0.0000 |
| 184.9 | Extended | Malignant neoplasm of other and | 1.0000 |
| | | unspecified female genital organs | |
| 239.0 | Extended | Neoplasms of Unspecified Nature | 0.1400 |
| 246.9 | Extended | Unspecified Disorder of Thyroid | 1.0000 |
| 250.00 | Extended | Diabetes Mellitus w/o complication | 0.3500 |
| 264.0 | Extended | Vitamin A deficiency | 0.0000 |
| 269.8 | Extended | Other nutritional deficiencies | 0.0375 |
| 276.51 | Extended | Volume Depletion, Dehydration | 0.0000 |
| 277.89 | Extended | Other and unspecified disorders | 0.0400 |
| | | of metabolism | |
| 280.8 | Extended | Iron deficiency anemias | 1.0000 |
| 300.00 | Extended | Anxiety states | 0.1500 |
| 349.9 | Extended | Unspecified disorders of nervous | 1.0000 |
| | | system | |
| 366.00 | Extended | Cataract | 1.0000 |
| 369.9 | Extended | Blindness and low vision | 1.0000 |
| 372.30 | Extended | Conjunctivitis, unspecified | 0.0000 |
| 379.90 | Extended | Other disorders of eye | 0.0684 |
| 380.9 | Extended | Unspecified disorder of external | 0.0038 |
| | | ear | |
| 383.1 | Extended | Chronic mastoiditis | 1.0000 |
| 386.10 | Extended | Other and unspecified peripheral | 0.9000 |
| | | vertigo | |
| 386.2 | Extended | Vertigo of central origin | 1.0000 |
| 388.8 | Extended | Other disorders of ear | 0.0180 |
| 411.81 | Extended | Acute coronary occlusion without | 1.0000 |
| | | myocardial infarction | |
| 428.40 | Extended | Heart failure | 1.0000 |
| 437.9 | Extended | Cerebrovascular, disease, unspecified | 1.0000 |
| 443.89 | Extended | Other peripheral vascular disease | 0.8550 |
| 459.9 | Extended | Unspecified circulatory system disorder | 0.8550 |
| 477.9 | Extended | Allergic rhinitis | 0.0000 |
| 519.8 | Extended | Other diseases of respiratory system | 0.9000 |
| 521.00 | Extended | Dental caries | 1.0000 |
| 522.0 | Extended | Pulpitis | 1.0000 |
| 525.19 | Extended | Other diseases and conditions of the | 1.0000 |
| | | teeth and supporting structures | |
| 527.8 | Extended | Diseases of the salivary glands | 0.3375 |
| 569.83 | Extended | Perforation of intestine | 1.0000 |
| 571.40 | Extended | Chronic hepatitis | 1.0000 |
| 571.5 | Extended | Cirrhosis of liver without alcohol | 1.0000 |
| 594.9 | Extended | Calculus of lower urinary tract, | 1.0000 |
| | | unspecified | |
| 599.8 | Extended | Urinary tract infection, site not | 0.2200 |
| | | specified | |
| 600.90 | Extended | Hyperplasia of prostate | 1.0000 |
| 608.89 | Extended | Other disorders of male genital organs | 0.2100 |
| 614.9 | Extended | Inflammatory disease of female pelvic | 0.2040 |
| | | organs/tissues | |
| 616.10 | Extended | Vaginitis and vulvovaginitis | 0.0000 |
| 623.5 | Extended | Leukorrhea not specified as infective | 0.7125 |
| 626.8 | Extended | Disorders of menstruation and other | 0.7125 |
| | | abnormal bleeding from female | |
| | | genital tract | |
| 629.9 | Extended | Other disorders of female genital | 0.1496 |
| | | organs | |
| 650 | Extended | Normal delivery | 1.0000 |
| 653.81 | Extended | Disproportion in pregnancy labor and | 1.0000 |
| | | delivery | |
| 690.8 | Extended | Erythematosquamous dermatosis | 0.0090 |
| 691.8 | Extended | Atopic dermatitis and related conditions | 0.0015 |
| 692.9 | Extended | Contact Dermatitis, unspecified cause | 0.0001 |
| 693.8 | Extended | Dermatitis due to substances taken | 0.0140 |
| | | internally | |
| 696.1 | Extended | Other psoriasis and similar disorders | 0.4500 |
| 709.9 | Extended | Other disorders of skin and subcutaneous | 0.0135 |
| | | tissue | |
| 714.0 | Extended | Rheumatoid arthritis | 1.0000 |
| 733.90 | Extended | Disorder of bone and cartilage, | 0.0900 |
| | | unspecified | |
| 779.9 | Extended | Other and ill-defined conditions | 1.0000 |
| | | originating in the perinatal | |
| | | period | |
| 780.79 | Extended | Other malaise and fatigue | 0.9310 |
| 780.96 | Extended | Generalized pain | 0.7600 |
| 786.2 | Extended | Cough | 0.0760 |
| 842.00 | Extended | Sprain of unspecified site of wrist | 0.0750 |

What is claimed is:

1) A method for assessing medical risks of a planned mission comprising:
a) establishing a patient condition occurrence frequencies (PCOF) scenario for a planned mission;
b) stimulating the planned mission to create a set of mission-centric PCOF distributions;
c) presenting the mission-centric PCOF distributions to a user;
d) ranking patient conditions based on their mission-centric PCOF distribution;
e) identifying medical risks of said planned mission.

2) The method of claim 1, wherein said step a) further comprising
a) obtaining information about a plurality of missions, each said mission has a predefined PCOF scenario;
b) selecting a predefined PCOF scenario for the planned mission, wherein said PCOF scenario is represented by a plurality sets of baseline PCOF distributions (discrete probability distribution that provides the probability of a particular illness or injury);
c) presenting PCOF adjustment factors applicable to said selected mission;
d) modifying said set of baseline PCOF distributions of said selected PCOF scenario manually or using one or more of said PCOF adjustment factors to create a set of customized PCOF scenario for said planned mission.

3) The method of claim 2, wherein the set of baseline PCOF distributions is modified at a patient type category level, a ICD-9 category level or a ICD-9 subcategory level, whereas the sum of the proportions of all applicable patient type categories, the ICD-9 categories or the ICD-9 subcategories for said customized PCOF scenario is equal to 1, respectively.

4) The method of claim 2, wherein the PCOF adjustment factors comprises Age, Gender, OB/GYN Correction, Geographic Region, Response Phase, Season or Country.

5) The method of claim 4, wherein one or more PCOF adjustment factors that can be applied to a selected set of baseline PCOF distributions is restricted according to table 1 based on the patient type and the selected PCOF scenario.

6) The method of claim 4, wherein said PCOF adjustment factors are calculated based at least partially on user inputs.

7) The method of claim 1, wherein said planned mission comprising ground operation, shipboard operation, fixed-base combat operation, humanitarian assistance (HA) operation, or disaster relief (DR) operation.

8) The method of claim 1, wherein medical risks of said planned mission is patient conditions with the highest distribution.

9) A method for assessing adequacy of a medical support plan for a mission, comprising
a) establishing a mission scenario for a planned mission;
b) stimulating the planned mission to:
i. create a set of mission-centric PCOF;
ii. generate estimated casualties for the planned mission; and
iii. calculate estimated medical requirements for the planned mission; and
c) Assess adequacy of the medical support plan using mission-centric PCOF distributions, estimated casualties and estimated medical requirements.

10) A method of estimating medical requirement of a planned mission,
a) establishing a mission scenario for a planned mission;
b) creating a set of mission-centric PCOF;
c) generating estimated casualties for the planned mission;
d) calculating estimated medical requirements for the planned mission, and
e) setting up medical logistic operation for said planned mission using said estimated medical requirement.

11) The method of claim 10, wherein the medical requirements comprising:
a) the number of hours of operating room time needed;
b) the number of operating room tables needed;
c) the number of intensive care unit beds needed;
d) the number of ward beds needed;
e) the total number of ward and ICU beds needed;
f) the number of staging beds needed;
g) the number of patients evacuated after being treated in the ward;
h) the total number of patients evacuated from the ward and ICU;
i) the number of red blood cell units needed;
j) the number of fresh frozen plasma units needed;
k) the number of platelet concentrate units needed; and
l) the number of Cryoprecipitate units needed.
